(19) United States
(12) Patent Application Publication
(10) Pub. No.: US 2017/0000837 A1
(43) Pub. Date: Jan. 5, 2017

(54) Prognostic and Diagnostic Kits and Herbal Therapies for Treating Skin Conditions, Autoimmune Diseases, Inflammatory Ailments and Cancer

(71) Applicant: Sirbal Ltd., Limassol (CY)

(72) Inventors: Nadav Shraibom, Herzelia (IL); Anu T. Singh, Noida (IN); Manu Jaggi, DLF City (IN); Eran Steinberg, San Francisco, CA (US); Ritu Verma, Ghaziabad (IN); Alka Madaan, Ghaziabad (IN)

(21) Appl. No.: 15/197,745

(22) Filed: Jun. 29, 2016

Related U.S. Application Data
(63) Continuation-in-part of application No. 15/131,743, filed on Apr. 18, 2016.
Application No. 15/131,743 is a continuation-in-part of application No. 14/754,266, filed on Jun. 29, 2015.
Application No. 14/754,266 is a continuation-in-part of application No. 13/900,525, filed on May 22, 2013, now Pat. No. 9,095,606.
(60) Provisional application No. 62/198,637, filed on Jul. 29, 2015.
(60) Provisional application No. 62/297,796, filed on Feb. 19, 2016.
(60) Provisional application No. 62/313,709, filed on Mar. 26, 2016.
(60) Provisional application No. 62/325,993, filed on Apr. 21, 2016.
(60) Provisional application No. 62/348,762, filed on Jun. 10, 2016.
(60) Provisional application No. 62/355,614, filed on Jun. 28, 2016.
(60) Provisional application No. 62/259,056, filed on Nov. 23, 2015.
(60) Provisional application No. 62/268,226, filed on Dec. 16, 2015.

(51) Int. Cl. A61K 36/708; A61K 36/355; A61K 8/97; G01N 33/68; A61K 9/00; A61K 45/06; G01N 33/573; G01N 33/574; A61K 36/64; A61Q 5/02

(52) U.S. Cl. CPC A61K 36/708; A61K 36/64; A61K 36/355; A61K 8/97; A61Q 5/02; A61K 9/0014; A61K 45/06; G01N 33/573; G01N 33/574; G01N 33/68; G01N 2800/60

(57) ABSTRACT

A treatment regimen for psoriasis, eczema or other skin ailment, inflammation, autoimmune disease, leukemia, melanoma or other cancer includes administering to a patient a known or discovered treatment regimen along with periodic doses of an herbal combination including Sheng Di Huang, Da Huang or Jin Yin Hua or combinations thereof.

### PRIORITY AND RELATED APPLICATIONS

This application is a continuation-in-part (CIP) of U.S. patent application Ser. No. (USSN) 15/131,743, filed Apr. 18, 2016; which claims priority to U.S. Ser. No. 62/198,637, filed Jul. 29, 2015; and U.S. Ser. No. 62/297,796, filed Feb. 19, 2016; and which is a CIP of U.S. Ser. No. 14/754,266 filed Jun. 29, 2015; which is a CIP of (USSN) U.S. Ser. No. 13/900,525, filed May 22, 2013, now U.S. Pat. No. 9,095,606; which is incorporated by reference.

This application also claims priority to U.S. Ser. No. 62/313,709, filed Mar. 26, 2016; and to U.S. Ser. No. 62/325,993, filed Apr. 21, 2016; and to U.S. Ser. No. 62/348,762, filed Jun. 10, 2016; which are incorporated by reference.

This application also claims priority to U.S. Ser. No. 62/355,614, filed Jun. 28, 2016; U.S. Ser. No. 62/259,056, filed Nov. 23, 2015; and to U.S. Ser. No. 62/268,226, filed Dec. 16, 2015; which are incorporated by reference.

This application is related to one of two contemporaneously-filed applications by the same Applicant and Inventors that are entitled: Prognostic and Diagnostic Methods and Herbal Therapies for Treating Skin Conditions, Autoimmune Diseases, Inflammatory Ailments and Cancer; and Prognostic and Diagnostic Kits and Herbal Therapies for Treating Skin Conditions, Autoimmune Diseases, Inflammatory Ailments and Cancer; each filed on Jun. 29, 2016; which are incorporated by reference.

This application is also related to U.S. patent applications Nos. 62/268,226; 62/259,056; 62/348,762; 15/133,056; 14/754,266; PCT/U515/38341; U.S. Ser. Nos. 14/710,865; 14/815,892; 14/287,158; 14/287,153; 13/890,990; PCT/1B13/02975; U.S. Ser. Nos. 14/981,899; 14/815,705; 13/152,039; PCT/IB11/03078; and 61/413,430; and US published patent applications nos. 20160051553A1; 20160136220A1; 20160136219A1; 20160136216A1; 20160136223A1; 20160136222A1; 20160136221A1; 20160113983A1; 20160143980A1; 20160136218A1; 20140205685A1; and 20140206631A1; and U.S. Pat. Nos. 9,066,974; 9,095,606; 8,734,859; 8,597,695; and 8,541,382; all of which are incorporated by reference.

### BACKGROUND OF THE INVENTION

1. Field of the Invention

The present invention relates generally to disease treatments, including treatments for psoriasis, eczema, melanoma and other skin disorders, inflammatory and autoimmune ailments and cancer. More specifically it concerns the use of combinations of certain herbs, certain herbal extracts and/or certain herbal molecular components alone or in combination with known antimetabolite, antifolate, anti-inflammatory, or autoimmune treatments and other known and/or described treatments, particularly to treat psoriasis or reduce psoriatic suffering and symptoms, or to treat eczema or blisters, redness or eczematic soreness or itching or crusty skin caused by eczema, or to treat inflammation or an inflammatory condition, or to treat melanoma or other cancer, or to reduce white cell count, tumor size or painfulness from cancer, or to treat or reduce suffering from another skin ailment, or to treat an autoimmune disease or disorder, or otherwise to treat or to reduce suffering from one or more of diseases causing or stemming from inflammation or autoimmune disease or to administer along with a known or discovered treatment to enhance effectiveness, reduce toxicity or side effects and/or to facilitate weening from a known or discovered treatment. Examples include herbal formulas including one or more of, or a combination of two or more of, Da Huang, Sheng Di Huang, and Jin Yin Hua, and/or combinations including one or more of Mu Dan Pi, Di Gu Pi, Xian He Cao, and/or Chun Gen Pi., and/or another herb, molecule or extract, or combination of herbs, molecules or extracts described herein.

2. Description of the Related Art

Herbal medicines are prevalent, and serve the medicinal needs of a large population around the world. The global herbal medicine market is currently worth around $30 billion. There is also an increased effort for the isolation of bioactive phytochemicals from herbs for their possible usefulness in the control of various ailments.

It is desired to provide more reliable medical kits that include diagnostic test kits and medicines for treating a patient in accordance with an indication provided by the test kit.

It is desired to provide more reliable prognostic kits that include medicines and prognostic measuring devices and prognostic indicators. With such prognostic kits, more effective stratification of patients is provided for certain treatments for certain conditions such that patients can be treated more safely and effectively.

It is therefore desired to provide diagnostic test kits configured to measure levels of certain panels of one or more diagnostic markers that are most indicative of a patient's disposition with a disease, condition or disorder, or combination or ailments.

It is also desired to provide prognostic test kits configured to measure levels of certain panels of one or more prognostic markers that are most indicative of a patient's tendency to respond safely and effectively to certain treatments or combinations of treatments for certain diseases, conditions or disorders.

It is also desired to have an herbal and/or molecular combination that may be administered to a patient as a safe and effective treatment of a condition.

It is also desired to have an herbal and/or molecular combination that may be administered to a patient before, during and/or after a typical, known or discovered treatment regimen to enhance the effectiveness of such known or discovered treatments, and/or to reduce side effects of such known or discovered treatments and/or for weening a patient from a dependence on such known or discovered treatments.

### BRIEF DESCRIPTION OF THE DRAWINGS

FIGS. 1-3 show plots of growth of cancer cells versus dilution factor for each of 18 different herbs.

FIG. 4 is a bar chart that illustrates the effects of certain herbs and combinations of herbs on live cancer cells.

FIGS. 5A-5B schematically illustrate example steps in a cooking process in accordance with certain embodiments.

FIG. 6 schematically illustrates example steps on a tablet formulation process in accordance with certain embodiments.

FIG. 7 illustrates the components of a shampoo in accordance with certain embodiments.

FIG. 8 illustrates a hair growth cycle and a hair fall control strategy in accordance with certain embodiments.

FIG. 9 illustrates certain targets of combinations of Da Huang, Sheng Di Huang and Jin Yin Hua in accordance with certain embodiments.

FIG. 10 illustrates certain putative mechanisms of combinations of Da Huang Sheng Di Huang and Jin Yin Hua in accordance with certain embodiments.

FIG. 11 illustrates certain psoriasis target therapies for administration along with combinations of Da Huang Sheng Di Huang and Jin Yin Hua in accordance with certain embodiments.

FIG. 12 illustrates certain rheumatoid arthritis (RA) target therapies for administration along with combinations of Da Huang Sheng Di Huang and Jin Yin Hua in accordance with certain embodiments.

FIG. 13 illustrates certain chronic lymphocytic leukemia (CLL) target therapies for administration along with combinations of Da Huang Sheng Di Huang and Jin Yin Hua in accordance with certain embodiments.

FIG. 14 illustrates certain Alzheimer's neuro-inflammation target therapies for administration along with combinations of Da Huang Sheng Di Huang and Jin Yin Hua in accordance with certain embodiments.

### DETAILED DESCRIPTIONS OF THE EMBODIMENTS

A method is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, proctitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The method includes measuring an elevated or low level, or higher or lower than expected level, or higher or lower than a normal or threshold level, of each of one or more of, or combinations of levels of two or three or multiple or several or many or a panel of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a first bodily fluid sample or skin sample, or both, extracted from a patient. The method further includes formulating a diagnosis of scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, for the patient based on said measuring said elevated and/or low level or combination of levels. The method further includes administering a medicinal composition that comprises an effective dose between 20-160 mg/kg of an herbal combination of Da Huang, Sheng Di Huang and Jin Yin Hua to the patient.

Another method is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (MS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The method includes measuring an elevated and/or low level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a first bodily fluid or skin sample, or both, extracted from a patient. The method further includes formulating a diagnosis of scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, for the patient based on said measuring said elevated and/or low level or combination of levels. The method also includes administering a medicinal composition that comprises an effective dose between 1.0 wt. %-15 wt. % of an herbal combination of Da Huang, Sheng Di Huang and Jin Yin Hua to the patient.

A treatment method may also include measuring a reduced level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a second bodily fluid or skin sample, or both, extracted from the patient after administering said medicinal composition; and repeating the administering of said medicinal composition to said patient based on said measuring said reduced level or combination of levels.

A medicinal composition may include between 1.0 wt. %-15 wt. % of the herbal combination of Da Huang, Sheng Di Huang and Jin Yin Hua. The medicinal composition comprises 2.5 wt. % or more of the herbal combination of Da Huang, Sheng Di Huang and Jin Yin Hua. The medicinal composition may include 10 wt. % or less of the herbal combination of Da Huang, Sheng di Huang and Jin Yin Hua. The medicinal composition may include 2.5 wt. %-5.0 wt. % of the herbal combination of Da Huang, Sheng Di Huang and Jin Yin Hua.

A medicinal composition may include an effective dose between 1-6 grams of said herbal combination for a 50 kg patient or 2-12 grams for a 100 kg patient. The effective dose may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

A medicinal composition may be formulated as a shampoo and applied to the scalp. The medicinal composition may be formulated as a shampoo, conditioner, cream, lotion, ointment or other topical scalp or hair treatment. The medicinal composition may be formulated as a cream, lotion, ointment or other topical skin treatment.

A method may further include administering, in combination with said medicinal composition that comprises effective doses between 20-160 mg/kg of an herbal combination of Da Huang, Sheng Di Huang and Jin Yin Hua, one or more additional medicines comprising Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof.

A method may further include administering, in combination with said medicinal composition that comprises effective doses between 20-160 mg/kg of an herbal combination of Da Huang, Sheng Di Huang and Jin Yin Hua, one or more additional medicines comprising an inhibitor or enabler of a biologic, protein, molecule or receptor component of a patient, such as IL-12, IL-23, TNF alpha, JAK, STAT, IL-17, PDE4, p40 subunit of IL-12 and/or IL-23 and/or a humanized p40 monoclonal antibody, IL-22, IL-20, IL-23/p19, JAK3, TH0, Th1, Th17 and/or Th22 cell, IFN alpha or IFN gamma, IL-15, IL-17R, IL-18, IL-19, IL-21, sPLA2, NO (nitric oxide), VEGF, IL-24, kinase, tyrosine, topoisomerase, IL-1, IL-2, IL-6, IL-8, BTK, SYK, ZAP-70, PI3KCD, AKT, ALK, TRPV1, TRPA1, MRGPRA3, PAR2, mucunain or other proteases, chloroquine, pruritogen, cowhage, sapsaicin, HER-2, FLT-3, MAPK1, BRAF, MEK1, PD-1, CD279, PD-L1, LYN, ABL, FLT3, KIT, LCK, JAK1, PLC, BCR, P13K delta, TGF betal, GMCSF, MCP-1, AKT1 (PKB alpha), ERBB2 (HER2), FLT3, MAPK1 (ERK2), PRKCA (PKC alpha), BRAF, BRAF V599E, MAP2K1 (MEK1), PGE2-PG4, PTGER4, PTGS2, or COX2, or combinations thereof. A medicine, medical kit, diagnostic kit, prognostic kit and method of preparing a medicine are each also accordingly provided.

A method may further include administering, in combination with said medicinal composition that comprises effective doses between 20-160 mg/kg of an herbal combination of Da Huang, Sheng Di Huang and Jin Yin Hua, one or more additional medicines comprising a dermal or epidermal inflammation, neutrophil, monocyte chemotaxis, antimicrobial defense mechanism, pruritus, acanthosis, parakeratosis, hyperkeratosis, angiogenesis.

A medicine is also provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, proctitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof.

The medicine includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose including between 20-160 mg/kg of an herbal combination of Da Huang, Sheng Di Huang and Jin Yin Hua.

Effective doses of the medicine may include between 1-6 grams of said herbal combination for a 50 kg patient or 2-12 grams for a 100 kg patient.

Effective doses of the medicine may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses of the medicine may include 1.0 wt. %-15.0 wt. % of said herbal combination of Da Huang, Sheng Di Huang and Jin Yin Hua.

Effective doses of the medicine may include 2.5 wt. %-5.0 wt. % of said herbal combination of Da Huang, Sheng Di Huang and Jin Yin Hua.

Another medicine is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, proctitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicine includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose comprising between 1.0wt. %-15.0wt. % of an herbal combination of Da Huang, Sheng Di Huang and Jin Yin Hua.

Effective doses of the medicine may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses of the medicine may include 1-6 grams of an herbal combination of Da Huang, Sheng Di Huang and Jin Yin Hua for a 50 kg patient or 2-12 grams for a 100 kg patient. Effective doses of the medicine may include 2.5 wt. %-5.0 wt. % of said herbal combination of Da Huang, Sheng Di Huang and Jin Yin Hua.

A medical kit is provided that includes a diagnostic kit and a medicinal formulation. The diagnostic kit includes a test kit and an indicator. The test kit is configured for measuring a level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a bodily fluid or skin sample, or both, and said indicator for providing a diagnostic result based on said measured level or combinations of levels and on one or more expected correlations between said level or combination of levels and manifestation of one or more diseases including scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicinal formulation includes a shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof. The medicinal formulation includes a predetermined number of one or more effective doses including between 20-160 mg/kg of an herbal combination of Da Huang, Sheng Di Huang and Jin Yin Hua.

The medical kit may further include a prognostic kit that includes a prognostic test kit and a prognostic indicator. The prognostic test kit may be configured for measuring a level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a second bodily fluid or skin sample, or both, after a prognostic period of treatment. The prognostic indicator may be configured for providing a prognostic result for the patient based on a comparison of said measured level or combination of levels in the first and second bodily fluid or skin sample, or both.

Another medical kit is provided that includes a diagnostic kit and a medicinal formulation. The diagnostic kit includes a test kit and an indicator. The test kit is configured for measuring a level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a bodily fluid or skin sample, or both, and said indicator for providing a diagnostic result based on said measured level or combinations of levels and on one or more expected correlations between said level or combination of levels and manifestation of one or more diseases including scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicinal formulation includes a shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof. The medicinal formulation includes a predetermined number of one or more effective doses between 1.0 wt. %-15.0 wt. % of an herbal combination of Da Huang, Sheng di Huang and Jin Yin Hua.

The medical kit may also include a prognostic kit including a prognostic test kit and a prognostic indicator. The prognostic test kit may be configured for measuring a level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in a second bodily fluid or skin sample, or both, after a prognostic period of treatment. The prognostic indicator may be configured for providing a prognostic result for the patient based on a comparison of said measured level or combination of levels in the first and second bodily fluid or skin sample, or both.

Effective doses of the medicinal formulation may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses of the medicinal formulation may include 1-6 grams of said herbal combination of Da Huang, Sheng Di Huang and Jin Yin Hua for a 50 kg patient or 2-12 grams for a 100 kg patient.

Effective doses of the medicinal formulation may include 2.5 wt. %-5.0 wt. % of said herbal combination of Da Huang, Sheng Di Huang and Jin Yin Hua.

Effective doses of the medicinal formulation may include between 20-160 mg/kg of an herbal combination of Da Huang, Sheng Di Huang and Jin Yin Hua.

Effective doses of the medicinal formulation may include between 20-160 mg/kg of an herbal combination of Da Huang, Sheng Di Huang and Jin Yin Hua.

A prognostic kit is also provided that includes a medicinal formulation, and a prognostic test kit and indicator. The medicinal formulation includes shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof. The medicinal formulation includes a predetermined number of one or more effective doses between 20-160 mg/kg of an herbal combination of Da Huang, Sheng Di Huang and Jin Yin Hua. The prognostic test kit is configured for measuring a level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in first and second bodily fluid or skin samples, or both, respectively extracted from a patient before and after a prognostic period of treatment with the medicinal formulation. The prognostic indicator is configured for providing a prognostic result for the patient based on a comparison of the measured level or combination of levels in the first and second bodily fluid or skin samples, or both.

Effective doses of the medicinal formulation may include 1.0 wt. %-15.0 wt. % of an herbal combination of Da Huang, Sheng Di Huang and Jin Yin Hua.

Effective doses of the medicinal formulation may include 2.5wt. %-5.0 wt. % of the herbal combination of Da Huang, Sheng Di Huang and Jin Yin Hua.

Another prognostic kit is provided. The prognostic kit includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses each including between 1.0 wt. %-15.0 wt. % of an herbal combination of Da Huang, Sheng Di Huang and Jin Yin Hua. The prognostic kit includes a prognostic test kit configured for measuring a level or combination of levels of one or more inflammatory cytokines, growth factors; cytokine receptors; ligand to surface markers, chemokines, protoangiogenic mediators, automicrobial proteins, neuropeptides, oxidative stress markers, dendritic cells (DC), plasmacytoid DC, monocytes, macrophages, histamines, T&B lymphocytes, or theranostic biomarkers, or combinations thereof, in first and second bodily fluid or skin samples, or both, respectively extracted from a patient before and after a prognostic period of treatment with said medicinal formulation, and a prognostic indicator configured for providing a prognostic result for the patient based on a comparison of measured levels or combinations of levels from said first and second bodily fluid or skin samples, or both.

Effective doses of an herbal combination of Da Huang, Sheng Di Huang and Jin Yin Hua may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses of an herbal combination of Da Huang, Sheng Di Huang and Jin Yin Hua may include 1-6 grams of the herbal combination for a 50 kg patient or 2-12 grams for a 100 kg patient.

Effective doses may include 2.5 wt. %-5.0 wt. % of the herbal combination of Da Huang, Sheng Di Huang and Jin Yin Hua.

Effective doses may include between 20-160 mg/kg of an herbal combination of Da Huang, Sheng Di Huang and Jin Yin Hua.

Another method is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, proctitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The method includes measuring a level or combination of levels of one or more of IL-5, IL-13, IL-17, IL-23, TNF-α, IL-6, IL-7, IL-13, GMCSF, MCP-1, IL-1-β, IL-1-RA, IL-8, IL-9, IL-10, IL-12, IL-4, IL-19, IL-20, IL-21, IL-22, IL-24, IL-25, IL-26, IL-31, IL-33, IL-17A, IL-17F, eotaxin, FGF-β, G-CSF, IFN-γ, IP-10, sCD40L, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a first bodily fluid or skin sample, or both, extracted from a patient. The method also includes formulating a diagnosis of scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, for the patient based on said measured level or combination of levels and on one or more expected correlations between said level or combination of levels and manifestation within said patient of one or more diseases. The method also includes administering a medicinal composition that comprises an effective dose between 20-160 mg/kg of an herbal combination of Da Huang, Sheng Di Huang and Jin Yin Hua to the patient to treat the patient in accordance with said diagnosis.

The method may include measuring a level or combination of levels of one or more of IL-5, IL-13, IL-17, IL-23, TNF-α, IL-6, IL-7, IL-13, IL-15, GM-CSF, MCP-1, MCP-1(MCAF), IL-1-β, IL-1-RA, IL-8, IL-9, IL-10, IL-12, IL-12(p70), IL-19, IL-20, IL-22, IL-24, IL-26, IL-1ra, IL-2, eotaxin, FGF basic, FGF-β, G-CSF, IFN-γ, IP-10, MIP-1, PDGFRB, MIP-1-α, MIP-1-γ, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, (32M, NO, LTB4, PLA2, or PDE4, OR A KINASE, E.G., AKT1(PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid or skin sample, or both, extracted from the patient after administering said medicinal composition over a prognostic period; and indicating to repeat the administering of the medicinal composition that comprises an effective dose between 20-160 mg/kg of an herbal combination of Da Huang, Sheng Di Huang and Jin Yin Hua to said patient a significant number of further times based on a comparison between said measured levels or combinations of levels within said first and second bodily fluid or skin samples, or both, and on an expected correlation between certain differences between measured levels or combinations of levels in bodily fluid samples respectively extracted before and after a prognostic period of administration of said medicinal composition.

The medicinal composition may include between 1.0 wt. %-15 wt. % of said herbal combination of Da Huang, Sheng Di Huang and Jin Yin Hua. The medicinal composition may include 2.5 wt. % or more of said herbal combination of Da Huang, Sheng Di Huang and Jin Yin Hua. The medicinal composition may include lOwt.% or less of said herbal combination of Da Huang, Sheng di Huang and Jin Yin Hua.

The medicinal composition may include 2.5 wt. %-5.0 wt. % of said herbal combination of Da Huang, Sheng Di Huang and Jin Yin Hua.

The medicinal composition may include an effective dose between 1-6 grams of said herbal combination for a 50 kg patient or 2-12 grams for a 100 kg patient. Effective doses may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

The medicinal composition may be formulated as a shampoo and applied to the scalp.

The medicinal composition may be formulated as a shampoo, conditioner, cream, lotion, ointment or other topical scalp or hair treatment.

The medicinal composition may be formulated as a cream, lotion, ointment or other topical skin treatment.

The may further include administering, in combination with said medicinal composition that comprises effective doses between 20-160 mg/kg of an herbal combination of Da Huang, Sheng Di Huang and Jin Yin Hua, one or more additional medicines including Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof.

The method may further include administering, in combination with said medicinal composition that comprises effective doses between 20-160 mg/kg of an herbal combination of Da Huang, Sheng Di Huang and Jin Yin Hua, one or more additional medicines including an IL-12, IL-23, TNF alpha, JAK, STAT, IL-17, PDE4, p40 subunit of IL-12 and/or IL-23 and/or a humanized p40 monoclonal antibody, IL-22, IL-20, IL-23/p19, JAK3, Th1, Th17 and/or Th22 cell, IFN gamma, IL-17R, IL-19, sPLA2, NO (nitric oxide), VEGF, IL-24, kinase, tyrosine, topoisomerase, IL-1, IL-6, IL-8, BTK, SYK, ZAP-70, PI3KCD, AKT, HER-2, FLT-3, MAPK1, BRAF, MEK1, PD-1, CD279, PD-L1, LYN, ABL, FLT3, KIT, LCK, JAK1, PLC, BCR, P13K delta, GMCSF, MCP-1, AKT1 (PKB alpha), ERBB2 (HER2), FLT3, MAPK1 (ERK2), PRKCA (PKC alpha), BRAF, BRAF V599E or MAP2K1 (MEK1) inhibitor, or combinations thereof.

Another method is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The method includes measuring a level or combination of levels of one or more of IL-5, IL-13, IL-17, IL-23, TNF-α, IL-6, IL-7, IL-13, GMCSF, MCP-1, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGF-RB, PDGF-BB, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, f32M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a first bodily fluid, or skin sample, or both, or serum or sera, extracted from a patient. The method also includes formulating a diagnosis of scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, for the patient based on said measured level or combination of levels and on one or more expected correlations between said level or combination of levels and manifestation within said patient of one or more diseases. The method also includes administering a medicinal composition that comprises an effective dose between 1.0 wt. %-15 wt. % of an herbal combination of Da Huang, Sheng Di Huang and Jin Yin Hua for a 50 kg patient or 2-12 grams for a 100 kg patient to treat the patient in accordance with said diagnosis.

The method may also include measuring a level or combination of levels of one or more of IL-5, IL-13, IL-17, IL-23, TNF-α, IL-6, IL-7, IL-13, GMCSF, MCP-1, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid or skin sample, or both, extracted from the patient after administering said medicinal composition over a prognostic period; and indicating to repeat the administering of effective doses of said medicinal composition to said patient a significant number of further times based on a comparison between said measured levels or combinations of levels within said first and second bodily fluid or skin samples, or both, and on an expected correlation between certain differences between said measured levels or combinations of levels in bodily fluid samples respectively extracted before and after the prognostic period of administration of said medicinal composition.

The medicinal composition may include between 1-6 grams of said herbal combination of Da Huang, Sheng Di Huang and Jin Yin Hua for a 50 kg patient or 2-12 grams for a 100 kg patient.

The medicinal composition may include 2.5 wt. % or more of said herbal combination of Da Huang, Sheng Di Huang and Jin Yin Hua. The medicinal composition may include 10 wt. % or less of said herbal combination of Da Huang, Sheng di Huang and Jin Yin Hua.

The medicinal composition may include 2.5 wt. %-5.0 wt. % of said herbal combination of Da Huang, Sheng Di Huang and Jin Yin Hua.

Effective doses may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

The medicinal composition may be formulated as a shampoo and applied to the scalp.

The medicinal composition may be formulated as a shampoo, conditioner, cream, lotion, ointment or other topical scalp or hair treatment.

The medicinal composition may be formulated as a cream, lotion, ointment or other topical skin treatment.

The method may further include administering, in combination with said medicinal composition that comprises effective doses between 1.0 wt. %-15 wt. % of an herbal combination of Da Huang, Sheng Di Huang and Jin Yin Hua, one or more additional medicines including Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof.

The method may further include administering, in combination with said medicinal composition that comprises effective doses between 1.0 wt. %-15 wt. % of an herbal combination of Da Huang, Sheng Di Huang and Jin Yin Hua, one or more additional medicines including an IL-12, IL-23, TNF alpha, JAK, STAT, IL-17, PDE4, p40 subunit of IL-12 and/or IL-23 and/or a humanized p40 monoclonal antibody, IL-22, IL-20, IL-23/p19, JAK3, Th1, Th17 and/or Th22 cell, IFN gamma, IL-17R, IL-19, sPLA2, NO (nitric oxide), VEGF, IL-24, kinase, tyrosine, topoisomerase, IL-1, IL-6, IL-8, BTK, SYK, ZAP-70, PI3KCD, AKT, HER-2, FLT-3, MAPK1, BRAF, MEK1, PD-1, CD279, PD-L1, LYN, ABL, FLT3, KIT, LCK, JAK1, PLC, BCR, P13K delta, GMCSF, MCP-1, AKT1 (PKB alpha), ERBB2 (HER2), FLT3, MAPK1 (ERK2), PRKCA (PKC alpha), BRAF, BRAF V599E or MAP2K1 (MEK1) inhibitor, or combinations thereof.

Another medicine is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (MS), colitis, proctitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicine includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose including between 20-160 mg/kg of an herbal combination of Da Huang, Sheng Di Huang and Jin Yin Hua.

Effective doses may include between 1-6 grams of said herbal combination for a 50 kg patient or 2-12 grams for a 100 kg patient. Effective doses may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses may include 1.0 wt. %-15.0 wt. % of said herbal combination of Da Huang, Sheng Di Huang and Jin Yin Hua.

Effective doses may include 2.5 wt. %-5.0 wt. % of said herbal combination of Da Huang, Sheng Di Huang and Jin Yin Hua.

Another medicine is provided for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicine includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that includes a predetermined number of one or more effective doses, each effective dose including between 1.0 wt. %-15.0 wt. % of said herbal combination of Da Huang, Sheng Di Huang and Jin Yin Hua.

Effective doses may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses may include 1-6 grams of said herbal combination for a 50 kg patient or 2-12 grams for a 100 kg patient.

Effective doses may include 2.5 wt. %-5.0 wt. % of said herbal combination of Da Huang, Sheng Di Huang and Jin Yin Hua.

A medical kit is providing including a diagnostic kit and a medicinal formulation. The diagnostic kit includes a test kit and an indicator. The test kit is configured for measuring a level or combination of levels of one or more of IL-5, IL-13, IL-17, IL-23, TNF-α, IL-6, IL-7, IL-13, GMCSF, MCP-1, IL-9, IL-1β, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, MIP-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a bodily fluid or skin sample, or both. The indicator is configured for providing a diagnostic result based on said measured level or combinations of levels and on one or more expected correlations between said level or combination of levels and manifestation of one or more diseases including scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, proctitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicinal formulation includes shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, said medicinal formulation comprising a predetermined number of one or more effective doses, each effective dose including between 20-160 mg/kg of an herbal combination of Da Huang, Sheng Di Huang and Jin Yin Hua.

The medical kit may also include a prognostic kit including a prognostic test kit and a prognostic indicator, wherein the prognostic test kit is configured for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, βM, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid or skin sample, or both, after a prognostic period of treatment, and wherein the prognostic indicator is configured for providing a prognostic result for the patient based on a comparison of said measured level or combination of levels in the first and second bodily fluid or skin sample, or both.

Another medical kit is provided that includes a diagnostic kit and a medicinal formulation. The diagnostic kit includes a test kit and an indicator. The test kit is configured for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a bodily fluid or skin sample, or both. The indicator is configured for providing a diagnostic result based on said measured level or combinations of levels and on one or more expected correlations between said level or combination of levels and manifestation of one or more diseases including scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof. The medicinal formulation includes shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof. The medicinal formulation includes a predetermined number of one or more effective doses, each effective dose including between 1.0 wt. %-15 wt. % of an herbal combination of Da Huang, Sheng di Huang and Jin Yin Hua.

The medical kit may also include a prognostic kit that includes a prognostic test kit and a prognostic indicator. The prognostic test kit may be configured for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid or skin sample, or both, after a prognostic period of treatment. The prognostic indicator may be configured for providing a prognostic result for the patient based on a comparison of said measured level or combination of levels in the first and second bodily fluid or skin sample, or both.

Effective doses may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses may include 1-6 grams of said herbal combination for a 50 kg patient or 2-12 grams for a 100 kg patient.

Effective doses may include 2.5 wt. %-5.0 wt. % of said herbal combination of Da Huang, Sheng Di Huang and Jin Yin Hua. Effective doses may include between 20-160 mg/kg of an herbal combination of Da Huang, Sheng Di Huang and Jin Yin Hua.

Effective doses may include between 20-160 mg/kg of an herbal combination of Da Huang, Sheng Di Huang and Jin Yin Hua.

Another prognostic kit is provided that includes a medicinal formulation and a prognostic test kit and indicator. The medicinal formulation includes shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, said medicinal formulation comprising a predetermined number of one or more effective doses, each effective dose including between 20-160 mg/kg of an herbal combination of Da Huang, Sheng Di Huang and Jin Yin Hua. The prognostic test kit is configured for measuring a level or combination of levels of one or more of IL-5, IL-13, IL-17, IL-23, TNF-α, IL-6, IL-7, IL-13, GMCSF, MCP-1, IL-9, IL-113, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in first and second bodily fluid or skin samples, or both, respectively extracted from a patient before and after a prognostic period of treatment with said medicinal formulation. The prognostic indicator is configured for providing a prognostic result for the patient based on a comparison of said measured level or combination of levels in the first and second bodily fluid or skin samples, or both.

Effective doses may include 1.0 wt. %-15.0 wt. % of said herbal combination of Da Huang, Sheng Di Huang and Jin Yin Hua.

Effective doses may include 2.5 wt. %-5.0 wt. % of said herbal combination of Da Huang, Sheng Di Huang and Jin Yin Hua.

Another prognostic kit is provided that includes a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose including between 1.0 wt. %-15.0 wt. % of an herbal combination of Da Huang, Sheng Di Huang and Jin Yin Hua.

The prognostic kit also includes a prognostic test kit for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, or a kinase, e.g., AKT1 (PKB alpha), ERBB2 (HER2), FLT3, MAPK1 (ERK2), PRKCA (PKC alpha), BRAF, BRAF V599E or MAP2K1 (MEK1), or combinations thereof, in first and second bodily fluid or skin samples, or both, respectively extracted from a patient before and after a prognostic period of treatment with said medicinal formulation. The prognostic kit also includes a prognostic indicator for providing a prognostic result for the patient based on a comparison of measured levels or combinations of levels from said first and second bodily fluid or skin samples, or both.

Effective doses may include 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

Effective doses may include 1-6 grams of said herbal combination for a 50 kg patient or 2-12 grams for a 100 kg patient.

Effective doses may include 2.5 wt. %-5.0 wt. % of said herbal combination of Da Huang, Sheng Di Huang and Jin Yin Hua.

Effective doses may include between 20-160 mg/kg of an herbal combination of Da Huang, Sheng Di Huang and Jin Yin Hua.

Herbal combinations of Da Huang, Sheng Di Huang and Jin Yin Hua are provided for treatment of certain diseases. One or more described herbs, herbal extracts and/or molecules are also provided for administration to a patient before, during and/or after administering a treatment regimen with a known medicine, or in a combination therapy, to enhance the effectiveness and/or reduce expected side effects. Herbal combinations including Da Huang, Sheng Di Huang and Jin Yin Hua alone or in combination with one or more other herbs, herbal extracts, molecules and/or other medicines may be used to treat skin ailments or dermatologic conditions such as scalp psoriasis, atopic dermatitis, eczema, herpes, shingles, psoriatic, seborrheic dermatitis, acne, skin lesions, UV exposure, burns, warts, skin delayed type hypersensitivity disorder, and dandruff, as well as inflammation, chronic inflammation and inflammatory diseases such as arthritis, rheumatoid arthritis, juvenile rheumatoid arthritis, refractory rheumatoid arthritis, osteoarthritis, appendicitis, bursitis, colitis, cystitis, dermatitis, phlebitis, vasculitis, rhinitis, tendonitis, tonsillitis and arthritic psoriasis, as well as Alzheimer's disease, Parkinson's disease, multiple sclerosis, IBS, diabetes, hypertension, allergies, asthma, and adult respiratory distress syndrome, as well as autoimmune conditions such as psoriasis, colitis, pemphigus vulgaris, vitiligo, celiac disease, Hashimoto's thyroiditis, SLE, Sjögren's syndrome, sarcoidosis, scleroderma, cryoglobulinemic vasculitis, and dermatomyositis, as well as osteoporosis/bone resorption, cardiovascular disease, metabolic syndrome, hypotension, atherosclerosis, coronary artery disease, depression, and cancers such as melanoma, CLL, AML, leukemia and breast, colon and/or prostate cancer.

Markers may be used in formulating a prognosis and/or a diagnosis for a patient, particularly those demonstrating strong correlation with dispositions within patients of certain diseases such as those described above and below herein and/or those demonstrating effectiveness of administration to patients of certain treatments including herbal combinations of Da Huang, Sheng Di Huang and Jin Yin Hua. Markers may be utilized in diagnosis and treatment and/or in formulating a prognosis for treatment with a dosage composition including Da Huang, Sheng Di Huang and Jin Yin Hua alone or in combination with other medicines and/or with other herbs, extracts or molecules.

Markers may be selected from the following non-exhaustive listing of marker types: Inflammatory cytokines; Growth factors; Cytokine receptors; Ligand to surface markers; Chemokines; Protoangiogenic mediators; Automicrobial proteins; Neuropeptides; Oxidative stress markers; Dendritic cells (DC); Plasmacytoid DC; Monocytes; Macrophages, histamines; T&B lymphocytes; anti-immune modulators, adhesion molecules, osteoclasts, osteoblasts, tyrosine kinases, and/or Theranostic biomarkers.

Levels of certain markers having known correlations with dispositions of certain diseases may be measured in patient bodily samples, e.g., serum, skin, blood, saliva, for determining a diagnosis and treatment. Levels of certain markers having known correlations with effectiveness of certain treatments including herbal combinations of Da Huang, Sheng Di Huang and Jin Yin Hua, alone or in a combination therapy, may be tested both before and after administration of one or more doses of the herbal therapy or combination therapy for determining a prognosis as to whether the herbal therapy or combination therapy is likely to be effective for this patient.

Among these markers are IL-5, IL-13, IL-17, IL-23 and TNF-α which have shown strong correlation to disposition with certain inflammatory, auto-immune, oncological, and/or dermatological diseases, as well as to responsiveness to administered dosage compositions including Da Huang, Sheng Di Huang and Jin Yin Hua by downregulation in diagnosed patients following treatment. Further example markers include IL-6, IL-7, IL-13, GMCSF and MCP-1 which also showed good correlations both with these diseases and the herbal treatments.

Another example marker is IL-9 which has shown significant correlation with the diseases and herbal treatments.

Additional example inflammatory, autoimmune, dermatologic and oncologic markers include IL-1-β, IL-1-RA, IL-8, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, MMP, PI3K-δ, PIK3CD, PIK3R1 CCL3, CCL4, PLC, BCR, CD-5, CD-38, CD-19, CD-20, CD-79a, β2M, NO, LTB4, PLA2, and PDE4.

Psoriasis and eczema/dermatitis are inflammatory, autoimmune and dermatologic diseases. Current management strategies include oral medications, steroid creams and light therapy. An aqueous mixture of three herbs: Rheum palmatum L. (Da Huang), Rehmannia glutinosa Libosch (Sheng di huang) and Lonicera Japonica (Jin Yin Hua) is provided in cream, lotion or shampoo formulation, and in tablets, skin patches, subdermal injections and in nano-formulations including sub-450 nm, sub-350 nm, sub-250 nm, sub-150 nm and sub-100 nm particulate sizes, in an approximate ratio of one part (±30%) Da Huang, one part (±30%) Jin Yin Hua and two-four parts Sheng Di Huang. The aforementioned three herb formulations have demonstrated promising anti-psoriatic and anti-eczematic activity in pre-clinical and clinical studies. Further testing is ongoing for determining efficacies in fighting others of the diseases described above and below herein. Herbal formulations including a majority to an entirety of the three herbs Da Huang, Sheng Di Huang and Jin Yin Hua have demonstrated significant inhibitory effects on secretion of TNF-α, TARC and VEGF in keratinocytes (HaCaT), considerable downregulation of IL-6 in RAW264.7 cells and IgE in human myeloma cell line-U-266, as well as inhibition of JAK-1/JAK-3. In addition, lotion, cream, gel, shampoo, foam, subdermal injections, IV, and oral formulations are provided herein.

Diagnostic test kits are provided in accordance with certain embodiments that include diagnosis-based treatments, diagnostic testing that involve novel markers or novel combinations of markers or novel reagents or combinations thereof. An example of a diagnostic test kit in accordance with certain embodiments includes a test kit for measuring certain markers in patient bodily samples (e.g., serum or sera from skin washings, liquid blood serum, keritinocytes, or fibroblasts, or combinations thereof). The example test kit may further include an indicator for communicating a diagnosis to the patient based on the results of the measuring and known correlations between levels of markers and predispositions with one or more diseases, conditions, ailments or disorders. A shampoo, cream, lotion, pills, skin patches, gel, nanogel, injection pens, ointments, subdermal injections, IV bags, tablets, capsules, nano-lipid carriers, nano-chrystals, nano-particulates, subcutaneous implants, stents, or combinations thereof, may be provided with the test kit or may be advised based on the diagnosis, for administering treatment to the patient based on the diagnosis.

Prognostic test kits are also provided in accordance with certain embodiments that include a test kit for taking bodily samples from a patient (e.g., skin samples, such as serum or sera from skin washings, liquid blood serum, keritinocytes or fibroblasts, or combinations thereof), and measuring a panel of markers in the bodily samples that have been preselected based on determined/tested relevance to a disease based on a diagnosis (e.g., a kit that simultaneously analyzes multiple cytokines, and may use multiplex technology). The test kit may include a medicine including a combination of one, two or all three of Da Huang, Sheng Di Huang and Jin Yin Hua, alone or in combination with one or more further herbs, molecules or other medicine. The prognostic kit includes a second test kit for taking a second bodily sample and measuring a panel of one or more markers in the second bodily sample. The prognostic kit includes an indicator for communicating a prognosis to the patient based on whether one or more markers went up or down with administration of the medicine and on determined correlations between measured affects on levels of certain markers and administration of the medicine.

A patient stratification method may include categorizing patients based on their particular prognoses (e.g., such that administration of certain medicines such as combinations of Da Huang, Sheng Di Huang and Jin Yin Hua, would only be advised for those patients showing at least a minimum likelihood of response to medicine. A theranostic approach to treatment of the patient may be developed based on the diagnostic and/or prognostic test results.

Combinations of Da Huang, Sheng Di Huang and Jin Yin Hua may be administered along with one or more developed therapies to enhance their effectiveness and/or to reduce certain side effects of the developed therapies and/or to ease a transition off of a developed therapy upon completion of what can be for some patients a grueling, albeit effective, regimen of the developed therapy. Such developed therapies may include one or more of Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol, tazarotene, Otezla, Embrel, Humira, Cinzia, Cosentyx, Remicade, Simponi, Taltz, Steroids, Tacrolimus, Prograf, Cyclosporine, Sulfasalazine, NSAIDS such as naproxen, diclofenac, ibuprofen, and aspirin, acetaminophen, Hydrochloroquine, Corticosteroids, Leflunomide, Biologic DMARDs (disease-modifying antirheumatic drugs), Infliximab, Adalimumab, Anakinra, Dasatinib, Fostamatinib, Rituximab, Tocilizumab and Abatacept, GS-1101, Ibrutinib, steroid cream, Stelara, L. casei, Omega-3 fatty acids, antioxidants, green coffee beans, ginger, garlic, and turmeric.

Methods and medicinal compositions are provided for treating psoriasis, eczema, or another other autoimmune or inflammatory condition, or melanoma or another skin ailment, or leukemia or other cancer, or another ailment described herein. The methods include preparing an herbal composition, determining a diagnosis or prognosis for a patient, administering an herbal composition to a patient diagnosed with one or more of these ailments and/or stratifying a patient population based on prognosis. A medicinal composition may include a combination of two or more of Da Huang, Sheng Di Huang and Jin Yin Hua alone or in a majority proportion with one or more other herbal or molecular constituents.

An herbal medicinal composition may be administered alone or in combination with an established treatment regimen such as any of those described herein before, during or after administration of the established regimen. An example two herb combination in accordance with certain embodiments includes Da Huang and Sheng Di Huang. Another two herb combination includes Da Huang and Jin Yin Hua. Another two herb combination includes Sheng Di Huang and Jin Yin Hua. A three herb combination includes Da Huang, Sheng Di Huang and Jin Yin Hua.

Each of these three herbs may be used as single herb treatments that may have efficacy at or above certain minimum doses and non-toxicity at or below certain maximum doses. Several specific dosage examples are provided herein for these herbal combinations. Da Huang is provided as an example of a one herb treatment that may be combined in a treatment regimen along with an established regimen of another known treatment, such as those described above and below herein. In combination, however, these two and three herb combinations, alone or in combination with methotrexate or another known treatment described herein, form enhanced combinative treatments for psoriasis, inflammation and skin ailments such as melanoma and eczema, and for pain, and for certain cancers such as CLL and AML and for autoimmune diseases and other ailments described herein.

Other combinations of two to seven herbs selected from among the following seven herbs may be administered to a patient as an effective treatment or supplement to an established treatment regimen in accordance with certain embodiments, the seven herbs including: Da Huang, Sheng Di Huang, and Jin Yin Hua, as well as Mu Dan Pi, Di Gu Pi, Xian He Cao, and Chun Gen Pi. Herbal combinations including a majority proportion of one or more of these seven herbs may also include one or more other herbal or molecular constituents, such as any of those that are described above or below herein and/or that may be known to those skilled in the art.

Other herbal or molecular combinations or herbal supplements may be administered in combination therapies along with an established treatment regimen, such as any of those described herein, for treating certain ailments, such as one or more of those described herein, alone or in further combination with one or more of emodin or digoxin, or both, and/or one or more of aucubin, vanillic acid, carvacrol, beta-sitosterol, rhein or rhapontin or one or more additional molecules or remedies described herein. Herbal and molecular combinations may include other molecules, molecular extracts or molecular combinations that are described above or below herein and/or that may be known to those skilled in the art.

Other combinations may include an herbal combination of Da Huang, Sheng Di Huang and Jin Yin Hua with an established treatment, such as any of those described herein or understood by those skilled in the art. The medicinal composition of an established regimen with an herbal combination of Da Huang and Sheng Di Huang or with an herbal combination of Da Huang, Sheng Di Huang and Jin Yin Hua may be used as an effective, non-toxic treatment for skin ailments such as psoriasis, eczema, or melanoma, or inflammatory disease, or autoimmune disorder, or cancer. The combination of an established treatment regimen, such as any of those described herein, with an herbal combination of one or more of Da Huang, Sheng Di Huang and Jin Yin Hua can be used to provide a treatment that exhibits an increased effectiveness and/or reduced toxicity compared with treatments involving the established treatment regimen alone alone or otherwise without an herbal combination that includes a majority proportion of two or more of Da Huang, Sheng Di Huang and Jin Yin Hua. Moreover, administration of an herbal combination including two or more of Da Huang, Sheng Di Huang and Jin Yin Hua can be used as an effective, non-toxic bridge for weening a patient away from a established, if grueling, treatment regimen.

Contained within each of the one or more herbs of an herbal combination in accordance with embodiments set forth herein are several molecular constituents. An observed reduction of symptoms and suffering from a skin ailment such as psoriasis, eczema, or melanoma, or inflammatory disease, or an autoimmune disorder, or cancer owing to a treatment regimen including periodic doses of an herbal combination of Da Huang, Sheng Di Huang and Jin Yin Hua alone or in majority proportion combined with one or more additional herbs or in combination with one or more developed treatment regimens described above or below herein.

Methods of relieving side effects and/or treating a disease, and/or modulating inflammatory, hematological or immunological activity for the treatment of a disease, are also provided, including administering a combination of Sheng Di Huang, Da Huang and Jin Yin Hua, alone or in combination with or following administration of a regimen including one or more of the established regimens described above or below herein.

Advantageous effects of an established treatment regimen, such as any of those described herein, may be bolstered by administration of an herbal combination of Da Huang, Sheng Di Huang and Jin Yin Hua, alone or in further combinations with one or more additional herbs, active ingredients and/or by one or more buffer molecules or one or more molecules serving as some helpful vehicle for the active molecules. The chemistries and pharmacologies of several herbs and several molecules, molecular extracts, or molecular constituents are summarized below.

In certain embodiments, certain parts of one or more of the seven herbs are used such as the roots, stems, leaves, husks, branches, barks, sap, or kernels or combinations thereof. For example, for Da Huang at least the root may be used. For Sheng Di Huang, at least dried root tuber may be used. For Jin Yin Hua, at least dried flower may be used. For Mu Dan Pi, at least dried root bark may be used. For Di Gu Pi, at least dried root bark may be used. For Xian He Cao, at least dried aerial part of Agrimonia Pilosa Ledeb may be used. For Chun Gen Pi, at least dried bark of the root or stem may be used.

In certain embodiments, an established treatment regimen, such as any of those described herein, may be administered along with or before or after administration to a patient with a skin ailment such as psoriasis, eczema, or melanoma, or an inflammatory disease, or an autoOimmune disease, or cancer, an herbal combination of the two herbs Sheng Di Huang and Da Huang, or two or more of the three herbs including Jin Yin Hua, or combinations of the seven herbs including Mu Dan Pi, Di Gu Pi, Xian He Cao and/or Chun Gen Pi.

In certain embodiments, an established treatment regimen, such as any of those described herein, may be administered before, during and/or after administering any one or more of the three herbs Sheng Di Huang, Da Huang, and Jin Yin Hua, alone or along with one or more, or even all in certain embodiments, of one or more of Mu Dan Pi, Di Gu Pi, Xian He Cao and/or Chun Gen Pi and/or one or more of an additional eleven herbs including Zi Cao, or radix arnebiae (arnebia root) or radix lithospermi (gromwell root), Xuan Shen, or radix scrophulariae (figwort root), Shi Gao or gypsum fibrosum (gypsum), Bai Shao, or radix paeoniae alba (white peony root), Chi Shao or radix paeoniae rubra (red peony root), Hong Hua or flos carthami (safflower), Da Qing Ye or folium isatidis (woad leaf), Qing Dai or indigo naturalis (natural indigo), Bai Zhu or rhizoma atractylodis macrocephalae (largehead atractylodes rhizome), and Shi Wei or folium pyrrosiae (shearer's pyrrosia leaf), Rou Gui or cortex cinnamomi (cinnamomum bark).

In certain embodiments, an established treatment regimen, such as any of those described herein, may be administered before, during and/or after administering any one or more of the three herbs Sheng Di Huang, Da Huang, and Jin Yin Hua, alone or in combination with one or more of the other herbs described herein, and/or along with one or more molecules, molecular extracts or molecular compounds or constituents described herein, particularly in topical, subdermal and/or oral formulation for treating a skin ailment, inflammatory disease, antoimmune disorder or cancer.

### Da Huang

### Radix Et Rhizoma Rhei (Rhubarb)

### Chemistry

Da Huang contains free antraquinones, anthraquinone glycosides, and bianthrones. Among the free antraquinones contained within Da Huang are alizarin, aloe emodin, chrysophanol, citreorosein, emodin, laccaic acid D, physcion, and rhein. Among the anthraquinone glycosides contained within Da Huang are 1,8-dihydroxy-3-methylanthraquinone-1-O-β-D-glucoside (Palmatin), aloe emodin 1′-O-β-D-glucopyranoside, aloe emodin 1-O-β-D-glucopyranoside, chrysophanol 1-O-β-D-glucopyranoside (chrysophanein), chrysophanol 8-O-β-D-glucopyranoside, emodin 1-O-β-D-glucopyranoside, emodin 3-O-β-D-glucopyranoside (Glucoemodin), emodin 8-O-β-D-glucopyranoside, physcion 1-O-β-D-glucopyranoside, physcion 8-O-β-D-gentiobioside, physcionin, and rhein 1-O-β-D-glucopyranoside. Among the bianthrones contained in Da Huang are aloe emodin bianthrone, chrysophanol bianthrone, palmidins A-C, rheidins A-C, sennidins A, B and C and sennosides A-F.

Da Huang also includes other compounds including 2-(-2-hydroxy-propyl)-5-methyl-7-hydroxy-chromone, 2-(-2-hydroxypropyl)-methyl-7-hydroxy-chromanone, 2,5-dimethyl-7-hydroxychromone, 2-methyl-5-carboxymethyl-7-hydroxychromone, 3 napthalenes, 3,5,4′-trihydroxystilbene 4′-β-D-(2″-0-galloyl)-glucopyranoside, 3,5,4′-trihydroxystilbene 4′-O-β-D-(6″-O-galloyl)-glucopyranoside, 4′-O-methylpiceid, Rhapontin, Rheinosides A-D, Stilbene gakkates 3,5,4′-rtihydroxystilbene 4′-O-β-D-glucopyranoside, Stilbene piceid and Tannins.

### Pharmacology

### Purgative Effect of Da Huang

Da Huang is well known as a purgative agent. The active constituents are the combined anthraquinones, especially sennosides. The content of sennosides correlates with the purgative activity of rhubarb.

Table 1 illustrates the oral purgative ED₅₀ values of the anthraquinones

| | ED₅₀ | | ED₅₀ |
|---|---|---|---|
| Anthraquinones | (mg/kg) | Anthraquinones | (mg/kg) |
| Sennoside A | 13.5 | Aloe-emodin-8-glycoside | 71.6 |
| Sennoside B | 13.9 | Emodin monoglucoside | 103.6 |
| Sennoside C | 13.3 | Aloe-emodin | 59.6 |
| Sennoside D | 13.8 | Rhein | 97.5 |
| Sennoside E | 13.5 | Emodin | >500 |
| Sennoside F | 16.1 | Physcion | >500 |
| Rhein-8-glucoside | 20.0 | Chrydophanol | >500 |

Studies on the mechanisms of action found that sennosides act predominantly on the large intestine. The most potent purgative activity was obtained from the rhubarb extract or

Sennoside A by gastrical administration and from sennidin by intravenous route. Inhibition of the intestinal flora in mice with chloramphenicol significantly decreased the activity prosthetic sugar group of the anthraquinone glycosides prevented the anthrone from being oxidized before they are transported into the large intestine and hydrolysed by the bacterial enzyme into free sennidins. It was found that sennosides are hydrolysed by microbial β-glycosidase in a stepwise fashion to the corresponding sennidins via 8-monoglycosides. The resulting metabolites sennidins were further reduced, possibly by a reductase bound to cell membranes of intestinal bacteria, to rheinanthrone as the purgative principle.

Ligation of the junction of the large and small intestines failed to prevent anthraquinone glycoside producing a purgative effect in the large intestine. Oral administration of rhubarb started to produce effect 6-8 h later. These results suggest that there is also a large part of anthraquinone glycosides absorbed in the small intestine and transformed in the liver before they act on the pelvic plexus and produce peristalsis and purgation.

On the other hand, small doses (0.05-0.3 g by oral administration) rhubarb caused constipation because of its high content of tannins. The constipation effect can be prevented by decocting rhubarb together with Huang Lian (Rhizoma Coptidis). This is because the tannins and berberine, the main constituent of Huang Lian, form gelatinous precipitates during decoction.

### Antimicrobial Effect of Da Huang

Tested by mixing virus with dilutions of aloe emodin for 15 min at 37° C., herpes simplex virus type 2 and type 3, varicella-zoster virus, pseudorabies virus, influenza virus were inactivated. Electron microscopic examination of the virus demonstrated that the envelopes were partially disrupted, indicating that it is directly virucidal to enveloped viruses. Emodin and rhein showed antiviral activity against human cytomegalovirus (HCMV) strain AD-169. When tested against a ganciclovir-resistant strain of HCMV, the EC₅₀ value for rhein was superior to the value obtained for the AD-169 strain. The aqueous extract of R. palmatum inhibited hepatitis B virus (HBV) polymerase activity and to bind hepatitis B virus surface antigen (HBsAg). Intravenous dose of 50 mg/kg of the extract to duck hepatitis B virus (DHBV) carrier ducklings showed antiviral activity against DHBV using serum DHBV DNA level and DHBV DNA polymerase activity as antiviral indicators.

Rhubarb exhibited inhibition against staphylococci, Streptococcus hemolyticus, Corynebacterium diphtheriae, Bacillus subtilis, B. brucellosis, B. mycoides, B. smegatis, Mycobacterium graminis, Yersenia pestis, Salmonella tophi, S. paratyphi, Shigella dysenteriae and Neisseria gonorrhea. Staphylococci and Neisseria gonorrhea were most sensitive to the herb. The main antibacterial components were the anthraquinone derivatives with the structure of 1,9-dihydroxyanthraquinone. 3-Carboxyrhein, hydroxyaloe-emodin and hydroxyemodin showed the most potent antibacterial activity. The bacteriostatic concentrations of rhein, emodin and aloe-emodin against staphylococci, streptomycin, Corynebacterium diphtheria, Bacillus subtilis, B. anthracis and Salmonella tophy were mitochondrial respiratory chain of microorganisms. Respiration of Staphylococcus aureus was strongly inhibited by emodin, aloe-emodin and rhein. Rhein, emodin and rhein specifically interfered with the redox function NADH dehydrogenase.

The aqueous, ethanolic and ether extracs of rhubarb are also antifungal against many pathogenic fungi, including Achorion schoenleini, Trichopphyton concentricum, T. violaceum, T. gypsum, Nocardia asteroids, Epidermophyton flocosum and Sporotrichum schenckii. The decoction of rhubarb exhibited inhibition against the influenza virus. The minimal effective dose in chicken embryo in vitro and semi in vivo was 5 mg per embryo.

### Antineoplastic and Antimutagenic Effects of Da Huang

Intraperitoneal administration of 75 mg/kg of emodin produced a 45% inhibition against the mammary carcinoma of mice. The inhibition rates of 5 mg/kg of rhein and emodin against murine melanoma were 76% and 73%. Rhein, emodin and aloe-emodin inhibited murine leukemia P₃₈₈ in vivo, increasing the survival time and decreasing the ascites volume. They also inhibited Ehrlich ascites carcinoma, emodin was a strong inhibitor of respiration in Ehrlich ascites carcinoma cells, with an ED₅₀ of 20 μg/ml. Cellular respiration in leukemia L₁₂₁₀ cells was also inhibited. Palmatin, Cysophanein and physcionin also exhibited moderate cytotoxic activity against several types of carcinoma cells.

The extract of the herb from R. palmatum and emodin induced a dose-dependent decrease in the mutagenicity of benzo(a)pyrene [B(a)P,], 2-amino-3-methylimidazo(4,5-f) quinoline (IQ) and 3-amino-1-methyl-5H-pyrido(4,3-b)indole (trp-P-2) in Salmonella typhimurium TA98. It was further found that emodin reduced mutagencity of IQ by direct inhibition of the hepatic microsomal activation and not by interaction with proximate metabolites of IQ and/or by modification of DNA repair processes in the bacterial cell. Emodin also markedly decreased the mutagenicity of 1-nitropyrene (1-NP) in a dose dependant manner in Ames-microsomal test with S. typhimurium TA98 and the genotoxicity in SOS chromotest with E. coli PQ37. Furthermore, emodin significantly inhibited the formation of 1-NP DNA adducts in S. typhimurium TA98. The results suggest that emodin acts as a blocking and/or suppressing agent to reduce the direct-acting mutagenicity of 1-NP.

### Hemostatic Effect of Da Huang

Rhubarb is also in TCM as a hemastatic agent. The hemastatic activity has been proved experimentally and clinically. Rhubarb is effective for both external and internal hemorrhage. It was effective in the treatment and prevention of experimental gastric bleeding and ulcer formation in rats. Significant therapeutic effects of the powdered rhizome of R. palmatum in the treatment of gastrointestinal bleeding were also reported. It reduces coagulation time and the permeability and fragility of capillaries. It increases fibrinogen and promotes bone marrow to produce platelets.

### Immunosuppressive Effect of Da Huang

Emodin at 3×10⁻⁷−3×10⁻⁴ M dose-dependently suppressed the responses of human mononuclear cells to phytohemagglutinin and mixed lymphocyte reaction. It was further found that after exposure to emodin (10⁻⁶ M) the production of interleukin-1 (IL-1) and interleukin-2 (IL-2) and the expression of IL-2 receptor were all decreased, Emodin may be a new template for the development of better immunosuppressive agents for use against transplantation and autoimmune disease.

### Choleretic Effect of Da Huang

Rhubarb can stimulate construction of the gallbladder and relax Oddi's sphinctercan, thus promoting bile secretion. It also increases the contents of bilirubin and bile acid.

### Other Effects of Da Huang

Oral administration if emodin and rhein provoked marked diuretic, natriuretic and kaliuretic effects in rabbits. Oral administration of rhubarb increased urinary excretion of sodium and potassium, alkalizing urine to a pH value as high as 8.4. Rhubarb also inhibits the activites of pepsase, trypsase, pancreatic amylase and pancreatic lipase. It lowers blood pressure and blood cholesterol levels. Rabbits with fever induced by subcutaneous injection of the pneumococci responded with reduced temperature after oral administration of the decoction of rhubarb.

Intraperitoneal administration of 15 mg/kg of emodin exhibited antiinflammatory activity against carrageenin-induced pedal inflammation in rats. In the same dosage it also showed antiulcerative activity against pylorus-ligated, aspirin and immobilization stress-induced gastric ulcer in rats. It decreased acid and pepsin output and augmented mucus secretion in terms of total carbohydrate:protein ration in the gastric juice of aspirin treated pylorusligated rats, indicating that the antiulcerative effect of emodin may be due to this effect on gastric secretion.

### Mu Dan Pi

### Cortex Moutan (Paeony Bark)

### Chemistry

Mu Dan Pi contains Apiopaeonoside, Benzoyloxypaeoniflorin, Benzoylpaeoniflorin, Galloyloxypaeoniflorin, Galloypaeoniflorin, Mudanpiosides A-F, Oxypaeoniflorin, Paeconoside, Paeoniflorin, Paeonisuffral, Paeonisuffrone, Paeonol, Paeonolide, Pentagalloyglucose 1,2,3,4,6, and Suffruticosides A-E

### Pharmacology

### Antimicrobial Activity of Mu Dan Pi

The decoction of the root bark exhibited a strong antibacterial activity in vitro against the following bacteria: Bacillus subtilis, Escherichia coli, Salmonella typhi, S. paratyphi, Protues vulgaris, Pseudomonas aeruginosa. Staphylococcus aureus, Strephtococcus hemolyticus, Diplococcus pneumoniae and Vibrio cholerae. Paeonol was one of the antibacterial components; its MIC values were 1:2000 against Staphlococcus aureus, 1:1500 against Bacillus subtilis and Escherichia coli.

### Anti-Inflammatory Effect of Mu Dan Pi

The 70% methanolic extract of the root bark inhibited rat paw swelling induced by carrageenin. Paeonol was found active in inhibiting rat paw swelling induced by dextran, acetic acid or carrageenin. It also inhibited the increase of intra-abdominal capillary permeability of mice and cutaneous capillary permeability of guinea pigs caused by acetic acid or 5-HT. On the other hand, the water soluble fraction free from paeonol as well as the glycoside fraction also exhibited a significant inhibitory action on rat paw edema due to carrageenin. The water-soluble fraction was also effective in either preventing or treating adjuvant-induced arthritis in rats. The methanolic extract, the glycosidic fraction and paeonol inhibited blood platelet aggregation. ADP- or collagen-induced human plateet aggregation was inhibited by paeonol. The formation of thromboxan B₂ was also inhibited but the formation of 12-hydroxy-5,8,10,14-eocpsatetraenoic acid from C-arachidonate was stimulated. Besides, paeonol inhibited the formation of prostanoids such as prostaglandins and thromboxanes from C-arachidonate in rat peritoneal macrophages. Thus the anti-inflammatory action of the root bark was related to the inhibitory effects of paeonol on prostanoid synthesis.

### Hypotensive Effect of Mu Dan Pi

The blood pressure of dogs with essential or renal hypertension was significantly reduced after oral administration of 5 g/kg of the decoction of the root bark for 5 days and 10 g/kg for two more days. The blood pressure of dogs with renal hypertension was also lowered after oral of 10 g/kg of the decoction free paeonol for 10 days. Oral administration of 0.5-1.0 g/kg of paeonol also produced hypotensive action renal hypertensive dogs and rats.

### Effect on the CNS of Mu Dan Pi

Intraperitoneal or oral administration of paeonol decreased the spontaneous activity of mice, antagonized caffeine-induced hyperactivity and prolonged cyclobarital-induced sleep. At higher doses, paeonol caused disappearance of the righting reflex in mice. It also antagonized convulsions due to cardiazol, strychnine, nicotine and electric shock. Furthermore, paeonol was found to have antipyretic and analgesic activities. Paeonol decreased the body temperature of normal mice and the mice with typhoid and paratyphoid vaccine-induced fever. Oral administration of paeonol produced an analgesic effect against acetic acid-caused writhing and tail pain by pressing in mice.

### Effect on Obesity of Mu Dan Pi

The aqueous extract of the herb was given as drinking water at the concentration of 0.5% to (SLN×C₃H/He) F₁ obese mice between 3 and 32 weeks of age. The treatment resulted in a significant decline, particularly in males, in food intake and in the Lee index, An index of obesity, and furthermore an increase in glucose tolerance. No significant difference was observed between the experimental and the control groups in the serum free fatty acid levels. There was little difference between groups in the weights of heart, liver, lung, spleen and major endocrine organs in both sexes and in the pattern of oestrous cycles in females, There results indicate that the herb protects against obesity, especially in males, at least partly by a decrease in food intake and an increase in glucose metabolism.

### Other Effects of Mu Dan Pi

Paeonol exhibited anticholinergic and antihistaminic actions on isolated ileum of mice and guinea pigs. It also prevented stress ulcer in mice and inhibited gastric secretion in rats and spontaneous motility of rat uterus in situ. The extract of the root bark and paeonol were also of antimutagenic activity. They decreased the frequency of mutations induced by 4-nitroquinoline 1-oxide in Escherichia coli WP2s.

### Sheng Di Huang

### Radix Rehmanniae (Chinese Foxglove Root)

### Chemistry

Sheng Di Huang contains 4-(α-L-rhamnopyranosyloxy)-3-methoxybenzoylajugol, Aceutoside, Ajugol, Aucubin, Campesterol, Castanosides A and F, Catalpol, digoxin Echinacoside, E-feruloylajugol, Isoacetoside, Jioglutoside A and B, Jionosides A&B, Leonuride, Mannitol, Melittoside, p-courmaroylagujol, p-hydorxybenzoylajugol, Purpureaside C,

Rehmaglutins A, Rehmaionosides A-C, Rehmanniosides A-D, Rehmapicroside, Vanilloylajugol, Z-feruloylajugol, and β-sitosterol,

### Pharmacology

### Effects on Adrenocortical function and Cortisol Metabolism of Sheng Di Huang

The herb was able to stop the decrease of plasma corticosterone concentration due to administration of dexamethasone and prevent the adrenal cortex from atrophy. The corticosterone level in rabbits receiving dexamethasone was increased at week 4 and week 6 when the herb was concurrently applied. No morphological changes were observed in the pituitary gland and adrenal cortex of rabbits receiving concurrent treatment of dexamethasone and the herb. A single large dose (3 g/kg) of the root or together with two other herbs Zhi Mu (Rhizoma Anemarrhenae) and Licorice (0.9g/kg each) given orally antagonized the inhibitory effect of dexamethasone on the pituitary-adrenal system of rabbits, thereby increasing plasma corticosterone level.

This mixture also antagonized the inhibitory action of dexamethason on the early morning cortisol secretion peak in 12 normal subjects as tested in diurnal dexamethason suppression test. The crude extract (8 mg) of the root, when incubated with the liver sections of rabbits, protected cortisol from being reduced on the double bond between C₄ and C₅, and the ketone at C₃ and being degraded of the hydroxyl groups at C₁₇ and C₂₁, and the ketone at C₂₀, thus delaying the metabolism of cortisol in the liver. When the herb was used simultaneously with exogenous adrenocortical hormones, plasma cortisol could still be kept at a nearly normal level. The mechanism is believed to be a kind of competitive effect which influenced the binding of cortical hormone to the receptors and affected the uptake of corticosteroid hormone by the liver cells, thereby slowing down the catabolism of cortisol.

### Cardiovascular and Diuretic Effects of Sheng Di Huang

The effects of the herb on the heart were largely dependent on doses. There was no obvious cordial activity at 0.1 or 0.5% concentration. At 1% concentration, cordial effect was observed in the isolated perfused frog heart. This action was more obvious in weak heart. When concentrations were increased to 2-5%, the heart was inhibited. Its effects on blood pressure was also dose-dependent. In an experiment with perfuesd vessels, 1-3% of the extract caused vasoconstriction while at 5% vasodilation.

Intravenous injection of 2.5 ml of the root extract produced a diuretic effect in anesthetized dogs. This action may be related to the cordial and renal vasodilation activities.

### Effect on Blood Glucose of Sheng Di Huang

Results in animal experiments have been inconsistent on the effect of the herb on blood glucose. Hypoglycemic effects of alcoholic extract of the root at a subcutaneous dose of 2 g/kg or an oral dose of 4 g/kg in rabbits were reported by early researchers. The result obtained from the subcutaneous injection was more significant: the blood sugar was decreased to the lowest level 4 h after medication. Subcutaneous administration of the alcoholic extract to rabbits also inhibited the prolonged hyperglycemic effect elicited by carbohydrates from the root of Codonopsis pilosula (Dang Shen). Intramuscular administration of 20 g of the same extract also suppressed and prevented epinephrine-induced hyperglycemia in rabbits. Other studies, however, reported that the aqueous or alcoholic extract could only reduce the blood glucose of normal rabbits and was not effective in hyperglycemia due to epinephrine. But there were also reports that the herb had no effect on the normal blood glucose level of rabbits. The decoction or the ethanolic extract at 6 g/kg had no effect on the normal blood glucose measurements of rabbits within 6 h of medication. Subcutaneous administration of 20 g/kg of the same agents also failed to antagonize epinephrine-induced hyperglycemia in rats. More recently, a weak hypoglycemic activity of rehmannioside D in spontaneous diabetic mice was reported.

### Antiinflammatory and Immunosuppressive Effects of Sheng Di Huang

Formaldehyde-induced edema of rat paws subsided after oral administration of the decoction or alcoholic extract at the daily dose of 10 g/kg for 5 days. However, another report claimed that only the decoction and not the alcoholic extract had a significant anti-inflammatory activity. At the oral dose of 100 mg/kg, jionoside B and acetoside produced 36% and 18% suppression of hemolytic plaque forming cells in the spleens of mice. In the same test conditions, intraperitoneal dose of 30 mg/kg of cyclophosphamide had a 52.5% suppression.

### Hemostatic Effect of Sheng Di Huang

The coagulation time in rabbits was reduced after giving the yellow needle crystal obtained from the ethanolic extract of the root. Intraperitoneal administration of 10 g/kg of the decoction or ethanolic extract, or oral administration of the charred herb shortened the bleeding time in mice with tail wounds.

### Effect on Hemorheology of Sheng Di Huang

The effects of the herb on the hemorheology of inflammatory, thromosic and intact animals were examined. Oral administration of 200 mg/kg of the 50% ethanolic extract of the herb inhibited the reduction of fibrinolytic activity erythrocyte deformability, the decrease in erythrocyte counts and the increase in connective tissue of the thoracic artery in a chronic inflammatory model, adjuvant-induced arthritis. However, it was ineffective on the development of edema in the arthritic rats and on acute and chronic inflammation. In addition, the extract inhibited the reduction of erythrocyte deformability but not the decrease of coagulative factors in a thrombosic model, endotoxin-induced disseminated intravascular coagulation (DIC). It also exhibited a promoting effect on erythrocyte deformability and fibrinolytic activity in intact rats. There results suggests that oral administration of the extract can prevent an inducement of impediment in the peripheral microcirculation of various chronic diseases through the improvement of hemorheology.

### Other Effects of Sheng Di Huang

Antiradiation, antifungal and antihepatotoxic activities have also been observed with extract of the root in animals. The 100% injection solution of the root given intraperitoneally at 1 ml daily for 6 days mitigated platelet damage in rats caused by 600 rad of γ-irradiation. The aqueous extract of the root inhibited intro fungi mentagrophyton. Microsporum gypseum and M. audouini. The decoction of the root showed protective effect in mice against CCI-caused liver intoxication. Oral or intraperitoneal administration of 10 g/kg of the decoction or the alcoholic extract potentiated the hypnotic effect of pentobarbital sodium. Intraperitoneal dose of 20 g/kg of the decoction or the alcoholic extract protected mice from hypobaric hypoxia.

### Jin Yin Hua

### Flos Lonicerae (Honeysuckle Flower)

### Chemistry

Jin Yin Hua contains 2,6,6-trimethyl-2-vinyl-5-hydroxytetrahydropyran, Benzyl-alcohol, Carvacrol, Cis and trans-2-methyl-2-vinyl-5-(a-hydroxyisopropyl)-tetrahydrofuran, Epivogeloside, Eugenol, Geraniol, Hex-1-ene, Hex-3-en-1-ol,

Isochlogogenic acid b+c (two isomers of 3,4-dicaffeoyl quinic acid),

Isochlorogenic acid a (3,5-dicaffeoyl quinic acid), Linalool, Loganin, Lonicerin, Lonicerin, Luteolin, Methylcaffeate, Pinene, Saponins with oleanolic acid, Secologanin dimethylacetal, Secoxyloganin, sweroside, Vanillic acid, Venoterpin (gentialutine),

Vogeloside, α-terpineol, and β-phenylethyl alcohol

### Pharmacology

### Antimicrobial Activities of Jin Yin Hua

The extracts of both the flower and vine inhibited in vitro the following bacteria Staphylococcus aureus, Streptococcus hemolyticus, Escherichia coly, Shigella dysenteriae, Vibrio cholera, Salmonella typhi, S. oaratyphi, Diplococcus pneumoniae, Neisseria meningitides, Pseudomonas aeruginosa and Mycobacterium tuberculosis. It also potentiated the action of penicillin against the drug-resistant Staphylococcus aureus. Chlorogenic acid and isochlorogenic acid are believed to be the chief antibacterial components of the flower. Luteolin also showed an antibacterial activity. More than half of the mice receiving the LD dose of Pseudomonas aeruginosa or its endotoxin survived after given 7.5 g/kg of the injection solution of the flower by intraperitoneal administration. Intravenous administration of 6 g/kg of the distillate of the flower was also therapeutically effective in rabbits poisoned by the endotoxin of Pseudomonas aeruginosa.

Antifunal activity was observed with the aqueous extract of the flower against Microsporum ferrugineum and Nocardia asteroids. In the monolayer primary culture of the epithelial cells of human embryonic kidney, the decoction of the flower inhibited influenza virus, ECHO virus and herpes virus.

### Anti-Inflammatory Effect of Jin Yin Hua

Intraperitoneal administration of 0.25 g/kg of the flower inhibited carrageenin-induced paw edema in rats. Given twice a day at 8 g/kg for 6 days by Intraperitoneal injection, the extract of the flower showed antiexudative and antihyperplastic effects on croton oil-induced granuloma. Intraperitoneal administration of the injection solution increased the phagocytic activity of the inflammatory cells in mice. The decoction diluted to 1:1280 concentration was still able to promote leukocytic phagocytosis.

### Central Stimulant Effect of Jin Yin Hua

Oral administration of chlorogenic acid produced central stimulation in mice and rats in experiments using electric shock and revolving cage; the potency of the central stimulation was ⅙ that of caffeine. No addictive or synergistic action was observed when they were used together.

### Antilipemic Effect of Jin Yin Hua

Oral administration of 2.5 g/kg of the flower reduced the intestinal absorption of cholesterol and the plasma cholesterol level. In vitro experiments showed that the flower conjugated with cholesterol.

### Other Effects of Jin Yin Hua

Intraperitoneal administration of an aqueous-ethanolic extract of the flower of L. japonica to mice on day 8 after mating decreased pregnancy in the test animals dose-dependently. Intrauterine and intra-amniotic administration of the extract killed the fetuses in dogs and caused abortion in monkeys, respectively. The extract of the flower exhibited a mild prophylactic effect against experimental gastric ulcer in rats when given orally. Large oral doses of chlorogenic acid increased gastrointestinal peristalsis and promoted gastric and bile secretion. Chlorogenic acid had a stimulant effect on the isolated rat uterus.

### Di Gu Pi

### Cortex Lycii Radicis (Wolfberry Bark)

### Chemistry

Di Gu Pi contains 5α-stigmastan-3 6-dione, Betaine, Cinnamic acid, Kukoamine A, Linoleic acid, Lyciumamide, Melissic acid, Sugiol, and β-sitosterol

### Pharmacology

### Antipyretic Effect of Di Gu Pi

The aqueous or alcoholic extract of the herb, given orally or by injection, produced a significant antipyretic effect in rabbits with fever induced by pyrogen. Betaine was also active. A strong antipyretic effect was also exhibited by the aqueous fraction of the alcoholic extract at doses ranging from 0.75 to 7.5 g/kg equivalent of the crude drug. The precipitates of the extract from lead salt also showed comparable antipyretic activity to synthetic antipyretic analgesics.

### Hypoglycemic Effect of Di Gu Pi

Oral administration of the decoction of the herb decreased blood glucose level in rabbits by 14% in average; this action lasted for 7-8 h. The peak action was observed 3 to 4 h after administration. It was also less effective when give subcutaneously. Subcutaneous dose of 6 g/kg of the extract elicited a mean reduction of 14% of the blood glucose of rabbits after 1 h.

### Hypotensive and Anticholesterolemic Effect of Di Gu Pi

The decoction, macerate, tincture and injection solution of the herb produced a significant hypotensive effect in anesthetized dogs, cats, rabbits by intravenous or intramuscular administration and in anaesthetized rats by oral administration. Repeated intravenous administration at lower doses did not induce rapid tolerance. Intravenous injection of 0.375 g/kg of the injection solution resulted in sudden drop of blood pressure and death of anesthetized dogs. Bradycardia, prolongation of PR interval and depressed T wave in the ECG were observed. Kukoamine A induced hypotension in rats when given intravenously at a dose of 5 mg/kg.

Daily oral administration of 10 g/kg of the extract of the herb for 3 weeks decreased the serum cholesterol in rabbits by 36.9% with little effect on triglyceride.

### Antimicrobial Activity of Di Gu Pi

In the sensitivity test using the paper disc method, the decoction of the herb strongly inhibited Bacillus typhosus, Salmonella paratyphi A, and Shigella flexneri but was inactive against Staphylococcus aureus. It was a weak bacteriostatic against Mycobacterium tuberculosis. In the primary monolayer tissue culture of the embryonic renal cells, the decoction prevented the pathogenic changes in the cells due to Asian influenza virus A JK strain.

### Effect on the Uterus of Di Gu Pi

The 100% injection solution of the bark showed stimulation effects on normal rat uterus and isolated mouse uterus. The activity of 1 ml of the solution was comparable to that of 0.054 unit of pituitrin.

### Xian He Cao

### Herba Agrimoniae (Hairyvein Agrimonia Herb)

### Chemistry

Xian He Cao contains Agrimols A, B, C, D and E, Agrimoniin,

Agrimonolide, Agrimorphol, Apigenin-7-glucoside, Caffeic acid, Ellagic acid, Gallic acid, Luteolin-7-glucoside, Pendinculagin, Potentillin, and Quercetin

### Pharmacology

### Teniacidal Effect of Xian He Cao

The winter sprout of the herb is used in folk medicine to expel tenia and the active principle was found to be agrimophol. Agrimophol acts directly on the parasite. It inhibits the glycogenolysis, aerobic and anaerobic metabolism in the parasite.

The herb and agrimophol are also lethal to some other parasites, such as Trichomonas vaginalis, blood fluke and roundworm.

### Antibacterial Activities of Xian He Cao

Six compounds, luteolin-7-glucoside, apigenin-7glucoside. Quercetin, ellagic acid, caffeic acid and gallic acid, isolated from the herb were active against bacillary dysentery. Combination use of luteolin-7-glucoside and ellagic acid, apigenin-7-glucoside and apigenin-7-glucoside was more effective than the respective individual compounds.

### Antitumor Effect of Xian He Cao

Agrimoniin had antitumour activity when given as a pre- or posttreatment. A single dose of 10-30 mg/kg prolonged the life span of mice bearing MM₂ tumors or yielded cures when given intravenously or orally prior to or after tumor inoculation. Agrimoniin also inhibited the growth of MH-134 and Meth-A solid tumors in mice. It was strongly cytotoxic to MM₂ cells in vitro, but the activity was almost completely abolished by the addition of fetal calf serum to the culture. Intraperitoneal administration of agrimoniin increased the number of peripheral white blood cells and the proportion of monocytes. The antitumor activity of agrimoniin appeared to be due to its enhancement of the immune response.

### Cardiovascular Effect of Xian He Cao

Intravenous administration of the alcoholic extract of the herb increased blood pressure and stimulated respiration in anesthetized rabbits and dogs, but the alcohol-soluble fraction of the aqueous extract lowered blood pressure in rabbits. Perfused into the blood vessels of rabbit ear and frog hind limb, it caused vasoconstriction at low concentrations and vasodilation at high concentrations. The extract and agrimoniin increased the heart rate and cardiac contractility of frogs and toads. On the other hand, the alcohol-soluble fraction of the aqueous extract inhibited the isolated frog heart.

### Chun Gen Pi

### Cortex Ailanthi (Tree-Of-Heaven Bark)

### Chemistry

Chun Gen Pi contains 1-(1′,2′-dihydroxyethyl)-4-methoxy-β-caboline, 1-(2′-hydroxyethyl)-4-methoxy-β-carboline, 1-(2-hydroxy-1-methoxy)-ethyl-4-methoxy-β-carboline, 13(21)-dehydro-glaucarubinone, 13(21)-dehydroglaucarubolone, 1-acetyl-4-methoxy-β-carboline, 1-carbamoyl-β-carboline, 1-carbomethoxy-β-carboine, 1-hydroxycanthin-6-one, 1-methoxycanthin-6-one, 1-methoxy-canthin-6-one-3-oxide, 5-hydroxymethylcanthin-6-one, 6-methoxy-β-carboline-1-carboxylic methyl ester, Ailanthone, Ailantinols A and B, Amarolide, Amarolide 11-acetate, Canthin-6-one, Canthin-6-one-3-oxide, Chaparrinone, Chaparrolide, Glaucarubinone Quassinoids Δ¹³⁽¹⁸⁾-dehydro-glaucarubinone, Δ¹³⁽¹⁸⁾-dehydroglaucarubolone, Shinjudilactone, Shinjulactones A-N, and β-carboline-1-propionic acid.

### Pharmacology

Glaucarubinone and ailanthone showed amebicidal activity in vitro against the parasite Entamoeba histolytica. Some quassinoids markedly inhibited the growth of chloroquine-resistant Plamodium falciparum. Glaucarubinone produced complete inhibition at 0.0006 μg/ml. Ailanthone also showed potent antiulcer activity.

### Molecules and Herbal Ingredients

### Aloe Emodin

Aloe emodin has a molecular weight around 270.24 g/mol. As to its anti-cancer activity, aloe emodin exhibits cytotoxicity in SCC of tongue, cervix cancer cells; and apoptoticity through MAPK-JNK cascade in hepatoma cells. Aloe emodin also tends to induce P53 and apoptosis. In addition, the cytotoxicity of aloe emodin induces effects in melanoma, and gastric carcinoma. As to its anti-inflammatory activity, aloe emodin exhibits anti TNF and anti virality in enveloped viruses—HSV, PSV, VSV, and INF. Aloe emodin also decreases COX2 and INOS expression in inflammation, and increases IFN in JEV and EV71. Aloe emodin may also be ingested for its properties as a laxative. Use of aloe emodin can induce nausia, and intestinal contraction causing abdominal pain. Long use of anthraquinones can lead to kidney and liver damage. The half-life of aloe emodin is about 78 min.

### Chrysophanol

Chrysophanol has a molecular weight around 254.24 g/mol. As to its anti-cancer activity, chrysophanol exhibits necrosis in hepatic cancer cells, including ATP change and ROS cascade. Chrysophanic acid inhibits EGFR in colon cancer cells. As to its anti-inflammatory activity, chrysophanol can serve as an antiseptic, bactericide, candidicide, and/or cathartic. In addition, chrysophanol can be used as an anti-staph aurus and an anti-bacilus subtilis. Chrysophanol can be used to suppress the activation of NF-kB. and caspase-1 in LPS-stimulated macrophages. Chrysophanol is also an anti polio virus compound. Chrysophanol an be used as a hemostat and as a pesticide, and can be used in the treatment of menorrhagia, including bleeding following abortion, epistaxis, functional uterine bleeding and thrombocytopenia. Chrysophanol acts as a purgative, and can be used to converts aloe emodin through P450 in the liver. Emodin and chrysophanol may be ingested in combination as an anti cancer treatment agent. Chrysophanol has a half life of about 2.75 hours.

### Emodin

Also known as Rheum emodi, emodin has a molecular weight around 270.24 g/mol. As to its anti-cancer activities, emodin exhibits pro apoptoticity in prostate cancer cells through P53, and P21. Emodin increases ROS, and improves chemotherapy effect in prostate cells which are drug resistant. Emodin exhibits pro apoptoticity through inhibition of IL6 in myltiple myaloma. Emodin exhibits anti-matastaticity0 through integrins effect. Emodin decreases HER2 in breast cancer and improves chemotherapoitic effect. Emodin induces cytotoxis in tongue carcinoma, and inhibits NFkB and other pro inflammatory cytokines. As to its anti-inflammatory activities, emodin may be used as an anti ulcer agent through Hpylori destruction and change in gastric fluid. Emodin induces a stabilizing effect on atherosclerotic plaque in vessels, and improves insulin and glucose changes in type 2 diabetes. Emodin promotes anti plasmodium against malaria, and may be used as an immunosuppressive, pesticide, purgative, spasmolytic, styptic, vasoelaxant, and/or viricide. Emodin has a half-life of approximately 227 min and converts to two active metabolites through P450 in the liver.

### Rhein

Also know as cassic acid, Rhein has a molecular weight around 284.22 g/mol. As to its anti-cancer activity, rhein acts as an anti proliferative in hepatic carcinoma, breast cancer, SCC of lungs, and cervical cancer. Rhein improves taxol effect in breast cancer, and inhibits nasopharengeal carcinoma and EGFR. Rhein serves as a sinergist with mitomycin. Rhein exhibits cytotoxicity in tongue carcinoma. Rhein may be used for anti angiogenesis. As to its anti-inflammatory activity, rhein exhibits anti fibroticity, and promotes anti proliferation of hepatic cells through inhibition of TGFb 1. Rhein may be used as an anti oxidant. Rhein decreases IL1B, and IL18 proinflammatory cytokines. Rhein is also anti bacterial, and may be used against staph. Aurus. Rhein also increases sensitivity to ADH (drug). Rhein has a half-life of about 205 min (approx). Rhein is hydrophobic, and may be combined with lysin in order to be hydrophilic, as rhein lysinate.

### Chrysophanein

Chrysophanein exhibits significant in-vitro cytotoxic activity in cancer cell lines

### Rhapontin

Rhapontin has a molecular weight of around 420.41 g/mol. As to its anti-cancer activities, rhapontin induces apoptosis and suppresses KATO III cell-growth in stomach cancer. Rhapontin also provides protective effects on LDL and erythrocytes against oxidative damage. Rhapontin has a half life of about 23.5 minutes.

### Stilbene Piceid

Stilbene piceid has a molecular weight around 390.2 g/mol. As to its anti-cancer activities, stilbene piceid inhibits DNA synthesis in LLC cells. Stilbene piceid also has an inhibitory effect on lipoxygenase. Stilbene also exhibits antioxidant activity and inhibits alpha-glucosidase. Stilbene piceid also inhibits lipid peroxidation induced by ADP and NADPH in liver microsomes. Stilbene acts directly on smooth muscle to promote pulmonary artery relaxation.

### Tannin

Tannin has a molecular weight around 500-3000 g/mol. As to its anti-cancer activity, tannin suppresses the growth of MCF-7 breast cancer cells. Geraniin, a form of tannin separated from geranium, causes cell death through induction of apoptosis. Tannin exhibits different antiproliferative effects against cervical and colon cancer cells grown in vitro. In pomegranate, tannin modulates inflammatory cell signaling in colon cancer cells. Tannin promotes apoptosis through induction of p53 non-small cell lung cancer cells. As to its anti-inflammatory activity, tannin in tomato suppresses COX-2 expression. Tannin can be used as an in vitro antioxidant and/or antiplatelet and also as an anti-inflammatory due to its free radical scavenging effects. Tannin may be used for its antiviral, antibacterial and/or antiparasitic effects. Tannin can be used in the treatment of HFE hereditary hemochromatosis. Tannin is capable of reversing 6-hydroxydopamine induced toxicity. Tannin has a dental use, as well, as tannin-fluoride preparation reduces gingival inflammation around abutment teeth. A large intake of tannins may cause bowel irritation, kidney irritation, liver damage, irritation of the stomach and/or gastrointestinal pain. A correlation has been made between esophogeal or nasal cancer in humans and regular consumption of certain herbs with high tannin concentrations. Tannins inhibit the absorption of minerals such as iron which may, if prolonged, lead to anemia. Tannins are present in soil, plants, water, tea, wine, and fruit. Tannin has a half life of about 3.15 hours.

### Carvacrol

Carvacrol, also known as cymophenol, has a molecular weight around 150.217 g/mol. As to its anti-cancer activities, carvacrol promotes anti-tumor effects on human metastatic breast cancer cells, including MDA-MB 231. Carvacrol is a potent inhibitor of cell growth inHuman Non-Small Cell Lung cancer. Carvacrol also inhibits growth of myoblast cells even after activation of mutated N-ras oncogene. As to its anti-inflammatory activities, carvacrol activates PPAR and suppresses COX-2 inflammation. Carvacrol may be used for its antiproliferative and antiplatelet properties. Carvacrol is present in the essential oil of Origanum vulgare, oil of thyme, oil obtained from pepperwort, and wild bergamot. Carvacrol inhibits the growth of several bacteria strains, e.g. Escherichia coli and Bacillus cereus, and in pseudomonas aeruginosa, carvacrol disrupts the bacteria membrane. Carvacrol may be used for its antioxidant activity. Carvacrol has a half life of about 1.29 hours.

### Eugenol

Eugenol has a molecular weight of about 164.2 g/mol. Eugenol induces apoptosis in human colon cancer cells, and inhibits invasion and angiogenesis of gastric carcinogenesis induced by MNNG. Eugenol in honey significantly inhibits the growth of Ehrlich ascites carcinoma. Eugenol-related biphenyl (S)-6,6′-dibromo-dehydrodieugenol elicits specific antiproliferative activity on neuroectodermal tumour cells partially triggering apoptosis. Eugenol causes melanoma growth suppression through inhibition of E2F 1 transcriptional activity. Eugenol may also be used as an antiseptic. Eugenol also inhibits platelet aggregation induced by agonists, including collagen, ADP and calcium ionophore. Eugenol may be extracted from certain essential oils especially from clove oil, nutmeg, cinnamon, basil and bay leaf. Eugenol is also used in perfumeries, flavorings, essential oils and in medicine as a local antiseptic and anesthetic. Eugenol may be used for its antioxidative properties. Eugenol exhibits hepatotoxicity. An overdose of eugenol may induce convulsions, diarrhea, nausea, unconsciousness, dizziness, and/or rapid heartbeat. Eugenol can be allergenic. Eugenol may express carcinogenicity through oxidative DNA damage by its metabolite. Eugenol has a half-life of 1.975 hours, and under certain conditions, may have a half life up to 4 hours or even 18 hours.

### Geraniol

Geraniol has a molecular weight of about 154.25 g/mol. As to its anti-cancer activities, Geraniol promotes an antiproliferative mechanism in human pancreatic adenocarcinoma cells. Geraniol also promotes anti-proliferative and cell cycle regulatory effects in human breast cancer cells. Geraniol can cause a 2-fold reduction of thymidylate synthase and thymidine kinase expression in colon cancer cells. Geraniol, as a component of plant essential oils, sensitizes human colon cancer cells to 5-fluorouracil treatment. Geraniol suppresses pancreatic tumor growth without significantly affecting blood cholesterol levels. As to its anti-inflammatory activities, Geraniol diminishes the levels of inflammatory markers induced by pamidronate stimuli in vitro and in vivo. Geraniol also promotes inhibitory effects on nitric oxide and prostaglandin E₂ production in macrophages. Geraniol is a component of rose oil, palmarosa oil, and citronella oil, and small quantities of geraniol are in geranium and lemon, has a rose-like odor and is commonly used in perfumes. Geraniol can be used as effective plant-based mosquito repellent. Geraniol is found in cigarettes. As to biologic use, ion-exchange iontophoresis combined with geraniol is a highly effective transdermal delivery system. Geraniol suppresses Candida cell growth in the vagina and its local inflammation when combined with vaginal washing. Gernaiol is also allergenic. Geraniol has a half life of about 0.713 hours.

### Luteolin

Luteolin has a molecular weight of 286.24 g/mol. As to its anti-cancer activity, luteolin, particularly in combination with standard anticancer drugs such as cisplatin, serves as a HDAC inhibitor, e.g., for the treatment of lung cancer. Luteolin promotes synergistic/additive growth inhibitory effects and may be effective in chemoprevention treatment of head and neck and lung cancers. Luteolin induces G1 arrest in human nasopharyngeal carcinoma cells. Luteolin not only protects DNA from oxidative damage, but also increases repair activity in Caco-2 cells. A low concentration of Luteolin has little toxic effect on cancer cells, but such low concentrations can sensitize chemotherapeutic drugs in various cancer cell lines. Luteolin selectively inhibits chymotrypsin-like and trypsin-like proteasome catalytic activities in tumor cells. Luteolin inhibits invasion of prostate cancer PC3 cells through E-cadherin.

Luteolin is a PDE4 inhibitor and a general phosphodiesterase inhibitor, and an Interleukin 6 inhibitor. Luteolin inhibits inflammatory response and improves insulin sensitivity in the endothelium. Luteolin prevents LPS-induced TNF-α expression in cardiac myocytes through inhibiting NF-κB signaling pathway. Luteolin inhibits myelin basic protein-induced human mast cell activation and mast cell-dependent stimulation of Jurkat T cells. Luteolin inhibits cyclooxygenase-2 expression and scavenges reactive oxygen species.

Luteolin is found in leaves, but it is also seen in celery, thyme, dandelion, rinds, barks, clover blossom and ragweed pollen. Luteolin is useful in the prevention and treatment of skin photoaging. Luteolin inhibits microglia and alters hippocampal-dependent spatial working memory. Luteolin enhances insulin sensitivity via activation of PPARγ transcriptional activity in adipocytes. Luteolin can induce nausea, vomiting and gastric hypersecretion. Luteolin has a half life of about 1.2 hours.

### Saponins

As to the anti-cancer activities of saponins with oleanolic acid, achyranthoside H methyl ester, a novel oleanolic acid saponin derivative from Achyranthes fauriei roots, induces apoptosis in human breast cancer MCF-7 and MDA-MB-453 cells via a caspase activation pathway. Saponion with oleanolic acid exhibit insecticidal activity against the Mexican bean beetle larvae (Epilachna varivestis).

### Vnaillic Acid

Vanillic acid has a molecular weight of 168.14672 g/mol. Vanillic acid suppresses metastatic potential of human cancer cells through PI3K inhibition and decreases angiogenesis in vivo. Vanillic acid enhances the activity of human lymphocyte proliferation and secretion of IFN-gamma. Vanillic acid has a beneficial effect on DSS-induced ulcerative colitis, thereby manifesting its usefulness in the regulation of chronic intestinal inflammation. Phenolic compounds in mushroom Lentinula edodes (shiitake) are hepatoprotective through their suppression of immune-mediated liver inflammation. Vanillic acid is found in the root of Angelica sinensis, and in olive oil. Vanillic acid promotes reduced cellular tyrosinase activity, DOPA oxidase and melanin contents, as well as down-regulated expressions of melanocortin-1 receptor (MC1R), microphthalmia-associated transcription factor (MITF), tyrosinase, and tyrosinase-related proteins 2 (TRP-2) and TRP-1. Vanillic acid contributes to the prevention of the development of diabetic neuropathy by blocking the methylglyoxal-mediated intracellular glycation system. There exist an oxidized form of vanillin. Vanillic acis has a half life of about 10.552 hours.

### α-Terpineol

The anti-cancer activities of α-terpineol, or alpha-terpineol, are partly mediated through the suppression of NF-kappaB activation. α-terpineol exhibits antiproliferative effects on erythroleukemic K562 cells. α-terpineol inhibits gene expression of the IL-6 receptor. α-terpineol suppresses fMLP-, LPS- and PMA-stimulated superoxide production. α-terpineol is found in cajuput oil, pine oil, and petitgrain oil, and is a common ingredient in perfumes, cosmetics, and flavors and tea. α-terpineol demonstrates different degrees of growth inhibition against 15 different genera of oral bacteria. α-terpineol can cause postural hypotension in pine oil, and can cause eye irritation. There are three isomers, alpha-, beta-, and gamma-terpineol. Alpha-terpineol has a half life of about 1.245 hours.

### Aucubin

Aucubin has a molecular weight of 346.32978 g/mol.

1. Antiproliferative activity is through cell cycle arrest and apoptosis in human non-small cell lung cancer A549 cells.

2. research—DNA damage induced by topoisomerase I poisoning as one of the possible mechanisms by which aucubin have shown antitumoral activity.

3. research—cytotoxic activity against MCF7-breast adenocarcinoma, HeLa-cervix adenocarcinoma, A431-skin carcinoma of epithelial origin.

4. can obstruct H(2)O(2)-induced apoptosis by regulating of the expression of Bcl-2 and Bax, as well as suppression of caspases cascade activation.

Aucubin enhance the activity of human lymphocyte proliferation and secretion of IFN-gamma. Aucubin is found in the leaves of Aucuba japonica (Cornaceae), Eucommia ulmoides (Eucommiaceae), and Plantago asiatic (Plantaginaceae). Aucubin protects against liver damage induced by carbon tetrachloride or alpha-amanitin, particularly when dosed intra-peritoneally. Aucubin provides neuroprotection in primary diabetic encephalopathy. Aucubin treatment can lower blood glucose. Aucubin can produce an increase in the level of lipid peroxidation and a decrease in activities of antioxidant enzymes in liver and kidneys. Aucubin has a half life of about 42.5 minutes.

### Digoxin

Digoxin, also known as digitalis, has a molecular weight of 780.938 g/mol. Digoxin treatment can inhibit HIF-1alpha synthesis and block tumor growth. Digoxin induces apoptosis in a human acute T-cell lymphoblastic leukemia cell line. Digoxin can serve as a specific neuroblastoma growth inhibitor and an unspecific inhibitor of angiogenesis.

Digoxin is widely used in the treatment of various heart conditions, namely atrial fibrillation, atrial flutter and sometimes heart failure that generally cannot be controlled by other medication. Digoxin increases myocardial contractility, such that the heart rate is decreased, while blood pressure increases as stroke volume is increased, leading to increased tissue perfusion. Digoxin improves myocardial efficiency due to improved hemodynamics, and improves the ventricular function curve. Digoxin affects the kidney by increased renal blood flow and increased GFR. A mild diuretic effect is seen typically only in heart failure. Digoxin may cause AV junctional rhythm and ectopic beats (bigeminy) resulting in ventricular tachycardia and fibrillation.

Digoxin can induce loss of appetite, nausea, vomiting and diarrhea as the gastrointestinal motility increases. Other common effects of Digoxin are blurred vision, visual disturbances (yellow-green halos and problems with color perception), confusion, drowsiness, dizziness, insomnia, nightmares, agitation, and depression, as well as a higher acute sense of sensual activities. Less frequent adverse effects of digoxin (0.1%-1%) include: acute psychosis, delirium, amnesia, convulsions, shortened QRS complex, atrial or ventricular extrasystoles, paroxysmal atrial tachycardia with AV block, ventricular tachycardia or fibrillation, and heart block. Dangerous interactions can occur between digoxin and verapamil,amiodarone, erythromycin, and epinephrine. The efficacy of chemotherapeutic agent substrates of Pgp may be strongly reduced in patients taking digoxin. Digoxin treatment increases the risk of invasive breast cancer among postmenopausal women. Digoxin has a half-life around 36 hours.

### Isoacetoside

Isoacetoside, as an extract from Tecoma stans, exhibits a cytotoxic effect on human hepatocarcinoma cells (Hep-G2). Isoacetoside has a half life of about 3.7-6.4 hours.

### β-Stosterol

β-sitosterol, or beta-sitosterol, has a molecular weight around 414.71 g/mol. β-sitosterol may be used to treat prostatic carcinoma and breast cancer. β-sitosterol may have chemopreventive potential by virtue of its radical quenching ability in vitro, with minimal toxicity to normal cells. β-sitosterol also attenuates beta-catenin and PCNA expression, making it a potential anticancer drug for colon carcinogenesis. β-sitosterol significantly inhibits the growth, and induces the apoptosis, of SGC-7901 human stomach cancer cells in vitro. The decrease of the bcl-2/bax ratio and DNA damage may produce apoptosis induced by beta-sitosterol in SGC-7901 human stomach cancer cells. β-sitosterol enhances tamoxifen effectiveness on breast cancer cells by affecting ceramide metabolism. β-sitosterol has a proapoptotic effect that is mediated through the activation of ERK and the block of the PI3K/Akt signal pathway in MCA-102 cells. Therefore, beta-sitosterol has a strong potential as a therapeutic agent for preventing cancers such as fibrosarcoma. Beta-sitosterol is an effective apoptosis-promoting agent and that incorporation of more phytosterols in the diet may serve a preventive measure for breast cancer. An anti-microtubule characteristic of beta-sitosterol may contribute to the proliferation inhibition of SiHa cells in cervical cancer. β-sitosterol activates the sphingomyelin cycle and induces apoptosis in LNCaP human prostate cancer cells. β-sitosterol promotes anti-asthmatic actions that may be mediated by inhibiting the cellular responses and subsequent release/synthesis of Th2 cytokines. β-sitosterol may have therapeutic potential in allergic asthma.

β-sitosterol is found in Nigella sativa, pecans, Serenoa repens (saw palmetto), avocados, Curcurbita pepo (pumpkin seed), Pygeum africanum, cashew fruit, rice bran, wheat germ, corn oils, soybeans, sea-buckthorn, wolfberries, and Wrightia tinctoria.

β-sitosterol reduces blood levels of cholesterol, and is sometimes used in treating hypercholesterolemia. β-sitosterol may produce a positive effect on male hair loss in combination with Saw palmetto. β-sitosterol may play a major role in herbal therapy, especially in the treatment of benign prostatic hyperplasia. Beta-sitosterol is a naturally occurring phytosterol that may be used to cure atherosclerosis, diabetes, cancer, and inflammation and is also an antioxidant. β-sitosterol has a half life of about 0.966 hours.

By taking more than the recommended dose of f3-sitosterol, people may suffer from stomach upset, nausea, diarrhea, gas or constipation, impotence (also known as erectile dysfunction or ED), decreased sex drive. Beta-Sitosterol should be avoided during pregnancy and breast-feeding, since it is not proven to be benign with regard to potential effects on unborn and newborn children. β-Sitosterol is also not recommended for individuals with sitosterolemia, a rare inherited fat storage disease, because people with this condition have too much β-sitosterol and related fats in their system, taking β-sitosterol will only worsen this condition. High levels of β-sitosterol concentrations in blood have been correlated with increased severity of heart disease in men having previously suffered from heart attacks, and may cause allergy.

Half lives have been indicated for certain molecules. The half lives of molecules can vary from these, e.g. generally based on the herb growing and/or preparation conditions, how it is combined with other herbs of molecules in treatment, or based on patient characteristics and behaviors such as eating and drinking and physical activity, or on potency or other factors. Doses and dose periods may be determined based in part on the half lives. Typically, however, doses and dose periods will be determined based on characteristics of the patient, the patient's condition and the patient's history, as well as on the expertise and experience of the attending physician.

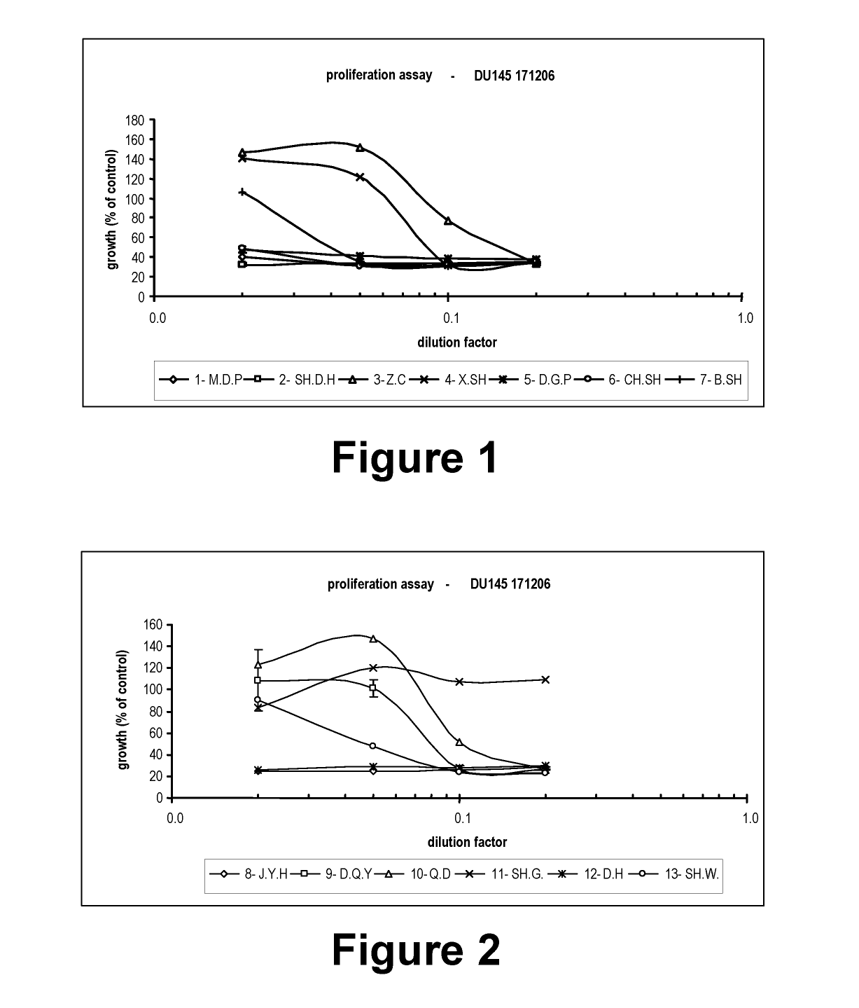

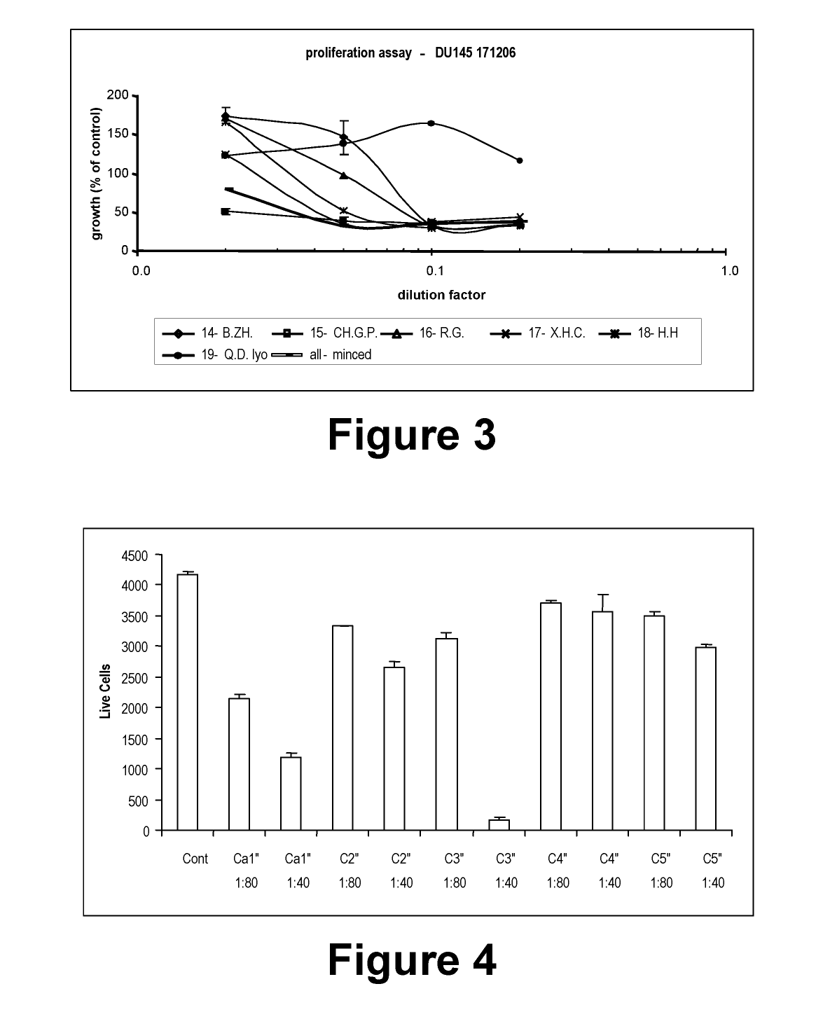

FIGS. 1-3 illustrate plots for each of the eighteen herbs of growth as a percentage of control versus dilution factor. FIGS. 1-3 illustrate that the three herbs Sheng Di Huang, Da Huang and Jin Yin Hua are most effective, while the four herbs Mu Dan Pi, Di Gu Pi, Xian He Cao and Chun Gen Pi are effective, and the eleven herbs Zi Cao, Xuan Shen, Shi Gao, Bai Shao, Chi Shao, Hong Hua, Da Qing Ye, Qing Dai, Bai Zhu, Shi Wei and Rou Gui are somewhat less effective.

FIG. 4 illustrates the effect on live cells of five combinations of herbs each in two concentrations 1:40 and 1:80. Cal includes a combination of all eighteen herbs, C2 includes a combination of Jin Yin Hua, Da Huang, Mu Dan Pi and Di Gu Pi, C3 includes a combination of Da Huang, Sheng Di Huang and Jin Yin Hua, C4 and C5 include combinations of the other eleven herbs (Zi Cao, Xuan Shen, Shi Gao, Bai Shao, Chi Shao, Hong Hua, Da Qing Ye, Qing Dai, Bai Zhu, Shi Wei and Rou Gui) of the eighteen herbs, i.e., not in combination with any of the seven herbs (Sheng Di Huang, Da Huang, Jin Yin Hua, Mu Dan Pi, Di Gu Pi, Xian He Cao and Chun Gen Pi). Specifically, C4 includes Shi Gao, Shi Wei, Bai Zhu, Rou Gui, Hong Hua and C5 includes Zi Cao, Xuan Shen, Chi Shao, Bai Shao, Da Qin Ye, and Qing Dai. FIG. 54 illustrates that combinations of all eighteen herbs, as well as combinations of the three herbs (Sheng Di Huang, Da Huang and Jin Yin Hua) are advantageously and synergistically most effective, while the combination C2 was less effective, and the combinations C4 and C5 were still less effective.

### EXAMPLE TREATMENTS AND MEDICINAL DOSAGE COMPOSITIONS

Treatment methods in accordance with certain embodiments may include administering periodic doses of an herbal combination of one or more herbs described herein, such as Da Huang, Sheng Di Huang or Jin Yin Hua, or combinations thereof, as a medicine or medicinal supplement, alone or in combination with a known or discovered treatment, for treating a patient with psoriasis, inflammatory disorders, autoimmune disorders, scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, proctitis, vasculitis, osteoarthritis, seborrheic dermatitis, acne, colitis, skin lesions, diabetes, hypertension, allergies, asthma, capuche sarcoma, autoimmune or inflammation related symptoms or disorders, dermatologic or cardiovascular conditions, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof.

A treatment method in accordance with certain embodiments may include administering periodic doses methotrexate, and/or another known treatment regimen described herein or as understood by those skilled in the art, along with an herbal combination of one or more herbs described herein, for treating a patient with psoriasis, eczema, melanoma, inflammation or a form of cancer that is known to be effectively treated with methotrexate such as cancer of the breast, skin, head and neck, or lung or rheumatoid arthritis, psoriasis or leukemia.

A treatment method in accordance with certain embodiments may include administering periodic doses of Pemetrexed, Pralatrexate, Methotrexate sodium, Pemetrexed Disodium or a folate analog metabolic inhibitor, alone or in combination with methotrexate, and/or another known treatment regimen described herein or as understood by those skilled in the art, along with an herbal combination of one or more of Da Huang, Sheng Di Huang, and Jin Yin Hua, and/or one or more of the other herbs described herein, for treating a patient with psoriasis, eczema, melanoma, inflammation or another inflammatory or autoimmune disease or form of cancer that is known to be effectively treated with methotrexate or another known treatment described herein or a discovered treatment such as for treating cancer of the breast, skin, bladder, head and neck, or lung, osteosarcoma, lymphoma, or trophoblastic neoplasms, or inflammation, or an autoimmune disease such as rheumatoid arthritis, juvenile dermatomyositis, psoriasis, psoriatic arthritis, lupus, sarcoidosis, capuche sarcoma, Crohn's disease, eczema and many forms of vasculitis, or leukemia or conditions for which immunosuppressive drugs are commonly used, or combinations thereof. p A treatment method in accordance with certain embodiments may include administering periodic doses of an herbal combination of one or more of Da Huang, Sheng Di Huang, and Jin Yin Hua, along with one or more of a class of drugs that are specifically understood for their effectiveness at treating auto immune diseases and/or for curbing bodily rejections after implantation or transplantation of an organ or bone marrow, including drugs that influence lymphocytes such as Azathioprine, Mycophenolate mofetil, Methotrexate and/or Cyclophosphoamide, and/or drugs that slow down meiosis of lymphocytes such as Cyclosporine, Tacrolimus, Sirolimus (Rapamycin) and/or drugs that neutralize cytokines such as Infliximab, Etanercept, Adalimunab and/or Anikra.

A treatment regimen may also include a known or discovered treatment and one or more of the herbs described herein in combination with approximately lug/m1-15 ug/ml of emodin, or approximately 5 ug/ml-10 ug/ml or more of emodin, alone or in combination with respectively 0.1 ug/ml-0.15 ug/ml of digoxin or 0.05 ug/ml-0.10 ug/ml or more of digoxin before, during and/or after administration of a known or discovered treatment regimen, such as those described herein or that may be understood by those skilled in the art or that may be discovered. Other combinations may be used in the treatment, including combining 5 ug/ml or more of emodin, alone or with at least approximately 0.10 ug/ml digoxin, or at least approximately 10 ug/ml emodin, alone or with at least approximately 0.10 ug/ml digoxin, or more than 5 ug/ml of emodin, alone or with at least approximately 0.05 ug/ml digoxin, or at least approximately 10 ug/ml emodin, alone or with at least approximately 0.05 ug/ml digoxin. Other combinations may be used and prescribed by physicians depending on factors such variances in weight, age, gender, family or patient history, or other characteristics specific to patients.

The treatment regimen may include once or twice daily doses, or several doses per day, or two or more weekly doses or otherwise. Doses may be taken more than once or twice a day, while the amounts of each dose would be determined according to the periodicity of the treatments.

### EXAMPLE DOSAGE COMPOSITIONS AND COMBINATION THERAPIES

Effective doses of an herbal combination of Da Huang, Sheng Di Huang and Jin Yin Hua have been demonstrated in mice between 1500 mg/kg and 500 mg/kg. Very low doses of 250 mg/kg may also be administered with modest effectiveness and elevated doses between 1500 mg/kg and 3000 mg/kg may be administered as tolerated with enhanced curative potential. This effective dose range in humans is approximately between 162.0 mg/kg and 40.50 mg/kg, while very low formulations of 20.25 mg/kg and high doses between 162 mg/kg and 324 mg/kg are also capable of formulation. The proportions of the three herbs have been shown to be effective at approximately one part Da Huang, one part Jin Yin Hua and between two and four parts Sheng Di Huang.

An elevated dose example for a 100 kg patient includes 2.4 grams Da Huang, 2.4 grams Jin Yin Hua and 7.2 grams Sheng Di Huang. For a 50 kg patient, an elevated dose example includes 1.2 grams Da Huang, 1.2 grams Jin Yin Hua and 3.6 grams Sheng Di Huang.

A medium dose example for a 100 kg patient includes 1.6 grams Da Huang, 1.6 grams Jin Yin Hua and 4.8 grams Sheng Di Huang. For a 50 kg patient, a medium dose example includes 0.8 grams Da Huang, 0.8 grams Jin Yin Hua and 2.4 grams Sheng Di Huang.

A low dose example for a 100 kg patient includes 0.8 grams Da Huang, 0.8 grams Jin Yin Hua and 2.4 grams Sheng Di Huang. For a 50 kg patient, a medium dose example includes 0.4 grams Da Huang, 0.4 grams Jin Yin Hua and 1.2 grams Sheng Di Huang.

A very low dose example for a 100 kg patient includes 0.4 grams Da Huang, 0.4 grams Jin Yin Hua and 1.2 grams Sheng Di Huang. For a 50 kg patient, a medium dose example includes 0.2 grams Da Huang, 0.2 grams Jin Yin Hua and 0.6 grams Sheng Di Huang.

An elevated dose example for a 100 kg patient includes 2.4-4.8 grams Da Huang, 2.4-4.8 grams Jin Yin Hua and 7.2-14.4 grams Sheng Di Huang. For a 50 kg patient, a high dose example includes 1.2-2.4 grams Da Huang, 1.2-2.4 grams Jin Yin Hua and 3.6-7.2 grams Sheng Di Huang.

In accordance with these examples, formulations may be prepared for and administered to 100 kg patients that include 0.4-4.8 grams Da Huang, 0.4-4.8 grams Jin Yin Hua and 1.2-14.4 grams Sheng Di Huang, and to 50 kg patients including 0.2-2.4 grams Da Huang, 0.2-2.4 grams Jin Yin Hua and 0.6-7.2 grams Sheng Di Huang. A moderate range example for 100 kg patients includes 0.8-2.4 grams Da Huang, 0.8-2.4 grams Jin Yin Hua and 2.4-7.2 grams Sheng Di Huang, and that for 50 kg patients includes 0.4-1.2 grams Da Huang, 0.4-1.2 grams Jin Yin Hua and 1.2-3.6 grams Sheng Di Huang. These example dosage compositions may be administered multiple times in a treatment regimen lasting a few days or weeks or even months at intervals of a few hours to daily, every other day or as needed.

In another example, a treatment regimen may include 1-10 gram daily doses of combinations of Da Huang, Sheng Di Huang and Jin Yin Hua. A treatment regimen may include 2-5 gram daily doses, or approximately 3 gram daily doses. In the 3.0 gram daily dose example, 1.5 gram doses may be administered twice daily, i.e., 1.5 grams twice a day. A total daily dose may be administered in the form of four (4) tablets, e.g., 2 tablets, twice a day, of 1.0 gram each containing 750 mg of a combination of Da Huang, Sheng Di Huang and Jin Yin Hua as well as 250 mg of excipients.

In another example, a psoriasis treatment regimen may include administration of combinations of two or more of Da Huang, Sheng Di Huang and Jin Yin Hua along with Ustekinumab (by Janssen) and/or with another IL-12 and/or IL-23 inhibitor.

In another example, a psoriasis treatment regimen may include administration of combinations of two or more of Da Huang, Sheng Di Huang and Jin Yin Hua along with Humira (by Abbott) and/or with another TNF alpha inhibitor.

In another example, a psoriasis treatment regimen may include administration of combinations of two or more of Da Huang, Sheng Di Huang and Jin Yin Hua along with Tofacitinib (by Pfizer) and/or with another JAK and/or STAT inhibitor.

In another example, a psoriasis treatment regimen may include administration of combinations of two or more of Da Huang, Sheng Di Huang and Jin Yin Hua along with Secukinumab (by Novartis) and/or another IL-17 inhibitor.

In another example, a psoriasis treatment regimen may include administration of combinations of two or more of Da Huang, Sheng Di Huang and Jin Yin Hua along with Otezla/Apremilast (by Celgene) and/or another PDE4 inhibitor.

In another example, a psoriasis treatment regimen may include administration of combinations of two or more of Da Huang, Sheng Di Huang and Jin Yin Hua along with Briakinumab and/or another p40 subunit of IL-12 and/or IL-23 and/or a humanized p40 monoclonal antibody.

In another example, a psoriasis treatment regimen may include administration of combinations of two or more of Da Huang, Sheng Di Huang and Jin Yin Hua along with Fezakinumab (by Pfizer) and/or another IL-22 inhibitor.

In another example, a psoriasis treatment regimen may include administration of combinations of two or more of Da Huang, Sheng Di Huang and Jin Yin Hua along with an IL-20 inhibitor.

In another example, a psoriasis treatment regimen may include administration of combinations of two or more of Da Huang, Sheng Di Huang and Jin Yin Hua along with an IL-23/p19 inhibitor.

In another example, a psoriasis treatment regimen may include administration of combinations of two or more of Da Huang, Sheng Di Huang and Jin Yin Hua along with a JAK3 inhibitor.

In another example, a psoriasis treatment regimen may include administration of combinations of two or more of Da Huang, Sheng Di Huang and Jin Yin Hua along with a Th1, Th17 and/or Th22 cell inhibitor.

In another example, a psoriasis treatment regimen may include administration of combinations of two or more of Da Huang, Sheng Di Huang and Jin Yin Hua along with an IFN gamma inhibitor.

In another example, a psoriasis treatment regimen may include administration of combinations of two or more of Da Huang, Sheng Di Huang and Jin Yin Hua along with an IL-17R, IL-19, IL-20, IL-22, sPLA2, NO (nitric oxide), VEGF, IL-24, key tyrosine and/or topoisomerase inhibitor.

In another example, a treatment regimen for IBS (irritable bowel syndrome) and/or colitis may include administration of combinations of two or more of Da Huang, Sheng Di Huang and Jin Yin Hua, alone, for example Da Huang and Sheng Di Huang as 2HX or including Jin Yin Hua as 3HX, or along with a TNF, IL-8, MIP-3-a, and/or ICAM-1 inhibitor, and/or one or more other inflammatory marker inhibitors. Moreover, a diagnostic or prognostic kit may include a test kit for measuring levels in gastrointestinal cells of one or more of TNF, IL-8, MIP-3-a, and/or ICAM-1 and/or one or more of, or a panel of several, inflammatory markers, and indicating an IBS and/or colitis diagnosis and/or prognosis for treatment.

Diagnostic and/or prognostic test kits are provided herein for each recited condition that include test kits for measuring one or more of, or a panel of several, markers associated with such recited condition, and indicators that provide diagnostics and/or prognostics for a patient. The prognostic and/or diagnostic kits may include one or more of Da Huang, Sheng Di Huang and Jin Yin Hua and/or one or more other herbs, molecules, and/or biologic, protein, molecule or receptor inhibitors or enablers, and/or treatments and/or medicines recited herein.

In another example, a psoriasis treatment regimen may include administration of combinations of two or more of Da Huang, Sheng Di Huang and Jin Yin Hua along with one or more of: a topical therapy such as Vitamin D, Calcipotriol, Corticosteroids, Dithranol, Retinoids, Tacrolimus, and/or Salicylic acid; a systemic therapy such as Methotrexate, Cyclosporine, Hydrea (hydroxyurea), and/or Retinoids; a phototherapy such as UV-B, Psoralen plus ultraviolet therapy and/or excimer laser; a combination therapy such as Methotrexate+Etanercept, Adalimumab (Humira®)+Calcipotriol+Betamethasone Dipropionate, and/or Alefacept+ultraviolet B (UVB) phototherapy, methotrexate, cyclosporine, and/or systemic retinoids; an herbal therapy such as Duzhong (Eucommia ulmoides Oliv.), Yerba mate (Ilex paraguariensis), Linseed oil, Fish oil, Indigo naturalis, Turmeric and/or Aloe Vera; and/or a biological and/or small molecule inhibitor and/or an enzyme inhibitor such as Denilukin diftitox, Efalizumab, Alefacept, Ustekinumab and/or Etanercept.

In another example, a rheumatoid arthritis (RA) treatment regimen may include administration of combinations of two or more of Da Huang, Sheng Di Huang and Jin Yin Hua along with Humira (by Abbott) and/or with another TNF alpha inhibitor.

In another example, a rheumatoid arthritis (RA) treatment regimen may include administration of combinations of two or more of Da Huang, Sheng Di Huang and Jin Yin Hua along with methotrexate.

In another example, a rheumatoid arthritis (RA) treatment regimen may include administration of combinations of two or more of Da Huang, Sheng Di Huang and Jin Yin Hua along with Enbrel (by Amgen & Pfizer) and/or another TNF alpha inhibitor approved for RA or for both psoriasis & RA.

In another example, a rheumatoid arthritis (RA) treatment regimen may include administration of combinations of two or more of Da Huang, Sheng Di Huang and Jin Yin Hua along with Remicade (by Janssen & Merck) and/or another TNF alpha inhibitor approved for RA or for two or more of psoriasis, RA and IBD (irritable bowel disease).

In another example, a rheumatoid arthritis (RA) treatment regimen may include administration of combinations of two or more of Da Huang, Sheng Di Huang and Jin Yin Hua along with Infliximab, Etanercept, Adalimumab, Anakinra, methotrexate, hydroxychloroquine, sulfasalazine and/or Leflunomide.

In another example, an Alzheimer's or Parkinson's treatment regimen may include administration of combinations of two or more of Da Huang, Sheng Di Huang and Jin Yin Hua, alone, or along with another approved Alzheimer's or Parkinson's treatment.

In another example, a rheumatoid arthritis (RA) treatment regimen may include administration of combinations of two or more of Da Huang, Sheng Di Huang and Jin Yin Hua along with an IL-17, IL-23, IL23 receptor and/or IL-23 axis inhibitor.

In another example, a RA, IBD and/or MS treatment regimen may include administration of combinations of two or more of Da Huang, Sheng Di Huang and Jin Yin Hua along with an IL-23 or IL-17 or TNF alpha inhibitor.

In another example, an RA, IBD, MS, Alzheimer's, Parkinson's, inflammatory colitis, osteoarthritis, psoriasis, eczema and/or dermatitis treatment may include administration of combinations of two or more of Da Huang, Sheng Di Huang and Jin Yin Hua along with an IL-1, IL-6 and/or IL-8 inhibitor.

In another example, an RA, IBD, MS, Alzheimer's, Parkinson's, inflammatory colitis, osteoarthritis, psoriasis, eczema and/or dermatitis treatment may include administration of combinations of two or more of Da Huang, Sheng Di Huang and Jin Yin Hua along with a BTK, SYK, ZAP-70, PI3KCD, AKT, HER-2, FLT-3, MAPK1, BRAF, and/or MEK1 inhibitor.

In another example, an approved treatment for AML, ALL, CIVIL, CLL and/or another leukemia or other cancer medication may be supplemented by administration of combinations of two or more of Da Huang, Sheng Di Huang and Jin Yin Hua.

In another example, leukemia or other cancer or other disease treatments that include a PD-1, CD279 and/or PD-L1 inhibitor may be supplemented by administration of combinations of two or more of Da Huang, Sheng Di Huang and Jin Yin Hua.

In another example, Desatinib and/or another LYN, BTK and/or ABL inhibitor, and/or Fostamatinib and/or another SYK, FLT3, KIT, LCK, JAL1, JAK3, PLC, AKT and/or BCR inhibitor, and/or Idelalisib (GS-1101) or another P13K delta inhibitor, and/or Ibrutinib and/or another BTK inhibitor and/or Gefitinib and/or another Zap 70 inhibitor, and/or Dasatinib and/or another Lyn inhibitor, or another approved treatment for CLL and/or another leukemia or other cancer medication may be supplemented by administration of combinations of two or more of Da Huang, Sheng Di Huang and Jin Yin Hua.

In another example, an Alzheimer's treatment may include administration of combinations of two or more of Da Huang, Sheng Di Huang and Jin Yin Hua along with a TNF-alpha, IL-6, IL-8, IL-12, GMCSF, and/or MCP-1 inhibitor.

In another example, a treatment may include administration of combinations of two or more of Da Huang, Sheng Di Huang and Jin Yin Hua along with a administration of combinations of two or more of Da Huang, Sheng Di Huang and Jin Yin Hua along with one or more kinase inhibitors, for example, an AKT1 (PKB alpha), ERBB2 (HER2), FLT3, MAPK1 (ERK2), PRKCA (PKC alpha), BRAF, BRAF V599E and/or MAP2K1 (MEK1) inhibitor.

Methods of preparing treatment medicines for psoriasis, eczema, melanoma, inflammation, leukemia or other cancer and/or an autoimmune disorder are also provided, including preparing a medicinal composition including methotrexate, or another known treatment described herein, or a discovered treatment, and an herbal combination of one or more of Sheng Di Huang, Da Huang, Jin Yin Hua, or one or more of the other herbs described herein and/or one or more molecules, molecular extracts or molecular compounds described herein.

### EXAMPLE FORMULATIONS

In one example formulation, a topical or oral formulation, or IV, subdermal, patch or other formulation described herein or understood to those skilled in the art may include 750 mg (milligrams) of a combination of Da Huang, Sheng Di Huang and Jin Yin Hua, e.g., 1:2:1 or 1:3:1 or 1:4:1, i.e., 187.5 mg of Da Huang, 375 mg of Sheng Di Huang and 187.5 mg of Jin Yin Hua or 150 mg of Da Huang, 450 mg of Sheng Di Huang and 150 mg of Jin Yin Hua or 125 mg of Da Huang, 500 mg of Sheng Di Huang and 125 mg of Jin Yin Hua or 100-200 mg Da Huang, 100-200 mg Jin Yin Hua and 300-600 mg Sheng Di Huang. The combination may include two herbs, e.g., 100-400 mg Da Huang or Jin Yin Hua and 300-600 mg of Sheng Di Huang or 100-650 mg of each of Da Huang and Jin Yin Hua. The combination may alternatively include 750 mg of one of Da Huang, Sheng Di Huang and Jin Yin Hua in certain embodiments including combinations with other herbs, molecules or medicines recited herein.

In another example formulation, a topical or oral formulation, or IV, subdermal, patch or other formulation described herein or understood to those skilled in the art may include 1-15% of a combination of Da Huang, Sheng Di Huang and Jin Yin Hua, e.g., 1:2:1 or 1:3:1 or 1:4:1, i.e., 0.25%-3.75% Da Huang, 0.5%-7.5% Sheng Di Huang and 0.25%-3.75% Jin Yin Hua or 0.2%-3% Da Huang, 0.6%-9% Sheng Di Huang and 0.2%-3% Jin Yin Hua or 0.15%-2.5% Da Huang, 0.5%-7.5% Sheng Di Huang and 0.15%-2.5% Jin Yin Hua or 2%, 2.5%, 3%, 4%, 5%, 6%, 7.5%, 9%, 10%, 11%, 13% 15% or 20% of a combination of Da Huang, Sheng Di Huang and Jin Yin Hua. The combination may include two herbs, e.g., 0.25%-5% Da Huang or Jin Yin Hua and 0.75%-10% Sheng Di Huang, or 0.5%-7.5% of each of Da Huang and Jin Yin Hua. The combination may alternatively include 1%-15% Da Huang, Sheng Di Huang or Jin Yin Hua alone, in certain embodiments particularly including combinations with other herbs, molecules or medicines recited herein. This example formulation may include 500 mg, 750 mg, 1000 mg or 1500 mg of a combination of Da Huang, Sheng Di Huang and Jin Yin Hua, e.g., 1:2:1 or 1:3:1 or 1:4:1, i.e., for the 750 mg example, 187.5 mg of Da Huang, 375 mg of Sheng Di Huang and 187.5 mg of Jin Yin Hua or 150 mg of Da Huang, 450 mg of Sheng Di Huang and 150 mg of Jin Yin Hua or 125 mg of Da Huang, 500 mg of Sheng Di Huang and 125 mg of Jin Yin Hua or 100-200 mg Da Huang, 100-200 mg Jin Yin Hua and 300-600 mg Sheng Di Huang.

In addition to the above active herbal combination ingredient, a medicinal tablet composition is provided for example for oral administration that may include one or more of the following further ingredients: 125 mg of MCC (Avicel), 20 mg of talc powder, 30 mg of aerosol 200, 10 mg of croscarmellose sodium and/or 30 mg of PVPK 30. This example may also include one or more of the following lubrication ingredients: 20 mg of croscarmellose sodium, 5 mg of aerosol 200, 20 mg of talc powder and/or 10 mg of magnesium sterate.

A medicinal cream is also provided for topical administration that includes the above active herbal combination ingredient, in an example concentration of 2.5%, and one or more of the following further ingredients: 2% sesame oil or sesamum indicum (sesame) seed oil, 4% cetostearyl alcohol or cetearyl alcohol, 5% arlacel 165 or glyceryl stearate or PEG-100 stearate, 3% light liquid paraffin or mineral oil, 2% cresmer wax EW or ceteareth-20 or cetearyl alcohol, 3% stearyl stearate, 0.2% butyl hydroxyl toluene or BHT, 4.2% propylene glycol, 0.2%potassium sorbate, 0.2% sodium benzoate, 5% glycerol or glycerin, 0.1%sandalwood oil or santalum album (sandalwood) oil, and 68.6% water.

A medicinal cream is also provided for topical administration that includes the above active herbal combination ingredient, in aqueous extract and in an example concentration of 2.5%, and one or more of the following further ingredients: till oil, cetostearyl alcohol, arlacel 165, light liquid paraffin, cresmer wax EW, stearyl stearate, butyl hydroxyl toluene, propylene glycol, sodium methyl paraben, sodium propyl paraben, glycerol, sandalwood oil. A cream placebo may be formulated for clinical trials that substitutes F-24 chocolate brown color TAS and brilliant blue color for the above active herbal combination ingredient.

### Preparation of Topical, Oral of Subdermal Medicine

Treatments described herein may be prepared for topical use for treatment of melanoma, eczema, dermatisis, BCC (basal cyr carcinoma) and inflammatory skin diseases like Psoriasis. For example, combinations of herbs and/or herbal extracts as described herein may be prepared as a cream to apply onto the skin. Another known or discovered treatment may be included or may be administered separately before, during or after the herbal treatment. The other known or discovered treatment, along with herbal combinations and/or herbal extract combinations described herein may be injected to infected areas of the skin of a patient using a syringe. An example method for preparation of an external cream in accordance with certain embodiments is provided below.

First, two of more of the herbs may be cooked, for example, as described elsewhere herein or as may be understood or determined by those skilled in the art. A cream is then prepared that may be somewhat more of less than half herbs and half cream, e.g., a 25%-75% liquid of herbs in 1:1 ratio and 25%-75% cream may be used. The herbs can also be prepared as a tincture, e.g., soaking the herbs in alcohol for a period of time such as 2 weeks in a ratio of 1:3, for example. This herbal liquid can then be mixed with the cream in the same way as described above.

The herbs may be prepared for cooking by grinding and/or homogenizing the herbs. Grinding may be achieved using a Dyno-mill run once to obtain, e.g., 250 nm particle sizes or multiple times down to, e.g., 150 nm particle sizes. A high pressure homogenizer may be used, whereby the mixture is pushed through a filter, e.g., a 0.2 μm filter. Sonication or ultrasound may also be used.

Straining may be performed to get an aqueous extract. Lyophilization or freeze drying may be performed to prepare the mixture for in vitro use. Solubilization may be performed along with selection of a concentration of the extract. A cream, tablet, capsule, nano-lipid carrier, nanogel, or nano-chrystals, or nano-particulates may be formulated for administration to human or animal patients.

Treatments described herein may also be effective against immunodeficiency diseases such as HIV and AIDS, as well as other conditions affecting or caused by disorders of the immune system. Herbal combinations of one or more of Da Huang, Sheng Di Huang and Jin Yin Hua and/or one or more other herbs or molecules described herein may be administered as a nutritional supplement or as a supplement to an exercise regimen or as an energy supplement or as a pain relief supplement or as a diuretic or sleep aid. NSAIDs, such as ibuprofen, naproxen and aspirin, other non-steroidal anti-inflammatories, acetaminophen, and/or steroidal anti-inflammatories may be combined with an herbal combination of one or more of Da Huang, Sheng Di Huang and Jin Yin Hua and/or one or more other herbs or molecules described herein, with or without administration of a known or discovered treatment, before, during or after the herbal treatment, to treat inflammation or other ailments that are commonly treatable with NSAIDs, including chronic pain. Formulations may be prepared for oral or topical administration as long release, lipidized dosage compositions or as short release non-lipidized formulas.

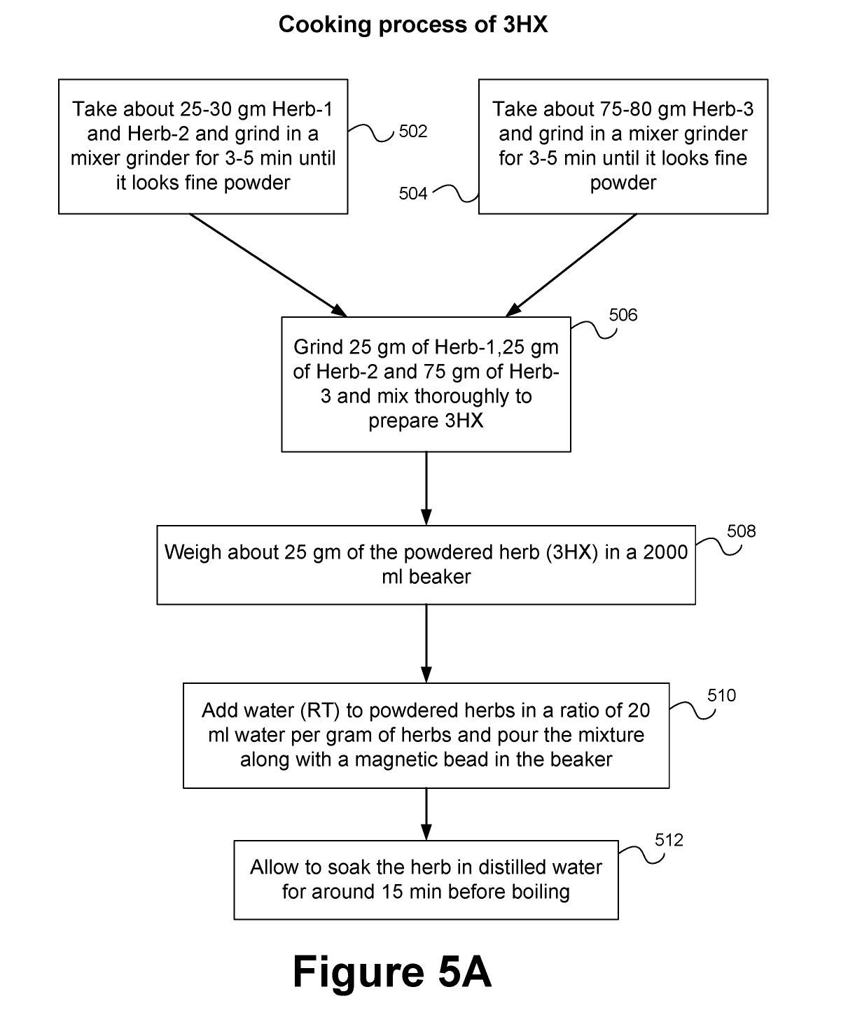

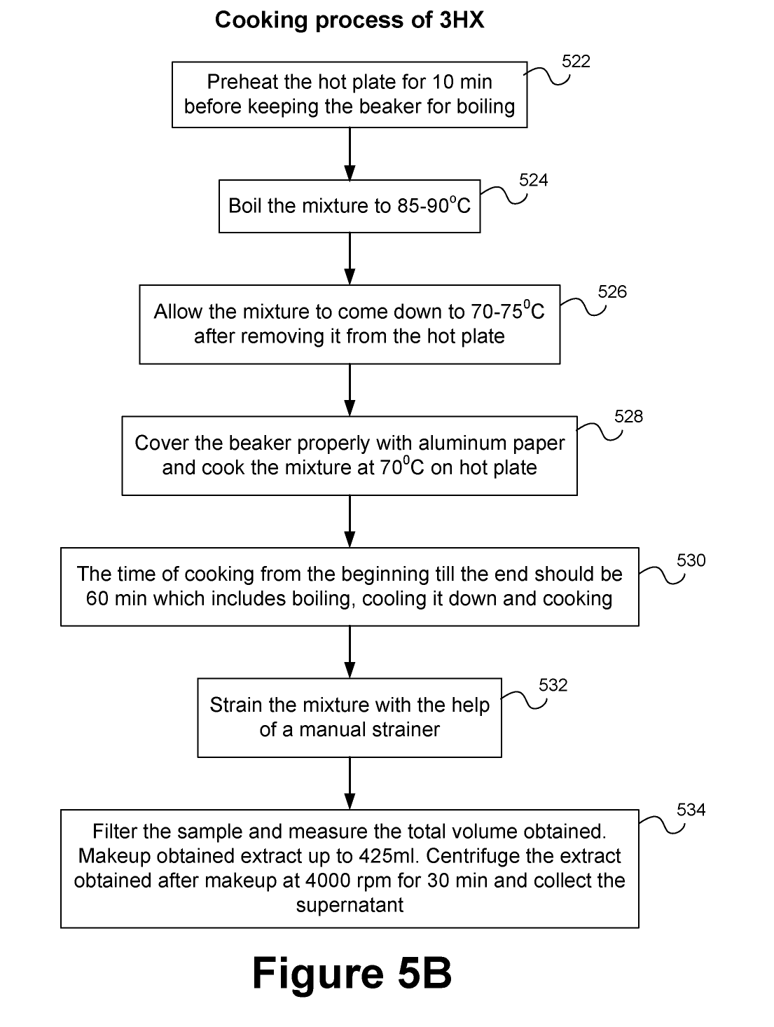

A cooking process may be performed as in the example of FIGS. 5A-5B. Referring to FIG. 5A, a step 502 in a cooking process may include taking about 25-30 grams of a first herb, e.g., da Huang, and a second herb, e.g., Jin Yin Hua, and grinding in a mixer grinder for three to five minutes or until a fine powder has been mixed and ground. A step 504 in the process may include taking about 75-80 grams of a third herb, e.g., Sheng di Huang, and grinding in a mixer grinder for three to five minutes or until a fine powder has been mixed and ground.

A step 506 in the cooking process may further include grinding 25 grams of the first herb, e.g., da Huang, 25 grams of the second herb, e.g., Jin Yin Hua, and 75 grams of the third herb, e.g., Sheng di Huang, and mixing thoroughly to prepare a three herb combination (“3HX”).

A next step 508 may include weighing about 25 grams of the powdered three herb combination 3HX in a 2000 ml beaker or other suitable container. A next step 510 may include adding water (RT) to the 25 grams of powdered three herb combination 3HX in a ratio of about 20 ml water per gram of powdered three herb combination and pouring the mixture along with a magnetic bead into the beaker. A next step 512 may include allowing the aqueous three herb combination to soak in distilled water for around 15 minutes before boiling.

Referring now to FIG. 5B, a hot plate may be pre-heated, for 10 minutes, e.g., before keeping the beaker for boiling, as indicated at step 522. A step 524 may then include boiling the mixture to 85-90° C. A step 526 may include allowing the mixture to come down to 70-75° C. after removing the mixture from the hot plate. A step 528 may include covering the beaker properly with aluminum paper and cooking the mixture at 70° C. on a hot plate. A time of cooking in accordance with the example of step 530 from the beginning to the end should be around 60 minutes which includes boiling, cooling and cooking.

A next step 532 may include straining the mixture with the help of a manual strainer. A step 534 may involve filtering the sample and measuring the total volume obtained, as well as making up an obtained extract up to 425 ml, centrifuging the extract obtained after making up at 4000 rpm for 30 minutes and collecting the supernatant.

A cream or lotion or shampoo or ointment or gel or patch or other topical formulation may be prepared. Also, a pill may be prepared in lipidized or non-lipidized form, and coated or uncoated for extended release, timed release, sustained release, modified release, immediate release, quick release, delayed release, controlled release, controlled delivery, long-acting or sustained action. Also, an IV or subdermal injection fluid may be formulated.

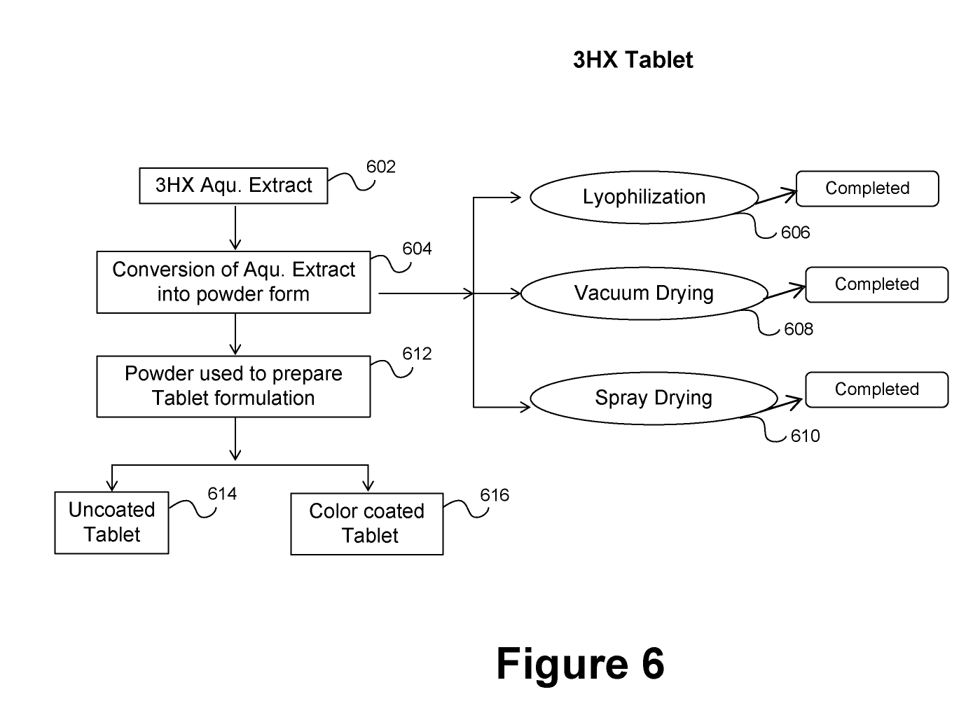

In the example of FIG. 6, an aqueous extract, e.g., including a herein-described one, two or three (or more) herb combination is prepared, e.g., in accordance with the example of FIGS. 5A-5B, or otherwise prepared, provided or acquired at step 602. A step 604 may include converting the aqueous extract into powder form. The converting into powder form of step 604 may include one or more of lyophilization or freeze drying 606, vacuum drying 608 and/or spray drying 610. The drying may include heating or proximate hygroscopic disposition or spinning or otherwise as may understood by those skilled in the art.

At step 612, the lyophilized or otherwise dried one, two or three (or more) herb powder may be used to formulate a tablet, pill, capsule or other orally-administered formulation. Such orally-administered formulation may be uncoated 614 or color coated 616. Batch sizes may be in multiples of one or more thousand.

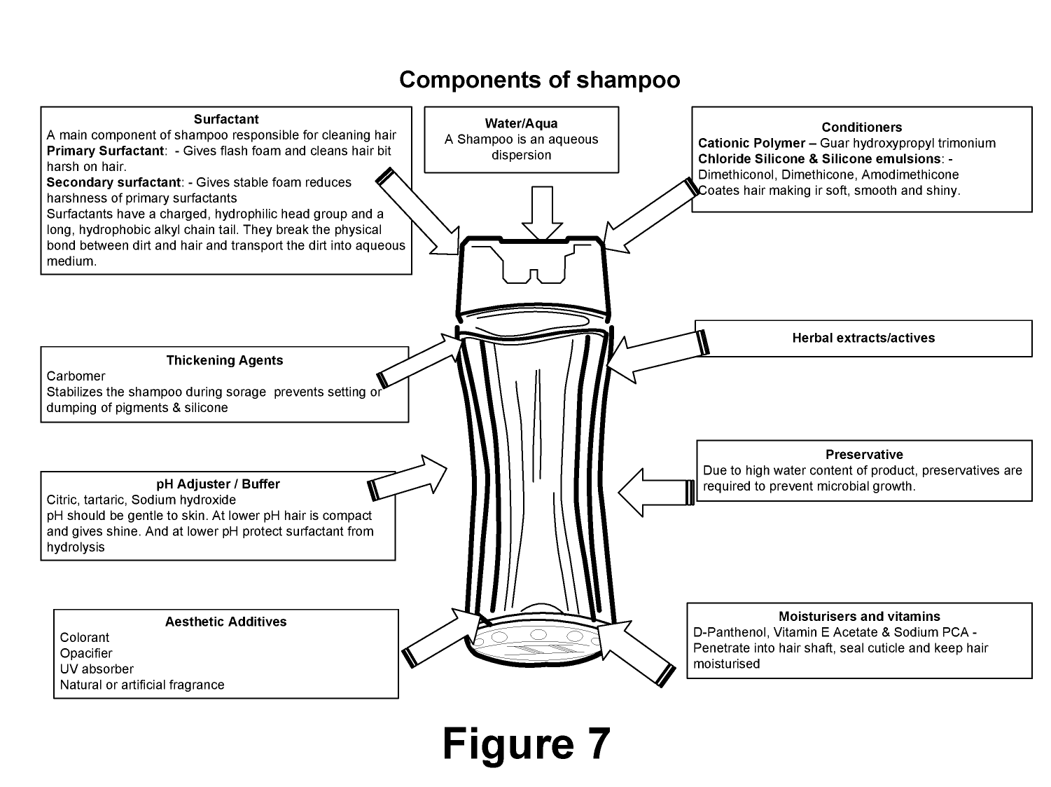

Referring to FIG. 7, an example of a shampoo in accordance with certain embodiments is illustrated as including nine generalized components. These nine components include a surfactant, a thickening agent, a pH adjuster/buffer, an aesthetic additive, water, a conditioner, one or more active herbs or herbal extracts or molecules, a preservative and moisturizers/vitamins. Shampoos in accordance with various alternative formulations may include fewer than all of these nine components and they may include other active or inactive components known to those skilled in the art as having some advantage when included in a shampoo formula or in a medicinal combination for treating a skin condition such as psoriasis, eczema, melanoma, or dermatitis or hair loss or another head or scalp disorder or ailment.

A shampoo in accordance with certain embodiments includes one or more surfactants that may be known or discovered as being advantageous for cleaning hair with a shampoo. A primary surfactant may be included to provide flash foam for cleaning the hair by removing dirt and other impurities. A secondary surfactant may be included to provide stable foam and to reduce the harshness of the primary surfactant. A surfactant may be used that includes a charged, hydrophilic head group and a long, hydrophobic alkyl chain tail. Surfactants are configured to break molecular bonds between dirt and hair and to transport the dirt into an aqueous medium to be rinsed free from the hair and scalp. Examples of surfactants that may be contained in a shampoo in accordance with certain embodiments include sodium laureth sulphate, ammonium laureth sulfate, and sodium cocoyl isethionate. Examples of co-surfactants include cocamide MEA and cocoamidopropyl betaine.

A shampoo in accordance with certain embodiments may include a thickening or suspending agent. Examples of thickening or suspending agents that may be contained in accordance with certain embodiments include carbomer and PEG 150 distearate. The thickening agent may be included to stabilize the shampoo during storage and/or to prevent the setting or dumping of pigments and silicone.

A pH adjuster or buffer may be included in a shampoo in accordance with certain embodiments. An example of a pH adjuster or buffer includes citric acid, tartaric and sodium hydroxide. The pH adjuster or buffer is configured to cause the shampoo to be gentle to the skin. A lower pH may cause hair to be compact and to shine and to protect the surfactant from hydrolysis, and as such, the pH adjuster or buffer may serve to lower the pH of the shampoo. However, alternative embodiments include pH adjusters that serve to raise the pH of a shampoo that contains an herbal formula that exhibits an exceptionally low pH.

An aesthetic additive may be included in a shampoo in accordance with certain embodiments. Examples of aesthetic additives include colorants, opacifiers, UV absorbers, perfumes and natural and artificial fragrances.

One or more conditioners may be included in a shampoo in accordance with certain embodiments. The one or more conditioners may include a cationic polymer such as guar hydroxypropyl trimonium chloride. The one or more conditions may include silicone and/or a silicone emulsion such as dimethiconol, dimethicone, or amodimethicone. The silicone and/or silicone emulsion may serve to coat the hair and cause the hair to become soft, smooth and shiny.

A shampoo in accordance with certain embodiments includes one or more active herbs or herbal extracts or emotives that are described in several examples herein. These one or more active herbs or herbal extracts serve to promote treatment of certain hair and scalp conditions such as psoriasis, eczema, dermatitis, melanoma, hair loss and other hair or scalp conditions described herein or understood by those skilled in the art.

A preservative may be included in a shampoo in accordance with certain embodiments. The preservative may be configured to prevent microbial growth. Examples of preservatives that may be contained in a shampoo in accordance with certain embodiments include paraben free, formaldehyde donor free and halogenated free.

A moisturizer and/or one or more vitamins may be included in a shampoo in accordance with certain embodiments. Examples include combinations of D-Panthenol, vitamin E acetate, sodium PCA, glycerine and one or more amino acids. The moisturizer and/or vitamins may be configured to penetrate into hair shaft, seal cuticles and keep hair moisturized.

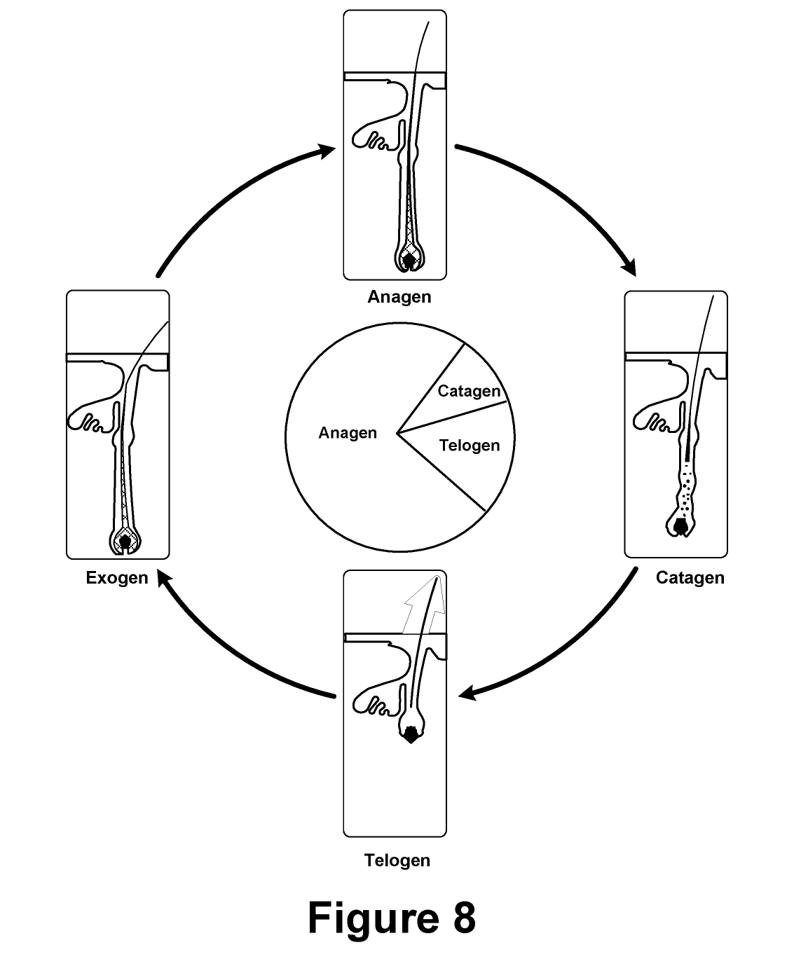

A shampoo in accordance with certain embodiments may include a hydro-alcoholic hair serum. Referring to FIG. 8, a hair growth cycle includes exogen, anagen, catagen and tetogen phases. The anagen phase involves active hair growth, whereby hair follicles regenerate and generate pigmented hair shafts. The telogen phase is a resting phase. The catagen phase involves cessation of hair growth and pigmentation, and release of papilla from the bulb. A hydro-alcoholic hair serum may be configured as a concentrated product that is typically left on the hair for a more extended duration than an ordinary shampoo with a typical shower routine. The hydro-alcoholic hair serum may be configured to be light and non-sticky on the scalp and as a non-irritant, to be light and non-sticky on the hair, to have little or no effect on hair volume, to strengthen scalp and DPC, to provide keratinization and collagen synthesis, to promote hair growth or to control hair fall, or combinations thereof. For example, the attributes of a hydro-alcoholic hair serum in accordance with certain embodiments may assist or promote treatment of psoriasis, seborrhea dandruff or hair fall. A hydro-alcoholic hair serum in accordance with certain embodiments may include water, alcohol, humectant, solubilizer, water-based polymer, scalp conditioner, niacinamide, caffeine and panthenol. The ratio of alcohol, water and solubilizer may be adjusted depending of the solubilization power of the active herbal treatment composition.

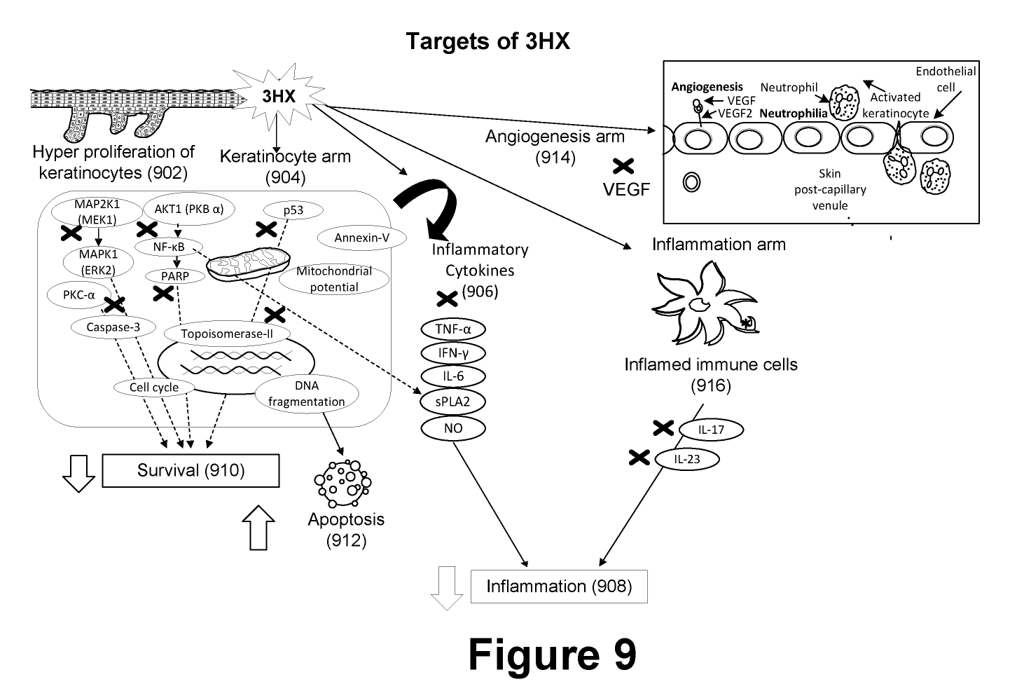

FIG. 9 schematically illustrates certain targets of medicinal compositions including combinations of Da Huang, Sheng Di Huang and Jin Yin Hua (3HX), in accordance with certain embodiments. FIG. 9 illustrates a hyper proliferation of keratinocytes 902, e.g., as may be present in a patient suffering with an inflammatory skin condition, or melanoma, or an autoimmune or other disorder involving the skin, or combinations thereof. 3HX may be administered to the patient to target keratinocyte-related activities 904, e.g., to inhibit MAP2K1 (MEK1) and MAPK1 (ERK2), AKT1 (PKB alpha) and NF-kB, p53, PKC alpha, PARP, topoisomerase-II, e.g., influencing Annexin-V, mitochondrial potential leading to inflammatory cytokines 906 and inflammation 908, Caspase-3 and/or cell cycles toward keratinocyte survival 910 and/or DNA fragmentation toward apoptosis 912.

FIG. 9 also illustrates that 3HX may be administered to a patient to target inflammatory cytokine activity 906 such as that of TNF alpha, IFN gamma, IL-6, sPLA2 and/or NO that may otherwise lead to inflammation 908.

FIG. 9 also illustrates that 3HX may be administered to a patient to target angiogenesis activity 914 such as that of VEGF, VEGFR2 and/or neutrophil that may activate keratinocytes and influence endothelial cells and/or skin post-capillary venules.

FIG. 9 also illustrates that 3HX may be administered to a patient to target inflammation 908 and particularly inflamed immune cell activity 916 such as that of IL-17 and/or IL-23 that may otherwise lead to inflammation.

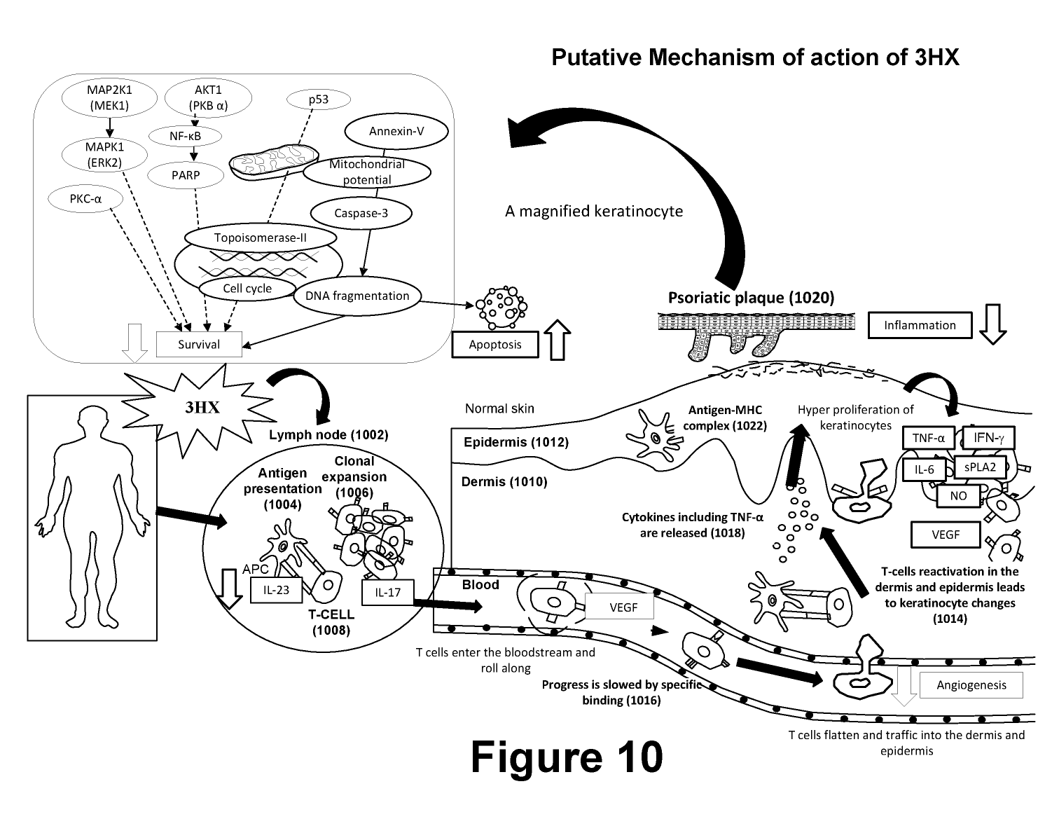

FIG. 10 schematically illustrates certain putative mechanisms of action of medicinal compositions including combinations of Da Huang, Sheng Di Huang and Jin Yin Hua, in accordance with certain embodiments. FIG. 10 further illustrates 3HX targeting of lymph nodes 1002, antigen presentation 1004, clonal expansion 1006, T-cells 1008 that may reactivate in the dermis 1010 and/or epidermis 1012 leading to keratinocyte changes 1014, whereby such progress may be slowed by specific binding 1016, cytokine release 1018, psoriatic plaque 1020, and/or antigen WIC complexes 1022.

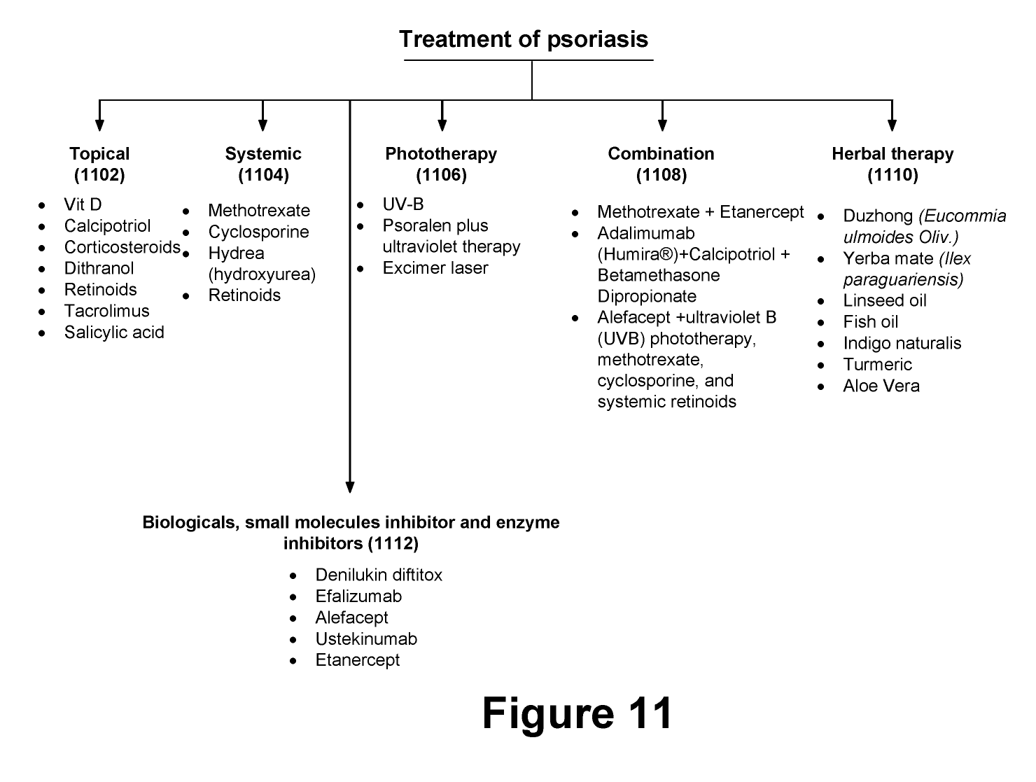

FIG. 11 schematically illustrates certain current psoriasis therapies that may be combined with administration of medicinal compositions including combinations of Da Huang, Sheng Di Huang and Jin Yin Hua, in accordance with certain embodiments. Such therapies may apply to skin, scalp and/or hair disorders, or other inflammatory, autoimmune and/or ontological disorders, that a patient may suffer from other than or in addition to psoriasis. FIG. 11 illustrates that therapies may include one or more topical therapies 1102 such as vitamin D, calcipotriol, corticosteroids, dithranol, retinoids, tacrolimus and/or salicylic acid, one or more systemic therapies 1104 such as methotrexate, cyclosporine, hydrea (hydroxyurea) and/or retinoids, one or more phototherapies 1106 such as UV-B, psoralen plus UV therapy and/or excimer laser therapy, one or more combination therapies 1108 such as methotrexate+etanercept, adalimumab (Humira)+calcipotriol+betamethasone dipropionate, alefacept+UV-B, methotrexate, cyclosporine and/or systemic retinoids, one or more herbal therapies 1110 such as duzhong (eucommia ulmoides oliv.), yerba mate (ilex paraguariensis), linseed oil, fish oil, indigo naturalis, turmeric and/or aloe vera and/or one or more biological inhibitors, small molecule inhibitors and/or enzyme inhibitors 1112 such as denilukin diftitox, efalizumab, alefacept, ustekinumab and/or etanercept.

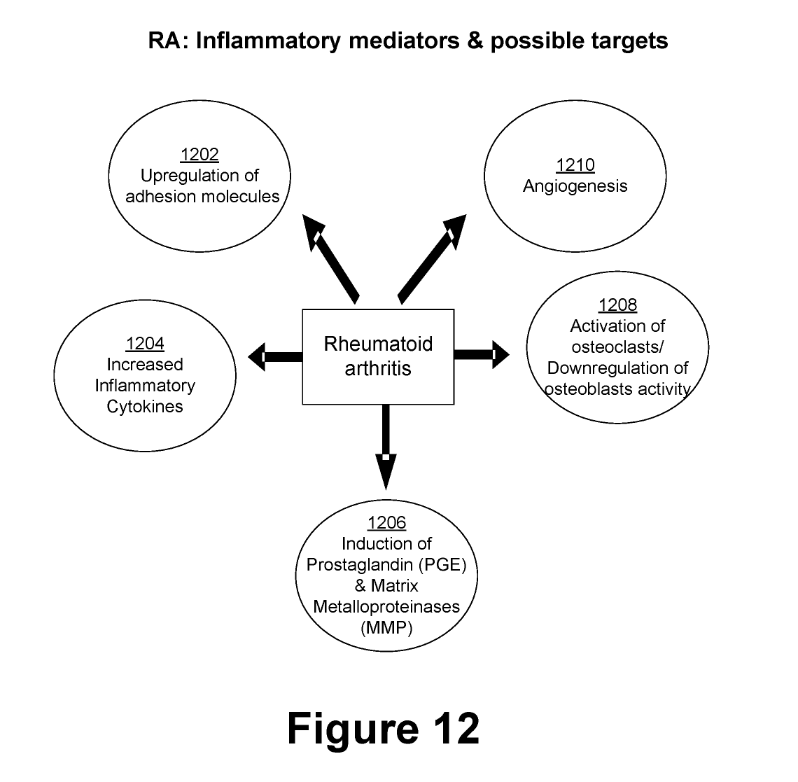

FIG. 12 schematically illustrates certain inflammatory mediators and targets for rheumatoid arthritis (RA) therapies such as medicinal compositions including combinations of Da Huang, Sheng Di Huang and Jin Yin Hua (3HX), in accordance with certain embodiments. FIG. 12 illustrates that administration of 3HX may be provided to a patient to target upregulation of adhesion molecules 1202, increased inflammatory cytokines 1204, induction of prostaglandin (PGE) and matrix metalloproteinases (MMP) 1206, activation of osteoclasts and/or downregulation of osteoblast activity 1208 and/or angiogenesis 1210.

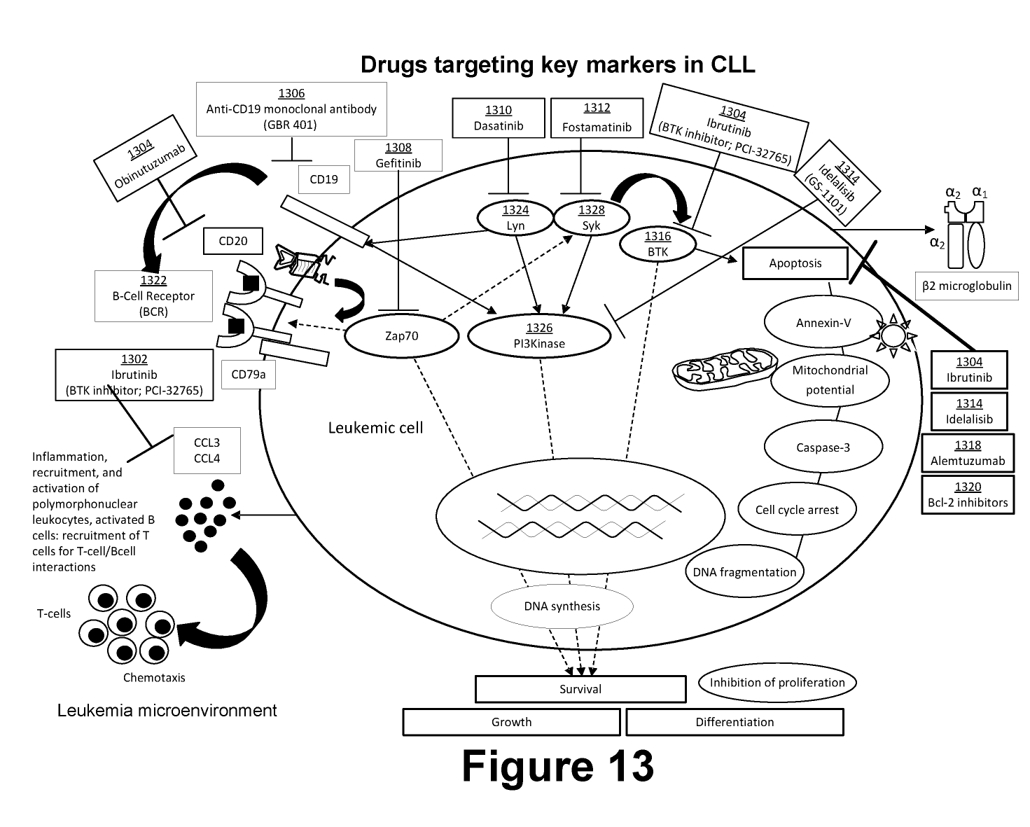

FIG. 13 schematically illustrates certain drugs targeting markers for chronic lymphocytic leukemia (CLL) that may be combined with medicinal compositions including combinations of Da Huang, Sheng Di Huang and Jin Yin Hua (3HX), in accordance with certain embodiments. FIG. 13 illustrates markers targets for treatments for CLL including ibrutinib 1302 or a BTK inhibitor 1316 or PCI-32765 1302, obinutuzumab 1304 or a B-cell receptor inhibitor 1322, anti-CD-19 monoclonal antibody (GBR401) 1306, as well as anti-CD20, anti-CD79a, anti-CCL3, and anti-CCL4, Gefitinib 1308 or a zap70 inhibitor 1308, dasatinib 1310 or a lyn inhibitor 1324 and/or P13kinase inhibitor 1326, fostamatinib 1312 or a syk inhibitor 1328, idelalisib (GS-1101) 1314, alemtuzumab 1318 and Bcl-2 inhibitors 1320. 3HX administration to a patient may be useful in targeting one or more of these mechanisms alone or in combination with a known therapy. Other activities such as may involve beta2 microglobulin may also be influenced by 3HX treatment.

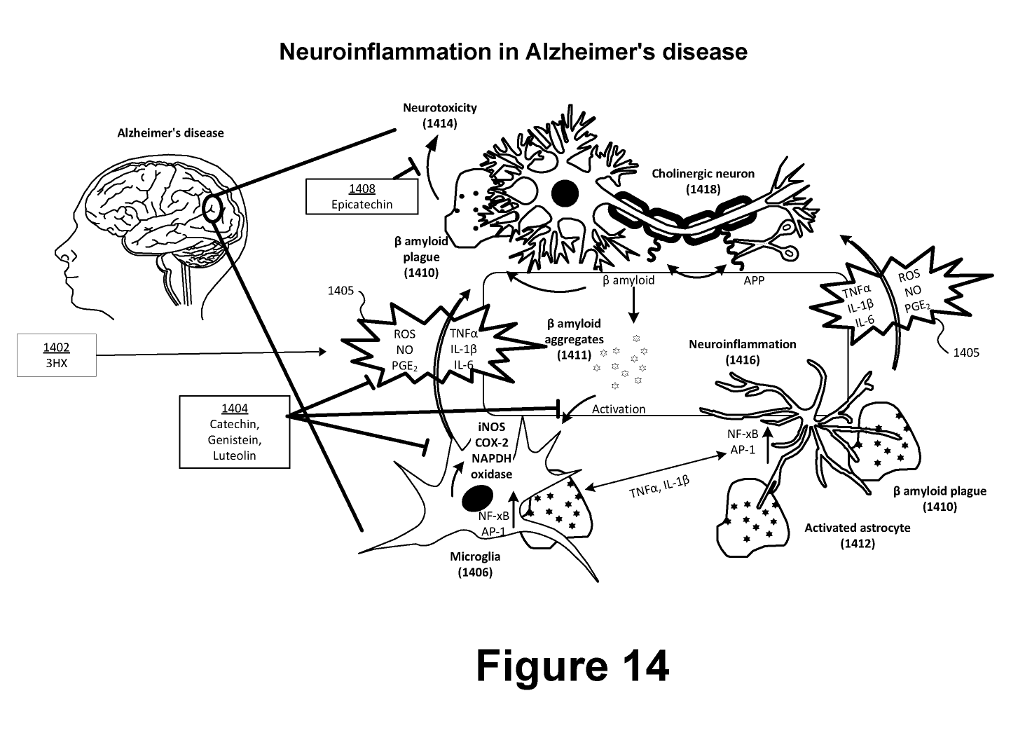

FIG. 14 schematically illustrates neuroinflammation in Alzheimer's disease and certain targets for medicinal compositions including combinations of Da Huang, Sheng Di Huang and Jin Yin Hua (3HX), in accordance with certain embodiments. 3HX 1402 and/or catechin, genistein and/or luteolin 1404 may be administered to target ROS, NO, PGE2, TNF alpha, IL-1beta and/or IL-6 activity 1405 and/or NF-kappaB, AP-1, NOS, COX-2, NAPDH and/or oxidase in microglial cells 1406. Epicatechin 1408 may target beta amyloid plaques 1410, beta amyloid aggregates 1411 and/or activated astrocytes 1412 and/or other factors that can tend to cause or facilitate neurotoxicity 1414 and/or neuroinflammation 1416 and/or affecting cholinergic neuron activity 1418.

### EXAMPLES

Certain embodiments are directed to advantageous medicines and methods of treatment and preparation of medicines and treatments for psoriasis, eczema, melanoma or other skin ailments, inflammation, autoimmune disease, leukemia or another form of cancer wherein combinations of methotrexate and/or another known or discovered treatment with an herbal combination such as Da Huang and Sheng Di Huang, or Da Huang, Sheng Di Huang and Jin Yin Hua, and/or one or more of Mu Dan Pi, Di Gu Pi, Xian He Cao and/or Chun Gen Pi, and/or any one or more of the following eleven additional herbs including Zi Cao, or radix arnebiae (arnebia root) or radix lithospermi (gromwell root), Xuan Shen, or radix scrophulariae (figwort root), Shi Gao or gypsum fibrosum (gypsum), Bai Shao, or radix paeoniae alba (white peony root), Chi Shao or radix paeoniae rubra (red peony root), Hong Hua or flos carthami (safflower), Da Qing Ye or folium isatidis (woad leaf), Qing Dai or indigo naturalis (natural indigo), Bai Zhu or rhizoma atractylodis macrocephalae (largehead atractylodes rhizome), Shi Wei or folium pyrrosiae (shearer's pyrrosia leaf), and/or Rou Gui or cortex cinnamomi (cinnamomum bark).

Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof. or another known treatment described herein or understood or discovered by those skilled in the art may be combined with one, two or all three of Da Huang, Sheng Di Huang and Jin Yin Hua, alone or in combination with certain herbal, molecular or synthetic medicinal dosage compositions, or ingredients thereof, such as digoxin “D” and/or emodin “E,” and/or one or more others described above including Emodin, Rhein, and/or Rhapontin of Da Huang, Carvacrol, Vanillic acid, and/or Sitosterol of Jin Yin Hua, and/or Aucubin, Digoxin, and/or beta-sitosterol of Sheng Di Huang and/or other ingredients present within the two, the three, the seven and/or even the eighteen herbs, are used to inhibit tumor cell growth and/or reduce white cell count.

A known treatment, such as those recited above or elsewhere herein, or a discovered treatment, may be combined with an herbal combination of one or more of the herbs described herein and/or one or more molecules, molecular extracts or herbal ingredients described herein. The known or discovered treatment may be administered before, during and/or after administration of the herbal and/or molecular treatment, and these may be administered together or separately, at the same time or temporally spaced apart in accordance with various embodiments.

In certain embodiments, herbal combinations may include treatment regimens of various per dose amounts and various numbers of doses over the course of the regimen at various dose frequencies depending on the various specific patient-related factors. For example, a regimen of one or more doses may include, in one example, between 13.3 grams to 120 grams of Sheng Di Huang, or 3.3 grams to 60 grams of Da Huang, or both, alone or together, or in combination with Jin Yin Hua and/or one of more other herbs, and/or in combination with additional emodin or digoxin or another molecule described herein, or combinations thereof. The herbal combinations may be administered in a treatment regimen over a period of time including administration of daily doses or 2-3 times daily doses before meals or up to 10× daily doses or less than daily doses even as little as three doses a month, and the herbal combinations may be administered before, during and/or after administration of a regimen of Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof, and/or another known or discovered treatment. Many other example dosage compositions and treatment regimens are provided herein.

Any of Da Huang, Sheng Di Huang and Jin Yin Hua may be administered as a single herb complement or supplement along with another known or discovered treatment regimen. Any two of these herbs, or all three of them, may also be combined and administered as a supplement or complement to another known or discovered treatment regimen. In certain embodiments, the regimen described includes 3.3 grams to 60 grams of Jin Yin Hua. In other embodiments, one or more molecules, molecular extracts and/or molecular compounds are included in the regimen. In further embodiments, one, two, three or all four of the herbs Mu Dan Pi, Xian He Cao, Chun Gen Pi and Di Gu Pi is/are combined with one, two or all of the three herbs Sheng Di Huang, Da Huang and Jin Yin Hua, including 3.3 grams to 15 grams of any of the other four of the seven herbs, e.g., 3.3 grams to 15 grams of Mu Dan Pi, 3.3-10 grams to 15 grams of Xian He Cao, 3.3-10 grams to 15 grams of Chun Gen Pi, and/or 3.3-5 grams to 15 grams of Di Gu Pi.

In an example involving a combination of a known or discovered regimen and a combination of the three herbs, 40 grams of Sheng Di Huang in 100 grams of water, 15 grams of Da Huang in 60 grams of water and 15 grams of Jin Yin Hua in 60 grams of water may be combined in a single or multiple treatment dosage regimen and administered to a patent. The combination may be cooked once or twice, and the herb:liquid ratio may be 1:10 or otherwise, and the herbs may be prepared in multiple ways, including as dry, aqueous or lipidized components. The doses may be administered every 1-10 days or multiple times daily including 2-6 times daily, and even as often as 10 times daily.

In another example, a treatment regimen includes a combination of a known or discovered medicine with one or more of the following seven herbs, such as 5 grams of Mu Dan Pi in 15 grams of water, 20 grams of Sheng Di Huang in 80 grams of water, 5 grams of Xian He Cao in 15 grams of water, 5 grams of Chun Gen Pi in 15 grams of water, 5 grams of Di Gu Pi in 15 grams of water, 10 grams of Da Huang in 40 grams of water and 10 grams of Jin Yin Hua in 40 grams of water. The combination may be cooked once or twice, and the herb:liquid ratio may be 1:10 or otherwise, and the herbs may be prepared in multiple ways, including as dry, aqueous or lipidized components. The dose may be every 1-10 days or multiple times daily including 2-6 times daily, and even as often as 10 times daily.

In another example, a reduced dose formula includes a known or discovered treatment regimen along with lug/ml or more of emodin or 0.05ug/m1 or more of digoxin, or both, and/or 3.3 grams or more of one or more of Da Huang, Sheng Di Huang and Jin Yin Hua, and/or one or more of the following eighteen herbs includes 3.3 grams of Da Huang in about 32 grams water, 13.3 grams of Sheng Di Huang in about 129 grams of water, 3.3 grams of Jin Yin Hua in about 32 grams of water, 3.3 grams of Mu Dan Pi in about 32 grams of water, 5 grams of Di Gu Pi in about 48 grams of water, 10 grams of Xian He Cao in about 97 grams of water, 10 grams of Chun Gen Pi in about 97 grams of water, 8.3 grams of Zi Cao in about 80 grams of water, 6.7 grams of Xuan Shen in about 65 grams of water, 3.3 grams of Shi Gao in about 32 grams of water, 4 grams of Bai Shao in about 39 grams of water, 4 grams of Chi Shao in about 39 grams of water, 3.3 grams of Hong Hua in about 32 grams of water, 8.3 grams of Da Qing Ye in about 80 grams of water, 6.7 grams of Qing Dai in about 65 grams of water, 6.7 grams of Bai Zhu in about 65 grams of water, 5 grams of Shi Wei in about 48 grams of water, and 2 grams of Rou Gui in about 19 grams of water.

In another example, a higher dose formula includes a known or discovered treatment regimen along with 5 ug/ml or more of emodin or 0.1 ug/ml or more of digoxin, or both, and/or 15 grams or more of one or more of Da Huang, Sheng Di Huang and Jin Yin Hua, and/or one or more of the following eighteen herbs: 30 grams of Da Huang in about 300 grams water, 60 grams of Sheng Di Huang in about 600 grams of water, 30 grams of Jin Yin Hua in about 300 grams of water, 15 grams of Mu Dan Pi in about 150 grams of water, 15 grams of Di Gu Pi in about 150 grams of water, 15 grams of Xian He Cao in about 150 grams of water, 15 grams of Chun Gen Pi in about 150 grams of water, 5 grams of Zi Cao in about 50 grams of water, 5 grams of Xuan Shen in about 50 grams of water, 5 grams of Shi Gao in about 50 grams of water, 5 grams of Bai Shao in about 50 grams of water, 5 grams of Chi Shao in about 50 grams of water, 5 grams of Hong Hua in about 50 grams of water, 5 grams of Da Qing Ye in about 50 grams of water, 5 grams of Qing Dai in about 50 grams of water, 5 grams of Bai Zhu in about 50 grams of water, 5 grams of Shi Wei in about 50 grams of water, and 5 grams of Rou Gui in about 50 grams of water.

In another example, a known or discovered treatment regimen is supplemented by a low dose herbal formula including 3.3 grams of two or more of Da Huang, Sheng Di Huang and Jin Yin Hua, and/or one or more of the following seven herbs: 3.3 grams of Da Huang, 13.3 grams of Sheng Di Huang, 3.3 grams of Jin Yin Hua, 3.3 grams of Mu Dan Pi, 5 grams of Di Gu Pi, 10 grams of Xian He Cao, and 10 grams of Chun Gen Pi in water or in a dry or a lipidized formulation or otherwise configured for topical, oral, intravenous or sub-dermal injectable administration.

In another example, a known or discovered treatment regimen is supplemented by a high dose herbal formula including 30 grams or more of Da Huang, and/or with one or more of the following seven herbs: 60 grams of Sheng Di Huang, 30 grams of Jin Yin Hua, 15 grams of Mu Dan Pi, 15 grams of Di Gu Pi, 15 grams of Xian He Cao, and 15 grams of Chun Gen Pi, in water or in a dry or a lipidized formulation or otherwise configured for topical, oral, intravenous or sub-dermal injectable administration.

In another example, a known or discovered treatment regimen is supplemented with a low dose formula including the three herbs including 3.3-20 grams of Da Huang, 10-60 grams of Sheng Di Huang, and 3.3-20 grams of Jin Yin Hua.

In another example, a known or discovered treatment regimen is supplemented with a low dose formula including 3.3-20 grams of Da Huang and 10-60 grams of Sheng Di Huang.

In another example, a known or discovered treatment regimen is supplemented with a low dose formula including 3.3-20 grams of Jin Yin Hua and 10-60 grams of Sheng Di Huang.

In another example, a known or discovered treatment regimen is supplemented with a low dose formula including 3.3-20 grams of Da Huang and 3.3-20 grams of Jin Yin Hua.

In another example, a known or discovered treatment regimen is supplemented with a low dose formula including the one herb including 3.3-30 grams of Da Huang.

In another example, a known or discovered treatment regimen is supplemented with a low dose formula including the one herb including 3.3-30 grams of Jin Yin Hua.

In another example, a known or discovered treatment regimen is supplemented with a low dose formula including the one herb including 10-90 grams of Sheng Di Huang.In another example, a known or discovered treatment regimen is supplemented with a medium dose formula including the three herbs including 10-100 grams of Da Huang, 25-250 grams of Sheng Di Huang, and 10-100 grams of Jin Yin Hua.

In another example, a known or discovered treatment regimen is supplemented with a medium dose formula including 10-100 grams of Da Huang and 25-250 grams of Sheng Di Huang.

In another example, a known or discovered treatment regimen is supplemented with a medium dose formula including 10-100 grams of Jin Yin Hua and 25-250 grams of Sheng Di Huang.

In another example, a known or discovered treatment regimen is supplemented with a medium dose formula including 10-100 grams of Da Huang and 10-100 grams of Jin Yin Hua.

In another example, a known or discovered treatment regimen is supplemented with a medium dose formula including the one herb including 10-100 grams of Da Huang.

In another example, a known or discovered treatment regimen is supplemented with a medium dose formula including the one herb including 10-100 grams of Jin Yin Hua.

In another example, a known or discovered treatment regimen is supplemented with a medium dose formula including the one herb including 25-250 grams of Sheng Di Huang.

In another example, a known or discovered treatment regimen is supplemented with a high dose formula that includes 30-300 grams of Da Huang, 60-600 grams of Sheng Di Huang, and 30-300 grams of Jin Yin Hua.

In another example, a known or discovered treatment regimen is supplemented with a high dose formula that includes 30-300 grams of Da Huang, and 60-600 grams of Sheng Di Huang.

In another example, a known or discovered treatment regimen is supplemented with a high dose formula that includes 30-300 grams of Da Huang and 30-300 grams of Jin Yin Hua.

In another example, a known or discovered treatment regimen is supplemented with a high dose formula that includes 60-600 grams of Sheng Di Huang and 30-300 grams of Jin Yin Hua.

In another example, a known or discovered treatment regimen is supplemented with a high dose formula that includes 30-300 grams of Da Huang.

In another example, a known or discovered treatment regimen is supplemented with a high dose formula that includes 30-300 grams of Jin Yin Hua.

In another example, a known or discovered treatment regimen is supplemented with a high dose formula that includes 60-600 grams of Sheng Di Huang.

In another example, a known or discovered treatment regiment is supplemented with a medium dose herbal formula that includes 10 grams of Da Huang in about 100 grams water, or in dry or lipidized form for oral, topical, sub-dermal or IV administration.

In another example, a known or discovered treatment regiment is supplemented with a medium dose herbal formula that includes 25 grams of Sheng Di Huang in about 250 grams of water, or in dry or lipidized form for oral, topical, sub-dermal or IV administration.

In another example, a known or discovered treatment regimen is supplemented with a medium dose herbal formula that includes 10 grams of Jin Yin Hua in about 100 grams of water, or in dry or lipidized form for oral, topical, sub-dermal or IV administration.

In another example, a known or discovered treatment regimen is supplemented with a medium dose herbal formula that includes 20 grams of Da Huang, 40 grams of Sheng Di Huang, and 20 grams of Jin Yin Hua in about 400 grams of water or more, or in dry or lipidized form for oral, topical, sub-dermal or IV administration.

In another example, a known or discovered treatment regimen is supplemented with a medium dose formula that includes 15 grams of Da Huang, 35 grams of Sheng Di Huang, and 15 grams of Jin Yin Hua in about 300 grams of water or more, or in dry or lipidized form for oral, topical, sub-dermal or IV administration.

In another example, a known or discovered treatment regiment is supplemented with an herbal combination that includes 25-75 grams of Da Huang, 45-135 grams of Sheng Di Huang, and 25-75 grams of Jin Yin Hua in about 250-750 grams of water, or in dry or lipidized form for oral, topical, sub-dermal or IV administration.

In another example, a known or discovered treatment regimen is supplemented with a low dose herbal formula that includes 3.3-10 grams of Da Huang, and 13.3-40 grams of Sheng Di Huang in about 100-300 grams of water, or in dry or lipidized form for oral, topical, sub-dermal or IV administration.

In another example, a known or discovered treatment regimen is supplemented with a high dose herbal formula that includes 30-100 grams of Da Huang and 60-200 grams of Sheng Di Huang in about 600-6000 grams of water, or in dry or lipidized form for oral, topical, sub-dermal or IV administration.

In another example, a known or discovered treatment regimen is supplemented with a medium dose herbal formula that includes 10-50 grams of Da Huang and 30-150 grams of Sheng Di Huang in about 400-2000 grams of water, or in dry or lipidized form for oral, topical, sub-dermal or IV administration.

In another example, a known or discovered treatment regimen is supplemented with a medium dose herbal formula that includes 20-50 grams of Da Huang and 40-100 grams of Sheng Di Huang in about 600-5000 grams of water, or in dry or lipidized form for oral, topical, sub-dermal or IV administration.

In another example, a known or discovered treatment regimen is supplemented with a medium dose herbal formula that includes 15-100 grams of Da Huang and 45-300 grams of Sheng Di Huang in about 600-4000 grams of water, or in dry or lipidized form for oral, topical, sub-dermal or IV administration.

In another example, a known or discovered treatment regimen is supplemented with a medium dose herbal formula that includes 25-75 grams of Da Huang in about 300 grams water, and 50-150 grams of Sheng Di Huang in about 500 grams of water, or in dry or lipidized form for oral, topical, sub-dermal or IV administration.

### LOW DOSE FORMULATIONS AND SUPPLEMENTS

In an example involving a combination of a known or discovered regimen and a low dose combination of the three herbs, 0.01-0.4 grams of Sheng Di Huang in 1-6 grams of water, 0.01-0.2 grams of Da Huang in 0.1-6 grams of water and 0.01-0.4 grams of Jin Yin Hua in 0.1-6 grams of water may be combined in a single or multiple treatment dosage regimen and administered to a patent. The combination may be cooked once or twice, and the herb:liquid ratio may be 1:10 or otherwise, and the herbs may be prepared in multiple ways, including as dry, aqueous or lipidized components. The doses may be administered every 1-10 days or multiple times daily including 2-6 times daily, and even as often as 10 times daily.

In another example, a known or discovered treatment regimen is supplemented with a low dose formula including the three herbs including 0.3-3.3 grams of Da Huang, 1-6 grams of Sheng Di Huang, and 0.3-3.3 grams of Jin Yin Hua.

In another example, a known or discovered treatment regimen is supplemented with a low dose formula including 0.3-3.3 grams of Da Huang and 1-6 grams of Sheng Di Huang.

In another example, a known or discovered treatment regimen is supplemented with a low dose formula including 0.3-3.3 grams of Jin Yin Hua and 1-6 grams of Sheng Di Huang.

In another example, a known or discovered treatment regimen is supplemented with a low dose formula including 0.3-3.3 grams of Da Huang and 0.3-3.3 grams of Jin Yin Hua.

In another example, a known or discovered treatment regimen is supplemented with a low dose formula including the one herb including 0.3-3.3 grams of Da Huang.

In another example, a known or discovered treatment regimen is supplemented with a low dose formula including the one herb including 0.3-3.3 grams of Jin Yin Hua.

In another example, a known or discovered treatment regimen is supplemented with a low dose formula including the one herb including 0.1-10 grams of Sheng Di Huang.

In another example, a known or discovered treatment regimen is supplemented with a low dose formula including the three herbs including 0.1-10 grams of Da Huang, 0.25-25 grams of Sheng Di Huang, and 0.10-10 grams of Jin Yin Hua.

In another example, a known or discovered treatment regimen is supplemented with a low dose formula including 0.10-10 grams of Da Huang and 0.25-25 grams of Sheng Di Huang.

In another example, a known or discovered treatment regimen is supplemented with a low dose formula including 0.1-10 grams of Jin Yin Hua and 0.25-25 grams of Sheng Di Huang.

In another example, a known or discovered treatment regimen is supplemented with a low dose formula including 0.1-10 grams of Da Huang and 0.1-10 grams of Jin Yin Hua.

In another example, a known or discovered treatment regimen is supplemented with a low dose formula including the one herb including 0.1-10 grams of Da Huang.

In another example, a known or discovered treatment regimen is supplemented with a low dose formula including the one herb including 0.1-10 grams of Jin Yin Hua.

In another example, a known or discovered treatment regimen is supplemented with a low dose formula including the one herb including 0.25-25 grams of Sheng Di Huang.

In another example, a known or discovered treatment regimen is supplemented with a low dose formula that includes 0.3-3 grams of Da Huang, 0.6-6 grams of Sheng Di Huang, and 0.3-3 grams of Jin Yin Hua.

In another example, a known or discovered treatment regimen is supplemented with a low dose formula that includes 0.3-3 grams of Da Huang, and 0.6-6 grams of Sheng Di Huang.

In another example, a known or discovered treatment regimen is supplemented with a low dose formula that includes 0.3-3 grams of Da Huang and 0.3-3 grams of Jin Yin Hua.

In another example, a known or discovered treatment regimen is supplemented with a low dose formula that includes 0.6-6 grams of Sheng Di Huang and 0.3-3 grams of Jin Yin Hua.

In another example, a known or discovered treatment regimen is supplemented with a low dose formula that includes 0.3-3 grams of Da Huang.

In another example, a known or discovered treatment regimen is supplemented with a low dose formula that includes 0.3-3 grams of Jin Yin Hua.

In another example, a known or discovered treatment regimen is supplemented with a low dose formula that includes 0.6-6 grams of Sheng Di Huang.

In another example, a known or discovered treatment regiment is supplemented with a low dose herbal formula that includes 0.1-10 grams of Da Huang in about 1-100 grams water, or in dry or lipidized form for oral, topical, sub-dermal or IV administration.

In another example, a known or discovered treatment regiment is supplemented with a low dose herbal formula that includes 0.25-25 grams of Sheng Di Huang in about 2.5-250 grams of water, or in dry or lipidized form for oral, topical, sub-dermal or IV administration.

In another example, a known or discovered treatment regimen is supplemented with a low dose herbal formula that includes 0.1 -10 grams of Jin Yin Hua in about 1.0-100 grams of water, or in dry or lipidized form for oral, topical, sub-dermal or IV administration.

In another example, a known or discovered treatment regimen is supplemented with a low dose herbal formula that includes 0.2-2.0 grams of Da Huang, 0.4-4.0 grams of Sheng Di Huang, and 0.2-2.0 grams of Jin Yin Hua in about 4-40 grams of water or more, or in dry or lipidized form for oral, topical, sub-dermal or IV administration.

In another example, a known or discovered treatment regimen is supplemented with a low dose formula that includes 0.15-1.5 grams of Da Huang, 0.35-3.5 grams of Sheng Di Huang, and 0.15-1.5 grams of Jin Yin Hua in about 3-30 grams of water or more, or in dry or lipidized form for oral, topical, sub-dermal or IV administration.

In another example, a known or discovered treatment regiment is supplemented with an herbal combination that includes 0.25-0.75 grams of Da Huang, 0.45-1.35 grams of Sheng Di Huang, and 0.25-0.75 grams of Jin Yin Hua in about 25-75 grams of water, or in dry or lipidized form for oral, topical, sub-dermal or IV administration.

In another example, a known or discovered treatment regimen is supplemented with a low dose herbal formula that includes 0.33-1.0 grams of Da Huang, and 1.33-4 grams of Sheng Di Huang in about 10-30 grams of water, or in dry or lipidized form for oral, topical, sub-dermal or IV administration.

In another example, a known or discovered treatment regimen is supplemented with a high dose herbal formula that includes 0.30-1.0 grams of Da Huang and 0.6-2.0 grams of Sheng Di Huang in about 6-60 grams of water, or in dry or lipidized form for oral, topical, sub-dermal or IV administration.

In another example, a known or discovered treatment regimen is supplemented with a low dose herbal formula that includes 0.10-0.50 grams of Da Huang and 0.30-1.5 grams of

Sheng Di Huang in about 4-20 grams of water, or in dry or lipidized form for oral, topical, sub-dermal or IV administration.

In another example, a known or discovered treatment regimen is supplemented with a low dose herbal formula that includes 0.20-0.50 grams of Da Huang and 0.40-1.0 grams of Sheng Di Huang in about 6-50 grams of water, or in dry or lipidized form for oral, topical, sub-dermal or IV administration.

In another example, a known or discovered treatment regimen is supplemented with a low dose herbal formula that includes 0.15-1.0 grams of Da Huang and 0.45-3.0 grams of Sheng Di Huang in about 6-40 grams of water, or in dry or lipidized form for oral, topical, sub-dermal or IV administration.

In another example, a known or discovered treatment regimen is supplemented with a low dose herbal formula that includes 0.25-0.75 grams of Da Huang in about 1-3 grams water, and 0.10-1.50 grams of Sheng Di Huang in about 1-5 grams of water, or in dry or lipidized form for oral, topical, sub-dermal or IV administration.

Examples of medium and high dose, and reduced and low dose herbal formulas for supplementing a known or discovered treatment regimen before, during or after administration of said known or discovered treatment regimen include one or more of the other herbs described herein, such as any of herbs contained in the seven herb and eighteen herb combinations described herein, that are administered in doses that are the same as or similar to or commensurate with the medium and high dose, and reduced and low dose example formulas for the one, two and three herb combinations described herein, including corresponding percentage changes when four or more herbs are included in the supplemental herbal formula that is administered before during or after a known or discovered treatment regimen. Many other combinations may be used depending on characteristics of the patient such as age and weight, the condition of the patient, and the patient's history.

Doses in between the low dose and high dose examples for the formulas that include one, two, or three or more herbs, or the seven herb formula, or the eighteen herb formula are also within the scope of further examples with per herb doses and/or total herb doses that are within the ranges provided in the examples above. Under certain conditions, doses above the high dose formula or below the low dose formula may be used as determined by a physician using his or her expertise and experience both generally in the field and with specific patients. In addition, other combinations of two, three, four, five, or more of these 18 herbs, and/or including one or more other herbs as understood by those skilled in the art, may be used in further examples of formulas wherein the dose ranges described in the above examples or otherwise as determined by a physician may be used. Further herbs not described herein may also be included in formulas including combinations of the described herbs and/or molecules, molecular extracts or molecular compounds described herein or understood by those skilled in the art.

Surprise effectiveness is also provided by very low dose herbal combinations of Da Huang, Sheng Di Huang and Jin Yin Hua and by combinations of low doses of one or more of Da Huang, Sheng Di Huang and JinYin Hua with certain herbal extracts, molecules and/or known medicines.

In the examples above and below herein, certain doses of herb combinations have been described in certain amounts of water. Mixing the herbs in water is only one example of a way to take the herbal combinations. Instead, no water may be used and instead another liquid such as DMSO or an oil may be used. The herbs may be formed into pills or capsules, or a syringe or an IV may be used. Any of the herbal combinations may be formed into a cream and rubbed onto the skin or hair, or a syringe may be used to inject a patient with an herbal combination.

In another example, the effect of CXCR4 antagonists on the survival of NB4, HL60, Jurkat leukemic cells and HT29 colon cancer and prostate tumor cells was examined. Digoxin significantly inhibited the growth of leukemic cells at concentrations between (0.05-to 1 microgram/ml). Emodin by itself inhibited the growth of leukemic cells only at concentrations of more than 5 microgrm/ml. Combinations of digoxin at concentrations of 0.1 microgram/ml and either 5 or 10 microgram/ml of emodin increased significantly the tumor killing ability of both compounds. Digoxin, Emodin, and their combination also add partial however significant effect on HT29 tumor cells.

Certain treatments may be prepared as a mixture of herbs that are known to have separately certain molecular components or medicinal advantages. For example, emodin may be extracted from traditional medicinal plants such as Rhei Rhizoma and Rheum Palmatum. In one embodiment, the source of Emodin used is Da Huang-Chinese name, or Rhubarb Root-English name, or Rheum Plamatum-Botanical name, or Radix Rhisoma Rhei-Pharmaceutical name. Emodin may be extracted from Rhubarb, Buckthorn and/or Japanese Knotweed (Fallopia Japonica). Aloe-emodin may be used which is a variety of emodin found in Socotrine, Barbados, and Zanzibar aloes.

A medicinal treatment is prepared in certain embodiments by mixing the herb, Da Huang, in water at a ratio of approximately 10:1. The herb may be ground to a fine powder. The water may be added to the fine powdered herb, and the pot covered. After boiling, the heat is lowered in certain embodiments to about 70 degrees centigrade. The aqueous mixture is cooked for another hour. The liquid is then strained into a container. In some cases, this may be done twice. In the second cooking, the ratio may be reduced to 7.5:1. The second cooking may take about 45 minutes including the boiling. Other herbs including Sheng Di Huang and/or Jin Yin Hua may be mixed with the Da Huang or prepared alone or in combination with other herbs. Dry or lipidized capsules, or a cream, or a topical injection solution, or an IV solution, or other formulations understood to those skilled in the art may also be provided for administration of the treatment. The methotrexate or other known or described or discovered treatment that is administered before during and/or after the herbal medicinal treatment in accordance with certain embodiments may also be prepared in multiple ways as understood by those skilled in the art.

Single herbs or combinations of two or more herbs alone or with any one or more of the described molecules may be prepared in a process involving the following or a subset or variation thereof: grinding the herbs to a fine texture in the mixer for around 2-3 min until it looked fine powder; weighing the powder (e.g., 25 gm) and transferring to a beaker (e.g., 2000 ml); adding distilled water (RT) to powdered herbs in a ratio of 1:20 (gm of herbs:ml of water) and soaking the herbs for 15-20 min; boiling the mixture to 85-90° C.; cooling the temperature of the mixture down to 70-75° C. after removing it from the hot plate; covering the beaker properly with aluminum paper and cooking the mixture at 70° C. on hot plate-the total time of cooking of the mixture may be approximately 60 min which includes boiling, cooling it down and cooking; straining the mixture with the help of a manual strainer; after filtration, centrifuging the extract at 5000 rpm for 15 min and collecting the supernatant; filter sterilizing the supernatant by passing through 0.2 μm syringe filter; storing the clear filtrate at 4° C-subsequent dilutions may be prepared.

A source of Digoxin in certain embodiments is Sheng Di Huang-Chinese name, or Foxglove root-English name, or Radix Rhemania-Pharmaceutical name. The preparation of the Digoxin may be the same as for the Emodin. In certain embodiments, the herbs from which the Emodin and Digoxin are extracted are cooked together. Digoxin may be extracted from Digitalis Purpurea or Purple Foxglove.

In certain embodiments, a prepared treatment may not be “pure.” For example, certain treatments may involve a “vegetable soup” type regiment made of more than one and even two, three, seven or eighteen herbal ingredients per specific examples provided herein, or any combinations of the herbs described herein, or combinations of the herbs described herein with other herbs not mentioned herein. In certain embodiments, herbal ingredients are mixed in solution and a patient may drink the liquid. While freeze-drying the herb in powder form may be possible, the above-described process appears to be more effective.

Harvested non adherent human hematopoietic cancer cell lines NB4 (AML), HL60 and Jurkat were seeded at 2×10⁵ viable cells/1 ml per well into a 24-well plate in triplicates in a medium supplemented with 10% FCS and incubated with different concentrations of digoxin, Emodin, and their combination for 24 hours. Following the incubation, the cells were stained with propidium iodide (PI) (Sigma, St. Louis, Mo.) and percent of viable PI-negative cells in culture was determined by FACScalibur analysis (Becton Dickinson Immunocytometry Systems), using CellQuest software. Adherent prostate cancer PC3 cells and colon cancer HT29 cells were seeded at 1×10⁵ viable cells/1 ml per well into a 24-well plate under conditions described above, and following 24-hour exposure to digoxin, Emodin, and their combination, the cells were harvested, washed with PBS and stained with PI and counted as described for hematopoietic cells.

Also provided herein are treatments with an advantageous combination of a known or discovered treatment and one or more of Da Huang, Sheng Di Huang and Jin Yin Hua for patients suffering with psoriasis, eczema, melanoma or other skin disease, inflammation, autoimmune disorder, and/or cancer.

A treatment regimen, that includes a known or discovered treatment along with periodic doses of an advantageous herbal combination, is provided to treat psoriasis, eczema, melanoma or other skin disease, inflammation, autoimmune disorder, and/or cancer. The herbal combination may include in certain embodiments Jin Yin Hua, Sheng Di Huang and Da Huang, otherwise referred to herein as the three herb combination or “3HX.”. Another treatment regimen includes a known or discovered treatment and one or more of emodin and digoxin and/or another of the molecules described herein, and/or one or more of Jin Yin Hua, Sheng Di Huang and Da Huang, and/or one or more of Mu Dan Pi, Di Gu Pi, Xian He Cao and Chun Gen Pi. The treatment regimen may be administered topically, orally, subcutaneously and/or intravenously, and the known of discovered treatment may be administered before, during and/or after administration of the herbal combination.

### Nanogel Formulation and Method of Preparing a Nanogel

A nanogel, nanochrystals and/or another nano-particulate formulation is provided in accordance with certain embodiments, as are methods of preparing such nano-particulate formulation and methods of treating certain conditions with an herbal nano-particulate combination including da huang, sheng di huang or jin yin hua, or combinations thereof, alone or in combination with one or more known or discovered medicines, e.g., one or more of the known medicines for treatment one or more of the conditions described herein.

A nano-particulate preparation process may include grinding and/or homogenizing to reduce the particle size of the herbal mixture to approximately 500 nm average particle size or lower. In certain embodiments, grinding and/or homogenizing may include Dyno milling, e.g., run once to reduce the herbal mixture particulate size to an average 250 nm particle size or lower, or run multiple times to reduce the herbal mixture particulate size to approximately an average 150 nm particle size or lower.

A high pressure homogenizer may be used. The mixture may be pushed through a filter, e.g., 0.2 μm or 0.1-0.3 μm or 0.1-0.4 μm or 0.15-0.3 μm or 0.15-0.4 μm).

The process may include sonication. A step in the process of formulating a nano-particulate mixture may include utilizing ultrasound.

A cooking process may be performed before or after the nano-particulate mixture is prepared that includes Da Huang, Sheng di Huang or Jin Yin Hua, or combinations thereof, in accordance with certain embodiments.

A straining process may be performed to get an aqueous extract. Note that there is a limit on the volume of extract that can be tolerated by a human patient for administration of medicine and/or a medicinal supplement.

A process of preparing a nano-particulate mixture may include lyophilization or freeze drying. For preparation of in vitro formulations, DMSO may be used, but not for in vivo formulations because of the toxicity of DMSO in animals and humans.

A process of preparing a nano-particulate mixture in certain embodiments may include solubilization and/or selection of a concentration of extract. The process may include formulation of a cream, lotion, gel, shampoo, tablet, capsule, nano-lipid carrier, nanogel, nano-chystals, nano-particulates, patch, IV or subdermal formulation or other formulation described herein or as understood by those skilled in the art for administering medicine and/or medicinal supplements to a patient.

### EXAMPLE NANOGEL/NANO-PARTICULATE TOPICAL FORMULATION

An example nanogel formulation may include 1-20%, or or 1-15%, 1-10%, or 2.5-10% or 2.5-5% or approximately 2.5% or approximately 5% of an herbal mixture that includes Da Huang, Sheng Di Huang or Jin Yin Hua or combinations thereof. An example nanogel formulation in accordance with certain embodiments may include fulvic acid, e.g., up to 1-10% or 2.5-10% or 2.5-5% or 1-5% or 5-10%.

An example nanogel or nano-particulate lotion, cream, ointment or shampoo formulation may include carbopol ultraze 21/polymer surfactant. Triethanolomine may be advantageously used to convert lotion to gel and/or to neutralize a level of ph to between 5-8, 5.5-7.5 or 6-7 for topical application.

Propylene glycol and/or polyethylene glycol may be included in a nanogel or other nano-particulate topical formulation, e.g., each 0%-15% each or 5%-10% each, or up to 15% total, or up to 20% total or up to a concentration level wherein spreadability may begin to become too low depending on other ingredients and topical administration considerations.

An example nanogel or other nano-particulate topical formulation may include DMDM Hydantoin.

A nano-liquid carrier may be included in a nano-particulate topical formulation in accordance with certain embodiments, e.g., stearic acid. The nano-particulate topical formulation may be lyophilized and homogenized with a dyno-mill, not exceeding a lipid limit, in an example embodiment.

Certain embodiments specifically do not include any parabins nor benzoid, as these have been deemed capable of effecting long term toxicity issues in certain patients or in a certain percentage of patients.

Hydrophyllic capacity is advantageously taken into account in certain embodiments such that permeability is greatly enhanced in a nanogel or other nano-particulate topical formulation including less than 450 nm particulates.

An example nano-particulate tablet formulation may include piperine, e.g., 1-10% or 2.5-10%, or 5-10% or 1-5% or 2.5-5%. A tablet formulation may be hardened above 40° C-50° C. A nano-particulate tablet formulation may be reduced in size for ease of oral administration due to the enhanced permeability of nano-particulates compared with larger particulate sizes, e.g., above 450 nm. The permeability may be increased, e.g., from 10-20% to 20-80% or 30-70% or 40-60% or 50-60% and nano-particulates in accordance with certain embodiments advantageously may be packed stably into tablets of smaller sizes.

A nanogel or other nano-particulate topical formulation may include 1-20% or 1-15% or 1-10% or 2.5-10% or 2.5%-5% of the nano-particulate mixture. Nano-particulates may be 150-450 nm or 150-350 nm or 150-250 nm, or 100-300 nm or 100-400 nm or 100-450 nm or 50-450 nm, which serve to administer more of the medicine or medicinal supplement to a patient than the 10-20% permeability or lower of example formulations having 450 nm and above particulate sizes, because of reduced cumulative dose toxicities, better stability, and enhancement of treatment for the patient regarding tolerance and saturation issues.

A nano-emulsion, which tends to be oily, may include 100 nm or less particulate sizes.

Treatment methods and medicinal compositions are provided for treating eczema, and other skin conditions. Certain embodiments involve the use of herbal combinations and combinations of certain herbs, certain herbal extracts and/or certain molecular components of certain herbs, alone or in combination with or supplemental to one or more other known or novel or experimental treatments. Certain embodiments are formulated as a shampoo, conditioner, cream, ointment or other topical scalp or hair treatment.

Methods and medicinal compositions are provided to treat eczema, and/or other skin conditions such as psoriasis, psoriatic arthritis, or other inflammatory skin disorders, dandruff including seborreic dandruff, seborrhea, acne, burns, dermatitis including atopic dermatitis, warts, keratosis, acne, alopecia, hirsutism or capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, capuche sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or warts or burns, or another skin ailment, lesion or sore, particularly of the scalp and also for other skin regions affected by an ailment that is susceptible to topical or hair treatment, as well as hair fall or hair loss conditions.

In certain embodiments, herbs are advantageously combined as herbal combinations including Da Huang and Sheng Di Huang. Certain embodiments also include Jin Yin Hua. Other embodiments include one or more of Mu Dan Pi, Di Gu Pi, Xian He Cao, and Chun Gen Pi. Certain embodiments include herbal extracts or molecular components such as emodin, digoxin, and/or other molecules such as aucubin, beta-sitosterol, vanillic acid, rhein, rhapontin and carvacrol. Combinations of these and other herbs, herbal extracts and/or molecules described herein are provided in various embodiments.

Along with certain combinations of herbs and/or herbal extracts, emotives or molecular components as described herein, a shampoo, conditioner, cream, ointment or other topical eczema treatment in accordance with certain embodiments may include one or more other components including one or more surfactants and/or co-surfactants, thickening agents, pH adjusters, buffers, aesthetic additives, water, hydro-alcoholic hair serum and/or another dispersive agent, solvent, solubilizer or vehicle, hair-fall or hair-loss control actives, conditioners, preservatives and/or moisturizers and/or vitamins, humectants, one or more cationic polymers, silicone, a suspending agent, perfume and/or other additives that may be consistent with a healthy shampoo, conditioner, cream, ointment or other topical eczema treatment.

FIG. 7 illustrates the components of a shampoo in accordance with certain embodiments. Referring to FIG. 7, an example of a shampoo in accordance with certain embodiments is illustrated as including nine generalized components. These nine components include a surfactant, a thickening agent, a pH adjuster/buffer, an aesthetic additive, water, a conditioner, one or more active herbs or herbal extracts or molecules, a preservative and moisturizers/vitamins. Shampoos in accordance with various alternative formulations may include fewer than all of these nine components and they may include other active or inactive components known to those skilled in the art as having some advantage when included in a shampoo formula or in a medicinal combination for treating eczema, or another skin condition such as psoriasis, melanoma, or dermatitis or hair loss or another head or scalp disorder or ailment.

A shampoo in accordance with certain embodiments includes one or more surfactants that may be known or discovered as being advantageous for cleaning hair with a shampoo. A primary surfactant may be included to provide flash foam for cleaning the hair by removing dirt and other impurities. A secondary surfactant may be included to provide stable foam and to reduce the harshness of the primary surfactant. A surfactant may be used that includes a charged, hydrophilic head group and a long, hydrophobic alkyl chain tail. Surfactants are configured to break molecular bonds between dirt and hair and to transport the dirt into an aqueous medium to be rinsed free from the hair and scalp. Examples of surfactants that may be contained in a shampoo in accordance with certain embodiments include sodium laureth sulphate, ammonium laureth sulfate, and sodium cocoyl isethionate. Examples of co-surfactants include cocamide MEA and cocoamidopropyl betaine.

A shampoo in accordance with certain embodiments may include a thickening or suspending agent. Examples of thickening or suspending agents that may be contained in accordance with certain embodiments include carbomer and PEG 150 distearate. The thickening agent may be included to stabilize the shampoo during storage and/or to prevent the setting or dumping of pigments and silicone.

A pH adjuster or buffer may be included in a shampoo in accordance with certain embodiments. An example of a pH adjuster or buffer includes citric acid, tartaric and sodium hydroxide. The pH adjuster or buffer is configured to cause the shampoo to be gentle to the skin. A lower pH may cause hair to be compact and to shine and to protect the surfactant from hydrolysis, and as such, the pH adjuster or buffer may serve to lower the pH of the shampoo. However, alternative embodiments include pH adjusters that serve to raise the pH of a shampoo that contains an herbal formula that exhibits an exceptionally low pH.

An aesthetic additive may be included in a shampoo in accordance with certain embodiments. Examples of aesthetic additives include colorants, opacifiers, UV absorbers, perfumes and natural and artificial fragrances.

One or more conditioners may be included in a shampoo in accordance with certain embodiments. The one or more conditioners may include a cationic polymer such as guar hydroxypropyl trimonium chloride. The one or more conditions may include silicone and/or a silicone emulsion such as dimethiconol, dimethicone, or amodimethicone. The silicone and/or silicone emulsion may serve to coat the hair and cause the hair to become soft, smooth and shiny.

A shampoo in accordance with certain embodiments includes one or more active herbs or herbal extracts or emotives that are described in several examples herein. These one or more active herbs or herbal extracts serve to promote treatment of eczema, and/or another condition such as psoriasis, dermatitis, melanoma, hair loss and other hair or scalp conditions described herein or understood by those skilled in the art.

A preservative may be included in a shampoo in accordance with certain embodiments. The preservative may be configured to prevent microbial growth. Examples of preservatives that may be contained in a shampoo in accordance with certain embodiments include paraben free, formaldehyde donor free and halogenated free.

A moisturizer and/or one or more vitamins may be included in a shampoo in accordance with certain embodiments. Examples include combinations of D-Panthenol, vitamin E acetate, sodium PCA, glycerine and one or more amino acids. The moisturizer and/or vitamins may be configured to penetrate into hair shaft, seal cuticles and keep hair moisturized.

A sulphate free eczema shampoo may be formulated with the following ingredients:

An active herbal combination of Da Huang, Sheng Di Huang and Jin Yin Hua, aqua (DM water), PEG 80 Sorbitan Laurate, Sodium Lauroyl Sarcosinate, Disodium EDTA, Niacinamide, Citric Acid.

### Sodium Benzoate, and Phenoxy Ethanol

In alternative embodiments, the active herbal combination may include Da Huang and Sheng Di Huang without Jin Yin Hua, or Da Huang and Jin Yin Hua without Sheng Di Huang, or Sheng Di Huang and Jin Yin Hua without Da Huang, or Da Huang alone with Sheng Di Huang nor Jin Yin Hua, or Sheng Di Huang alone without Da Huang nor Jin Yin Hua, or Jin Yin Hua alone without Da Huang nor Sheng Di Huang.

In alternative embodiments, a sulphate free eczema shampoo may include PEG-4 Dilaurate, PEG-7 Glyceryl Cocoate, one or more PEG-20 Almond Glycerides, and/or PEG-25 Hydrogenated Castor Oil instead of or in addition to PEG 80 Sorbitan Laurate.

In alternative embodiments, an eczema shampoo may include sodium lauryl sulphate instead of or in addition to Sodium Lauroyl Sarcosinate, although an eczema shampoo in accordance with these alternative embodiments would not be sulphate free.

In alternative embodiments, a sulphate free eczema shampoo may include ascorbic acid instead of or in addition to citric acid.

The sulphate free eczema shampoo may further include one or more of the following additional components: CAPB; or Glycerine, Diethylene Glycol, Propylene Glycol, and/or one or more Ceramides, Oils and/or Butters; or PEG 4 rapseedamide, Macrogol 200, and/or Polyoxyethylen(4); or Perfume allergen free and/or any GRAS natural perfume; or Poly Quat 10, Poly Quat 4, and/or Poly Quat 44; or PEG 150 Distearate, Antil 120 Plus, and/or Antil 127; or Olive Leaf Extract; Or combinations of two or more of these additional components.

A sulphate free eczema shampoo may be formulated with the following ingredients:

An active herbal combination of Da Huang, Sheng Di Huang and Jin Yin Hua (6mg), aqua (DM water), CAPB, PEG 80 Sorbitan Laurate, Sodium Lauroyl Sarcosinate, Glycerine, PEG 4 rapseedamide, Perfume allergen free, Disodium EDTA, Niacinamide, Poly Quat 10, Citric Acid, PEG 150 Distearate, Sodium Benzoate, Phenoxy Ethanol, and Olive Leaf Extract.

In alternative embodiments, the active herbal combination may include Da Huang and Sheng Di Huang without Jin Yin Hua, or Da Huang and Jin Yin Hua without Sheng Di Huang, or Sheng Di Huang and Jin Yin Hua without Da Huang, or Da Huang alone with Sheng Di Huang nor Jin Yin Hua, or Sheng Di Huang alone without Da Huang nor Jin Yin Hua, or Jin Yin Hua alone without Da Huang nor Sheng Di Huang.

A shampoo may be formulated with the following ingredients:

An active herbal combination of Da Huang, Sheng Di Huang and Jin Yin Hua (6 mg), Aqua (D M Water), Sodium Laureth Sulphate 70%, Cocomonoethanolamide, Dimethiconol (and) Triethanolamine-Dodecylbenzenesulfonate, Ethylene Glycol Distearate, Sodium Chloride, D-Panthenol, Polyacrylic Acid 940, Guar Hydroxypropyltrimonium Chloride, Methylchloroisothiazolinone and Methylisothiazolinone, Sodium Hydroxide, Ethylenediaminetetraacetic acid tetrasodium salt, Tocopheryl acetate and Citric Acid.

In alternative embodiments, an eczema shampoo may include sodium lauryl sulphate instead of or in addition to sodium laureth sulphate 70%.

In alternative embodiments, a shampoo may include SLES, SLS, ALES, and/or ALS instead of or in addition to Cocomonoethanolamide.

In alternative embodiments, a shampoo may include glycol stearate and/or glycol stearate SE instead of or in addition to ethylene glycol distearate.

In alternative embodiments, a shampoo may include Coconut oil and/or avocado oil instead of or in addition to D-Panthenol.

In alternative embodiments, a shampoo may include Polyvinylalcohol (PVA) instead of or in addition to Polyacrylic Acid 940.

In alternative embodiments, a shampoo may include Potassium hydroxide instead of or in addition to Sodium Hydroxide.

In alternative embodiments, a shampoo may include ascorbic acid instead of or in addition to citric acid.

A shampoo may also include perfume, perfume baby splash and/or any GRAS natural perfume.

A shampoo may also include Cocoamidopropylbetaine instead of or in addition to Cocomonoethanolamide.

A hair lotion may be formulated as follows:

DM Water, an active herbal combination of Da Huang, Sheng Di Huang and Jin Yin Hua (6 mg), Propylene Glycol, Glyceryl Monostearate SE, Light Liquid Paraffin, Cetyl alcohol, Stearic acid, Cyclopentasiloxane, Phenoxyethanol &Triethylene glycol, Triethanolamine, Ethylenediaminetetraacetic acid tetrasodium salt and Alkyl Acrylates.

In alternative embodiments, an eczema shampoo may include 1,3-Propanediol instead of or in addition to Propylene Glycol.

In alternative embodiments, a hair lotion may include white soft paraffin instead of or in addition to Light Liquid Paraffin.

In alternative embodiments, a hair lotion may include cationic guar, one or more silicones, Honeyquat, and/or one or more polyquats instead of or in addition to Cetyl alcohol.

In alternative embodiments, a hair lotion may include Emulsifying wax, Beeswax instead of or in addition to stearic acid.

In alternative embodiments, a hair lotion may include C13-16 isoparaffin (and) C12-C14 isoparaffin (and) C13-C15 alkane instead of or in addition to Cyclopentasiloxane.

In alternative embodiments, a hair lotion may include Biotin, methyparaben, carbomer, cetyl alcohol, and/or propylparaben instead of or in addition to Triethanolamine.

A hair lotion for treating eczema may further include Coconut oil, olive oil, butter, bacon grease, peanut oil and/or or macadamia oil.

A hair lotion for treating eczema may also include perfume, perfume baby splash and/or any GRAS natural perfume.

A method of treating a skin or scalp condition in accordance with certain embodiments may include applying to the skin or scalp a medicinal composition formulated as a shampoo, conditioner, cream, ointment or other topical scalp or hair treatment that comprises a low concentration formula 5-10 grams or more of an active herbal combination of Sheng Di Huang, Da Huang and Jin Yin Hua in an example 16 fluid ounce formulation. A high concentration formula may include 50-150 grams of the active herbal combination in an example 16 fluid ounce formulation. A medium concentration formula may include 10-50 grams of the active herbal combination in an example 16 fluid ounce formulation.

In certain embodiments, the medicinal composition may include 1%-20% of the active herbal combination. In certain embodiments, the medicinal composition may include 2.5%-10% of the active herbal combination. In certain embodiments, the medicinal composition may include 2.5%-5.0% of the active herbal combination. In certain embodiments, the medicinal composition may include 3.5 wt. % or more of Da Huang. In certain embodiments, the medicinal composition may include 15.4 wt. % or more of a combination of Da Huang and Sheng Di Huang.

In certain embodiments, the herbal combination may include 2-20 grams of Jin Yin Hua, 2-20 grams of Da Huang and 6-60 grams of Sheng Di Huang. In certain embodiments, the medicinal composition may include 0.2 wt. %-4 wt. % of Jin Yin Hua, 0.2 wt. %-4 wt. % of Da Huang, and 0.6 wt. %-12 wt. % of Sheng Di Huang.

In certain embodiments, the herbal combination may include13.3-60 grams of Sheng Di Huang, 3.3-15 grams of Da Huang, and 3.3-15 grams of Jin Yin Hua.

In certain embodiments, the combination of Sheng Di Huang, Da Huang and Jin Yin Hua comprises 20 wt. %-80 wt. % of the active herbal combination. For example, the active herbal combination may include 12 wt. %-48 wt. % of Sheng di Huang, 4 wt. %-16 wt. % of Da Huang and 4 wt. %-16 wt. % of Jin Yin Hua.

The active herbal combination may include 18 wt. % or more of Sheng Di Huang, 6 wt. % or more of Da Huang and 6 wt. % or more of Jin Yin Hua.

A medicinal composition is also provided, in accordance with certain embodiments, that may be formulated as a shampoo, conditioner, cream, ointment or other topical skin, scalp or hair treatment, comprising an herbal combination of 13.3 grams or more of Sheng Di Huang and 3.3 grams or more of Da Huang in a 16 fluid ounce formulation. In certain embodiments, the herbal combination also includes 3.3 grams or more of Jin Yin Hua.

The medicinal composition may include 0.5 wt. % or more of Da Huang and 1.5 wt. % or more of Sheng Di Huang. In certain embodiments, the medicinal composition also includes 0.5 wt. % or more of Jin Yin Hua.

The medicinal composition may include 0.5 wt. %-1.5 wt. % of Da Huang and 1.5 wt. %-4.5 wt. % of Sheng Di Huang. In certain embodiments, the medicinal composition also includes 0.5 wt. %-1.5 wt. % of Jin Yin Hua.

The medicinal composition may include 6 wt. % or less of Da Huang and 18 wt. % or less of Sheng Di Huang. In certain embodiments, the medicinal composition includes 6 wt. % or less Jin Yin Hua.

The active herbal combination may include 40 wt. % or more of Sheng Di Huang and 10 wt. % or more of Da Huang. In certain embodiments, the active herbal combination may include 10 wt. % or more Jin Yin Hua.

The active herbal combination within the medicinal composition may include 22.4 wt. % or more, 30 wt. % or more or 60 wt. % or more of the combination of Sheng Di Huang, Da Huang and Jin Yin Hua.

The active herbal combination may include 15.4 wt. % or more of Sheng Di Huang, 3.5 wt. % or more of Da Huang and 3.5 wt. % or more of Jin Yin Hua.

The active herbal combination may include 18 wt. % or more of Sheng Di Huang, 6 wt. % or more of Da Huang and 6 wt. % or more of Jin Yin Hua.

Other medicinally active or inactive components may be included in combination with the herbal combinations described herein. The herbal combination may be administered alone or in combination with or supplemental to one or more other medicinal treatments.

Referring to FIG. 7, an example of a shampoo in accordance with certain embodiments is illustrated as including nine generalized components. These nine components include a surfactant, a thickening agent, a pH adjuster/buffer, an aesthetic additive, water, a conditioner, one or more active herbs or herbal extracts or molecules, a preservative and moisturizers/vitamins. Shampoos in accordance with various alternative formulations may include fewer than all of these nine components and they may include other active or inactive components known to those skilled in the art as having some advantage when included in a shampoo formula or in a medicinal combination for treating eczema, or another skin condition such as psoriasis, melanoma, or dermatitis or hair loss or another head or scalp disorder or ailment.

A shampoo or other topical treatment in accordance with certain embodiments includes one or more surfactants that may be known or discovered as being advantageous for cleaning hair with a shampoo. A primary surfactant may be included to provide flash foam for cleaning the hair by removing dirt and other impurities. A secondary surfactant may be included to provide stable foam and to reduce the harshness of the primary surfactant. A surfactant may be used that includes a charged, hydrophilic head group and a long, hydrophobic alkyl chain tail. Surfactants are configured to break molecular bonds between dirt and hair and to transport the dirt into an aqueous medium to be rinsed free from the hair and scalp. Examples of surfactants that may be contained in a shampoo in accordance with certain embodiments include sodium laureth sulphate, ammonium laureth sulfate, and sodium cocoyl isethionate. Examples of co-surfactants include cocamide MEA and cocoamidopropyl betaine.

A shampoo or other topical treatment in accordance with certain embodiments may include a thickening or suspending agent. Examples of thickening or suspending agents that may be contained in accordance with certain embodiments include carbomer and PEG 150 distearate. The thickening agent may be included to stabilize the shampoo during storage and/or to prevent the setting or dumping of pigments and silicone.

A pH adjuster or buffer may be included in a shampoo or other topical or oral treatment in accordance with certain embodiments. An example of a pH adjuster or buffer includes citric acid, tartaric and sodium hydroxide. The pH adjuster or buffer is configured to cause the shampoo to be gentle to the skin. A lower pH may cause hair to be compact and to shine and to protect the surfactant from hydrolysis, and as such, the pH adjuster or buffer may serve to lower the pH of the shampoo. However, alternative embodiments include pH adjusters that serve to raise the pH of a shampoo that contains an herbal formula that exhibits an exceptionally low pH.

An aesthetic additive may be included in a shampoo or other topical treatment in accordance with certain embodiments. Examples of aesthetic additives include colorants, opacifiers, UV absorbers, perfumes and natural and artificial fragrances.

One or more conditioners may be included in a shampoo in accordance with certain embodiments. The one or more conditioners may include a cationic polymer such as guar hydroxypropyl trimonium chloride. The one or more conditions may include silicone and/or a silicone emulsion such as dimethiconol, dimethicone, or amodimethicone. The silicone and/or silicone emulsion may serve to coat the hair and cause the hair to become soft, smooth and shiny.

A shampoo or other topical treatment in accordance with certain embodiments may include one or more active herbs or herbal extracts or emotives that are described in several examples herein. These one or more active herbs or herbal extracts serve to promote treatment of eczema, and/or another condition such as psoriasis, dermatitis, melanoma, hair loss and other hair or scalp conditions described herein or understood by those skilled in the art.

A preservative may be included in a shampoo or other topical or oral treatment in accordance with certain embodiments. The preservative may be configured to prevent microbial growth. Examples of preservatives that may be contained in a shampoo in accordance with certain embodiments include paraben free, formaldehyde donor free and halogenated free.

A moisturizer and/or one or more vitamins may be included in a shampoo or other topical treament in accordance with certain embodiments. Examples include combinations of D-Panthenol, vitamin E acetate, sodium PCA, glycerine and one or more amino acids. The moisturizer and/or vitamins may be configured to penetrate into hair shaft, seal cuticles and keep hair moisturized.

A shampoo or other topical treatment in accordance with certain embodiments may include a hydro-alcoholic hair, scalp or skin serum. Referring to FIG. 8, a hair growth cycle includes exogen, anagen, catagen and tetogen phases. The anagen phase involves active hair growth, whereby hair follicles regenerate and generate pigmented hair shafts. The telogen phase is a resting phase. The catagen phase involves cessation of hair growth and pigmentation, and release of papilla from the bulb. A hydro-alcoholic hair serum may be configured as a concentrated product that is typically left on the hair for a more extended duration than an ordinary shampoo with a typical shower routine. The hydro-alcoholic hair serum may be configured to be light and non-sticky on the scalp and as a non-irritant, to be light and non-sticky on the hair, to have little or no effect on hair volume, to strengthen scalp and DPC, to provide keratinization and collagen synthesis, to promote hair growth or to control hair fall, or combinations thereof. For example, the attributes of a hydro-alcoholic hair serum in accordance with certain embodiments may assist or promote treatment of eczema. A hydro-alcoholic hair serum in accordance with certain embodiments may include water, alcohol, humectant, solubilizer, water-based polymer, scalp conditioner, niacinamide, caffeine and panthenol. The ratio of alcohol, water and solubilizer may be adjusted depending of the solubilization power of the active herbal treatment composition.

A method is provided for treating eczema, and/or another skin, scalp or hair disorder such as psoriasis, dermatitis, melanoma, and/or hair loss. The treatment includes administering to a patient suffering from and/or diagnosed with eczema, psoriasis or other skin, scalp or hair disorder. The treatment includes combinations of certain herbs, herbal extracts, and/or compounds or molecules extracted from certain herbs or herbal extracts. Among these are herbs are one or more of Da Huang, Sheng Di Huang, and Jin Yin Hua, and/or one or more of Mu Dan Pi, Di Gu Pi, Xian He Cao, and Chun Gen Pi. Particularly studied among combinations of these herbs and others described herein include the three herbs Sheng Di Huang, Da Huang and Jin Yin Hua, three combinations of two of these three herbs, and formulae including one of these three herbs.

In one example, a shampoo or other topical or oral, IV or sub-dermal formulation may include between 2.5 wt. %-5.0 wt. % of a combination of Sheng Di Huang, Da Huang and Jin Yin Hua.

In another example, a shampoo or other topical or oral, IV or sub-dermal formulation may include between 1.0 wt. %-10.0 wt. % of a combination of Sheng Di Huang, Da Huang and Jin Yin Hua.

In another example, a shampoo or other topical or oral, IV or sub-dermal formulation may include between 1.0 wt. %-15.0 wt. % of a combination of Sheng Di Huang, Da Huang and Jin Yin Hua.

A treatment regimen may include combinations of applications of shampoo, cream and/or lotion to affected scalp and/or other skin areas and/or to hair.

Administration in a treatment regimen of certain combinations with one or two or more of these herbs serve to treat hair and scalp conditions as provided in accordance with embodiments described herein. Specific embodiments include advantageous combinations of Da Huang and Sheng Di Huang, as indicated below and in U.S. patent application Ser. No. 13/018,435, which is incorporated by reference, as well as with combinations including Jin Yin Hua with Da Huang and/or Sheng Di Huang. Further embodiments include combinations of DaHuang, Sheng Di Huang and/or Jin Yin Hua with one or more of Mu Dan Pi, Di Gu Pi, Xian He Cao, and/or Chun Gen Pi.

Further embodiments include combinations of beta-sitosterol or saw palmetto, or both, with Da Huang, Sheng Di Huang and/or Jin Yin Hua, and one or more of Mu Dan Pi, Di Gu Pi, Xian He Cao, and/or Chun Gen Pi and/or one or more other herbs or molecules described herein. Further embodiments include herbal combinations of one or more of Sheng Di Huang, Da Huang and Jin Yin Hua with combinations of one or more of emodin, digoxin, beta-sitosterol, saw palmetto, aucubin, rhein, rhapontin, vanillic acid, carvacrol or other herbs or molecules described herein or as understood by those skilled in the art. Other embodiments include combinations including one or more of more of Mu Dan Pi, Di Gu Pi, Xian He Cao, and/or Chun Gen Pi.

Treatment methods and medicinal compositions are provided for treating eczema, and other skin conditions. Certain embodiments involve the use of herbal combinations and combinations of certain herbs, certain herbal extracts and/or certain molecular components of certain herbs, alone or in combination with or supplemental to one or more other known or novel or experimental treatments. Certain embodiments are formulated as a shampoo, conditioner, cream, ointment or other topical scalp or hair treatment.

Methods and medicinal compositions are provided to treat eczema, and/or other skin conditions such as psoriasis, psoriatic arthritis, or other inflammatory skin disorders, dandruff including seborreic dandruff, seborrhea, acne, burns, dermatitis including atopic dermatitis, warts, keratosis, acne, alopecia, hirsutism or capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or warts or burns, or another skin ailment, lesion or sore, particularly of the scalp and also for other skin regions affected by an ailment that is susceptible to topical or hair treatment, as well as hair fall or hair loss conditions.

In certain embodiments, herbs are advantageously combined as herbal combinations including Da Huang and Sheng Di Huang. Certain embodiments also include Jin Yin Hua. Other embodiments include one or more of Mu Dan Pi, Di Gu Pi, Xian He Cao, and Chun Gen Pi. Certain embodiments include herbal extracts or molecular components such as emodin, digoxin, and/or other molecules such as aucubin, beta-sitosterol, vanillic acid, rhein, rhapontin and carvacrol. Combinations of these and other herbs, herbal extracts and/or molecules described herein are provided in various embodiments.

Along with certain combinations of herbs and/or herbal extracts, emotives or molecular components as described herein, a shampoo, conditioner, cream, ointment or other topical eczema treatment in accordance with certain embodiments may include one or more other components including one or more surfactants and/or co-surfactants, thickening agents, pH adjusters, buffers, aesthetic additives, water, hydro-alcoholic hair serum and/or another dispersive agent, solvent, solubilizer or vehicle, hair-fall or hair-loss control actives, conditioners, preservatives and/or moisturizers and/or vitamins, humectants, one or more cationic polymers, silicone, a suspending agent, perfume and/or other additives that may be consistent with a healthy shampoo, conditioner, cream, ointment or other topical eczema treatment.

A sulphate free shampoo may be formulated with the following ingredients:

An active herbal combination of Da Huang, Sheng Di Huang and Jin Yin Hua, aqua (DM water), PEG 80 Sorbitan Laurate, Sodium Lauroyl Sarcosinate, Disodium EDTA, Niacinamide, Citric Acid, Sodium Benzoate, and Phenoxy Ethanol.

In alternative embodiments, the active herbal combination may include Da Huang and Sheng Di Huang without Jin Yin Hua, or Da Huang and Jin Yin Hua without Sheng Di Huang, or Sheng Di Huang and Jin Yin Hua without Da Huang, or Da Huang alone with Sheng Di Huang nor Jin Yin Hua, or Sheng Di Huang alone without Da Huang nor Jin Yin Hua, or Jin Yin Hua alone without Da Huang nor Sheng Di Huang.

In alternative embodiments, a sulphate free shampoo may include PEG-4 Dilaurate, PEG-7 Glyceryl Cocoate, one or more PEG-20 Almond Glycerides, and/or PEG-25 Hydrogenated Castor Oil instead of or in addition to PEG 80 Sorbitan Laurate.

In alternative embodiments, a shampoo may include sodium lauryl sulphate instead of or in addition to Sodium Lauroyl Sarcosinate, although an eczema shampoo in accordance with these alternative embodiments would not be sulphate free.

In alternative embodiments, a sulphate free shampoo may include ascorbic acid instead of or in addition to citric acid.

The sulphate free shampoo may further include one or more of the following additional components: CAPB; or Glycerine, Diethylene Glycol, Propylene Glycol, and/or one or more Ceramides, Oils and/or Butters; or PEG 4 rapseedamide, Macrogol 200, and/or Polyoxyethylen(4); or Perfume allergen free and/or any GRAS natural perfume; or Poly Quat 10, Poly Quat 4, and/or Poly Quat 44; or PEG 150 Distearate, Antil 120 Plus, and/or Antil 127; or Olive Leaf Extract; Or combinations of two or more of these additional components.

A sulphate free shampoo may be formulated with the following ingredients:

An active herbal combination of Da Huang, Sheng Di Huang and Jin Yin Hua (6 mg), aqua (DM water), CAPB, PEG 80 Sorbitan Laurate, Sodium Lauroyl Sarcosinate, Glycerine, PEG 4 rapseedamide, Perfume allergen free, Disodium EDTA, Niacinamide, Poly Quat 10, Citric Acid, PEG 150 Distearate, Sodium Benzoate, Phenoxy Ethanol, and Olive Leaf Extract.

In alternative embodiments, the active herbal combination may include Da Huang and Sheng Di Huang without Jin Yin Hua, or Da Huang and Jin Yin Hua without Sheng Di Huang, or Sheng Di Huang and Jin Yin Hua without Da Huang, or Da Huang alone with Sheng Di Huang nor Jin Yin Hua, or Sheng Di Huang alone without Da Huang nor Jin Yin Hua, or Jin Yin Hua alone without Da Huang nor Sheng Di Huang.

An eczema shampoo may be formulated with the following ingredients:

An active herbal combination of Da Huang, Sheng Di Huang and Jin Yin Hua (6 mg), Aqua (D M Water), Sodium Laureth Sulphate 70%, Cocomonoethanolamide, Dimethiconol (and) Triethanolamine-Dodecylbenzenesulfonate, Ethylene Glycol Distearate, Sodium Chloride, D-Panthenol, Polyacrylic Acid 940, Guar Hydroxypropyltrimonium Chloride, Methylchloroisothiazolinone and Methylisothiazolinone, Sodium Hydroxide, Ethylenediaminetetraacetic acid tetrasodium salt, Tocopheryl acetate and Citric Acid.

In alternative embodiments, an eczema shampoo may include sodium lauryl sulphate instead of or in addition to sodium laureth sulphate 70%.

In alternative embodiments, an eczema shampoo may include SLES, SLS, ALES, and/or ALS instead of or in addition to Cocomonoethanolamide.

In alternative embodiments, an eczema shampoo may include glycol stearate and/or glycol stearate SE instead of or in addition to ethylene glycol distearate.

In alternative embodiments, an eczema shampoo may include Coconut oil and/or avocado oil instead of or in addition to D-Panthenol.

In alternative embodiments, an eczema shampoo may include Polyvinylalcohol (PVA) instead of or in addition to Polyacrylic Acid 940.

In alternative embodiments, an eczema shampoo may include Potassium hydroxide instead of or in addition to Sodium Hydroxide.

In alternative embodiments, an eczema shampoo may include ascorbic acid instead of or in addition to citric acid.

An eczema shampoo may also include perfume, perfume baby splash and/or any GRAS natural perfume.

An eczema shampoo may also include Cocoamidopropylbetaine instead of or in addition to Cocomonoethanolamide.

A hair lotion for treating eczema may be formulated as follows:

DM Water, an active herbal combination of Da Huang, Sheng Di Huang and Jin Yin Hua (6 mg), Propylene Glycol, Glyceryl Monostearate SE, Light Liquid Paraffin, Cetyl alcohol, Stearic acid, Cyclopentasiloxane, Phenoxyethanol &Triethylene glycol, Triethanolamine, Ethylenediaminetetraacetic acid tetrasodium salt and Alkyl Acrylates.

In alternative embodiments, an eczema shampoo may include 1,3-Propanediol instead of or in addition to Propylene Glycol.

In alternative embodiments, an eczema shampoo may include white soft paraffin instead of or in addition to Light Liquid Paraffin.

In alternative embodiments, an eczema shampoo may include cationic guar, one or more silicones, Honeyquat, and/or one or more polyquats instead of or in addition to Cetyl alcohol.

In alternative embodiments, an eczema shampoo may include Emulsifying wax, Beeswax instead of or in addition to stearic acid.

In alternative embodiments, an eczema shampoo may include C13-16 isoparaffin (and) C12-C14 isoparaffin (and) C13-C15 alkane instead of or in addition to Cyclopentasiloxane.

In alternative embodiments, an eczema shampoo may include Biotin, methyparaben, carbomer, cetyl alcohol, and/or propylparaben instead of or in addition to Triethanolamine.

A hair lotion for treating eczema may further include Coconut oil, olive oil, butter, bacon grease, peanut oil and/or or macadamia oil.

A hair lotion for treating eczema may also include perfume, perfume baby splash and/or any GRAS natural perfume.

A method of treating a scalp condition in accordance with certain embodiments includes applying to the scalp a medicinal composition formulated as a shampoo, conditioner, cream, ointment or other topical scalp or hair treatment that comprises a low concentration formula 5-10 grams or more of an active herbal combination of Sheng Di Huang, Da Huang and Jin Yin Hua in an example 16 fluid ounce formulation. A high concentration formula may include 50-150 grams of the active herbal combination in an example 16 fluid ounce formulation. A medium concentration formula may include 10-50 grams of the active herbal combination in an example 16 fluid ounce formulation.

In certain embodiments, the medicinal composition may include 1%-20% of the active herbal combination. In certain embodiments, the medicinal composition may include 2.5%-10% of the active herbal combination. In certain embodiments, the medicinal composition may include 2.5%-5.0% of the active herbal combination. In certain embodiments, the medicinal composition may include 3.5 wt. % or more of Da Huang. In certain embodiments, the medicinal composition may include 15.4 wt. % or more of a combination of Da Huang and Sheng Di Huang.

In certain embodiments, the herbal combination may include 2-20 grams of Jin Yin Hua, 2-20 grams of Da Huang and 6-60 grams of Sheng Di Huang. In certain embodiments, the medicinal composition may include 0.2 wt. %-4 wt. % of Jin Yin Hua, 0.2 wt. %-4 wt. % of Da Huang, and 0.6 wt. %-12 wt. % of Sheng Di Huang.

In certain embodiments, the herbal combination may include13.3-60 grams of Sheng Di Huang, 3.3-15 grams of Da Huang, and 3.3-15 grams of Jin Yin Hua.

In certain embodiments, the combination of Sheng Di Huang, Da Huang and Jin Yin Hua comprises 20 wt. %-80 wt. % of the active herbal combination. For example, the active herbal combination may include 12 wt. %-48 wt. % of Sheng di Huang, 4 wt. %-16 wt. % of Da Huang and 4 wt. %-16 wt. % of Jin Yin Hua.

The active herbal combination may include 18 wt. % or more of Sheng Di Huang, 6 wt. % or more of Da Huang and 6 wt. % or more of Jin Yin Hua.

A medicinal composition is also provided, in accordance with certain embodiments, that is formulated as a shampoo, conditioner, cream, ointment or other topical scalp or hair treatment, comprising an herbal combination of 13.3 grams or more of Sheng Di Huang and 3.3 grams or more of Da Huang in a 16 fluid ounce formulation. In certain embodiment, the herbal combination also includes 3.3 grams of Jin Yin Hua.

The medicinal composition may include 0.5 wt. % or more of Da Huang and 1.5 wt. % or more of Sheng Di Huang. In certain embodiments, the medicinal composition also includes 0.5 wt. % or more of Jin Yin Hua.

The medicinal composition may include 0.5 wt. %-1.5 wt. % of Da Huang and 1.5 wt. %-4.5 wt. % of Sheng Di Huang. In certain embodiments, the medicinal composition also includes 0.5 wt. %-1.5 wt. % of Jin Yin Hua.

The medicinal composition may include 6 wt. % or less of Da Huang and 18 wt. % or less of Sheng Di Huang. In certain embodiments, the medicinal composition includes 6 wt. % or less Jin Yin Hua.

The active herbal combination may include 40 wt. % or more of Sheng Di Huang and 10 wt. % or more of Da Huang. In certain embodiments, the active herbal combination may include 10 wt. % or more Jin Yin Hua.

The active herbal combination within the medicinal composition may include 22.4 wt. % or more, 30 wt. % or more or 60 wt. % or more of the combination of Sheng Di Huang, Da Huang and Jin Yin Hua.

The active herbal combination may include 15.4 wt. % or more of Sheng Di Huang, 3.5 wt. % or more of Da Huang and 3.5 wt. % or more of Jin Yin Hua.

The active herbal combination may include 18 wt. % or more of Sheng Di Huang, 6 wt. % or more of Da Huang and 6 wt. % or more of Jin Yin Hua.

Other medicinally active or inactive components may be included in combination with the herbal combinations described herein. The herbal combination may be administered alone or in combination with or supplemental to one or more other medicinal treatments.

Referring to FIG. 1, an example of a shampoo in accordance with certain embodiments is illustrated as including nine generalized components. These nine components include a surfactant, a thickening agent, a pH adjuster/buffer, an aesthetic additive, water, a conditioner, one or more active herbs or herbal extracts or molecules, a preservative and moisturizers/vitamins. Shampoos in accordance with various alternative formulations may include fewer than all of these nine components and they may include other active or inactive components known to those skilled in the art as having some advantage when included in a shampoo formula or in a medicinal combination for treating eczema, or another skin condition such as psoriasis, melanoma, or dermatitis or hair loss or another head or scalp disorder or ailment.

A shampoo or other skin or scalp or hair treatment in accordance with certain embodiments may include one or more surfactants that may be known or discovered as being advantageous for cleaning hair with a shampoo. A primary surfactant may be included to provide flash foam for cleaning the hair by removing dirt and other impurities. A secondary surfactant may be included to provide stable foam and to reduce the harshness of the primary surfactant. A surfactant may be used that includes a charged, hydrophilic head group and a long, hydrophobic alkyl chain tail. Surfactants are configured to break molecular bonds between dirt and hair and to transport the dirt into an aqueous medium to be rinsed free from the hair and scalp. Examples of surfactants that may be contained in a shampoo in accordance with certain embodiments include sodium laureth sulphate, ammonium laureth sulfate, and sodium cocoyl isethionate. Examples of co-surfactants include cocamide MEA and cocoamidopropyl betaine.

A shampoo in accordance with certain embodiments may include a thickening or suspending agent. Examples of thickening or suspending agents that may be contained in accordance with certain embodiments include carbomer and PEG 150 distearate. The thickening agent may be included to stabilize the shampoo during storage and/or to prevent the setting or dumping of pigments and silicone.

A pH adjuster or buffer may be included in a shampoo in accordance with certain embodiments. An example of a pH adjuster or buffer includes citric acid, tartaric and sodium hydroxide. The pH adjuster or buffer is configured to cause the shampoo to be gentle to the skin. A lower pH may cause hair to be compact and to shine and to protect the surfactant from hydrolysis, and as such, the pH adjuster or buffer may serve to lower the pH of the shampoo. However, alternative embodiments include pH adjusters that serve to raise the pH of a shampoo that contains an herbal formula that exhibits an exceptionally low pH.

An aesthetic additive may be included in a shampoo in accordance with certain embodiments. Examples of aesthetic additives include colorants, opacifiers, UV absorbers, perfumes and natural and artificial fragrances.

One or more conditioners may be included in a shampoo in accordance with certain embodiments. The one or more conditioners may include a cationic polymer such as guar hydroxypropyl trimonium chloride. The one or more conditions may include silicone and/or a silicone emulsion such as dimethiconol, dimethicone, or amodimethicone. The silicone and/or silicone emulsion may serve to coat the hair and cause the hair to become soft, smooth and shiny.

A shampoo in accordance with certain embodiments includes one or more active herbs or herbal extracts or emotives that are described in several examples herein. These one or more active herbs or herbal extracts serve to promote treatment of eczema, and/or another condition such as psoriasis, dermatitis, melanoma, hair loss and other hair or scalp conditions described herein or understood by those skilled in the art.

A preservative may be included in a shampoo in accordance with certain embodiments. The preservative may be configured to prevent microbial growth. Examples of preservatives that may be contained in a shampoo in accordance with certain embodiments include paraben free, formaldehyde donor free and halogenated free.

A moisturizer and/or one or more vitamins may be included in a shampoo in accordance with certain embodiments. Examples include combinations of D-Panthenol, vitamin E acetate, sodium PCA, glycerine and one or more amino acids. The moisturizer and/or vitamins may be configured to penetrate into hair shaft, seal cuticles and keep hair moisturized.

A shampoo in accordance with certain embodiments may include a hydro-alcoholic hair serum. Referring to FIG. 8, a hair growth cycle includes exogen, anagen, catagen and tetogen phases. The anagen phase involves active hair growth, whereby hair follicles regenerate and generate pigmented hair shafts. The telogen phase is a resting phase. The catagen phase involves cessation of hair growth and pigmentation, and release of papilla from the bulb. A hydro-alcoholic hair serum may be configured as a concentrated product that is typically left on the hair for a more extended duration than an ordinary shampoo with a typical shower routine. The hydro-alcoholic hair serum may be configured to be light and non-sticky on the scalp and as a non-irritant, to be light and non-sticky on the hair, to have little or no effect on hair volume, to strengthen scalp and DPC, to provide keratinization and collagen synthesis, to promote hair growth or to control hair fall, or combinations thereof. For example, the attributes of a hydro-alcoholic hair serum in accordance with certain embodiments may assist or promote treatment of eczema. A hydro-alcoholic hair serum in accordance with certain embodiments may include water, alcohol, humectant, solubilizer, water-based polymer, scalp conditioner, niacinamide, caffeine and panthenol. The ratio of alcohol, water and solubilizer may be adjusted depending of the solubilization power of the active herbal treatment composition.

A method is provided for treating eczema, and/or another skin, scalp or hair disorder such as psoriasis, dermatitis, melanoma, and/or hair loss. The treatment includes administering to a patient suffering from and/or diagnosed with eczema, psoriasis or another skin, scalp or hair disorder. The treatment includes combinations of certain herbs, herbal extracts, and/or compounds or molecules extracted from certain herbs or herbal extracts. Among these are herbs are one or more of Da Huang, Sheng Di Huang, and Jin Yin Hua, and/or one or more of Mu Dan Pi, Di Gu Pi, Xian He Cao, and Chun Gen Pi. Particularly studied among combinations of these herbs and others described herein include the three herbs Sheng Di Huang, Da Huang and Jin Yin Hua, three combinations of two of these three herbs, and formulae including one of these three herbs.

In one example, a shampoo formulation may include between 2.5 wt. %-5.0 wt. % of a combination of Sheng Di Huang, Da Huang and Jin Yin Hua.

In another example, a shampoo formulation may include between 1.0 wt. %-10.0 wt. % of a combination of Sheng Di Huang, Da Huang and Jin Yin Hua.

In another example, a shampoo formulation may include between 1.0 wt. %-15.0 wt. % of a combination of Sheng Di Huang, Da Huang and Jin Yin Hua.

A treatment regimen may include combinations of applications of shampoo, cream and/or lotion to affected scalp or other skin areas.

### INCORPORATION BY REFERENCE

What follows is a cite list of references which are, in addition to those references cited above and below herein, and including that which is described as background, the invention summary, brief description of the drawings, the drawings and the abstract, hereby incorporated by reference into the detailed description of the preferred embodiments below, as disclosing alternative embodiments of elements or features of the preferred embodiments not otherwise set forth in detail below. A single one or a combination of two or more of these references may be consulted to obtain a variation of the preferred embodiments described in the detailed description below. Further patent, patent application and non-patent references are cited in the written description and are also incorporated by reference into the preferred embodiments.

A treatment regimen for psoriasis, eczema, inflammation, autoimmune disease, melanoma or other skin ailment, leukemia or other cancer, or other disease including methotrexate, betamethasone or another known treatment described herein, together with administering, before, during and/or after medicinal doses of such known treatment, combinations of the herbs and/or molecules described herein may also be combined with other treatments such as may be understood by those skilled in the art and/or as may be described in literature such as the following which are hereby incorporated by reference, along with the background and brief descriptions of the drawings and priority and related applications, as disclosing alternative embodiments and compounds that may be combined with an herbal and/or molecular combination and a known or discovered treatment or other described treatment in a cocktail or other combinative therapy:

Chronic Lymphocytic Leukemia by the Leukemia & Lymphoma Society;

Medifocus.com Medifocus Guide on Chronic Lymphocytic Leukemia;

Ranjit Thomas, et al., Spontaneous Clinical Regression in Chronic Lymphocytic Leukemia, British Journal of Haematology, 2002, 116, 341-345;

Dragomir Marisavljevic, et al., Spontaneous Clinical Remission of Chronic Lymphocytic Leukemia, Haema 2003; 6(3): 394-397;

Upshaw J D Jr, et al., Spontaneous Remission of B cell Chronic Lymphocytic Leukemia associated with T Lymphocytic Hyperplasia in bone marrow, South Med J. 2002 June 1995(6): 647-9;

Wiernik P H, Second neoplasms in patients with chronic lymphocytic leukemia, Current Treat Options Oncol. June 2004; 5(3):215-23;

Luis Fayad M D and Susan O'Brien M D, Chronic Lymphocytic Leukemia and Associated Disorders, Medical Oncology: A Comprehensive Review, 1995;

Michael J. Keating, et al., Biology and Treatment of Chronic Lymphocytic Leukemia, American Society of Hematology, Hematology 2003, 153-175;

G E Marti, et al, Descriptive Epidemiology of Chronic Lymphocytic Leukemia (CLL);

Sarfaraz K. Niazi, Handbook of Pharmaceutical Manufacturing Formulations, Culinary & Hospitality Industry Publications Services; and

U.S. Pat. Nos. 5,872,103; 6,197,754; 6,740,665; 6,812,255; 7,268,162; 7,358,222; 7,381,535; 7,393,656; 7,563,584; 7,695,926; 7,790,905; 8,541,382; 8,547,695; 8,734,859; and

United States published patent applications serial nos. 20030211180; 20050008664; 20050026849; 20050196473; 20060205679; 20070191262; 20080152700; 20080220441; 20090018088; 20090143279; 20090215042; 20090269772; 20100068198; 20100092585; 20100144647; 20100167286; 20120122807; and

PCT published applications no. WO01/66123A2; WO2004/052294A2; WO2006/053049A2; WO2007/130124A1; WO2012/063134A2.

Administration in a treatment regimen of certain combinations with one or two or more of these herbs serve to treat hair and scalp conditions as provided in accordance with embodiments described herein. Specific embodiments include advantageous combinations of Da Huang and Sheng Di Huang, as indicated below and in any one or a combination of U.S. patent applications Nos. 61/413,430; 62/325,993; 62/313,709; 62/268,226; 62/259,056; 62/348,762; 15/133,056; 15/131,743; 62/297,796; 62/198,637; 14/754,266; 14/710,865; 14/815,892; 14/287,158; 14/287,153; 13/890,990; 14/981,899; 14/815,705; 13/152,039; PCT/IB11/03078; PCT/IB13/02975; PCT/US15/38341; and US published patent applications nos. 20160051553; 20160136220; 20160136219; 20160136216; 20160136223; 20160136222; 20160136221; 20160113983; 20160143980; 20160113982; 20160136218; 20140205685; and 20140206631; and U.S. Pat. Nos. 9,066,974; 9,095,606; 8,734,859; 8,597,695; and 8,541,382; which are each incorporated by reference, as well as with combinations including Jin Yin Hua with Da Huang and/or Sheng Di Huang. Further embodiments include combinations of Da Huang, Sheng Di Huang and/or Jin Yin Hua with one or more of Mu Dan Pi, Di Gu Pi, Xian He Cao, and/or Chun Gen Pi.

Further embodiments include combinations of beta-sitosterol or saw palmetto, or both, with Da Huang, Sheng Di Huang and/or Jin Yin Hua, and one or more of Mu Dan Pi, Di Gu Pi, Xian He Cao, and/or Chun Gen Pi and/or one or more other herbs or molecules described herein. Further embodiments include herbal combinations of one or more of Sheng Di Huang, Da Huang and Jin Yin Hua with combinations of one or more of emodin, digoxin, beta-sitosterol, saw palmetto, aucubin, rhein, rhapontin, vanillic acid, carvacrol or other herbs or molecules described herein or as understood by those skilled in the art. Other embodiments include combinations including one or more of more of Mu Dan Pi, Di Gu Pi, Xian He Cao, and/or Chun Gen Pi.

Contained within each of the seven herbs are several molecular constituents. Observed reductions of psoriatic inflammation and other studied effects owing to a treatment regimen of periodic shampooing with an herbal formula in accordance with the embodiments can be as a result of various combinations of active molecules contained in Da Huang, Jin Yin Hua and/or Sheng Di Huang, and of combinations of the herbs themselves.

Contained within each of the seven herbs are several molecular constituents. Observed reductions of psoriatic inflammation and other studied effects owing to a treatment regimen of periodic shampooing with an herbal formula in accordance with the embodiments can be as a result of various combinations of active molecules contained in Da Huang, Jin Yin Hua and/or Sheng Di Huang, and of combinations of the herbs themselves.

It is contemplated, as people with ordinary skill in the art would do, that the newly separated compounds may be each individually or in combination used as an ingredient to prepare a pharmaceutical composition for a particular treatment purpose. As it is the status of the art in the pharmaceutical industry, once substantially pure preparations of a compound are obtained, various pharmaceutical compositions or formulations can be prepared from the substantially pure compound using conventional processes or future developed processes in the industry. Specific processes of making pharmaceutical formulations and dosage forms (including, but not limited to, tablet, capsule, injection, syrup) from chemical compounds are not part of the invention and people of ordinary skill in the art of the pharmaceutical industry are capable of applying one or more processes established in the industry to the practice of the present invention. Alternatively, people of ordinary skill in the art may modify the existing conventional processes to better suit the compounds of the present invention. For example, the patent or patent application databases provided at USPTO official website contain rich resources concerning making pharmaceutical formulations and products from effective chemical compounds. Another useful source of information is Handbook of Pharmaceutical Manufacturing Formulations, edited by Sarfaraz K. Niazi and sold by Culinary & Hospitality Industry Publications Services, which is incorporated by reference.

While the invention has been described in terms of several embodiments, those skilled in the art will recognize that the invention is not limited to the embodiments described, but can be practiced with modification and alteration within the spirit and scope of the appended claims. The description is thus to be regarded as illustrative instead of limiting of the invention as set forth in the appended claims including structural and functional equivalents thereof.

We claim:

1. A method of treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, comprising:
measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a first bodily fluid, serum or skin sample, or both, extracted from a patient;
formulating a diagnosis of scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, for the patient based on said measured level or combination of levels and on one or more expected correlations between said level or combination of levels and manifestation within said patient of one or more diseases; and
administering a medicinal composition that comprises an effective dose between 20-160 mg/kg of an herbal combination of Da Huang, Sheng Di Huang and Jin Yin Hua to the patient to treat the patient in accordance with said diagnosis.

2. The method of claim 1, further comprising:
measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid, serum or skin sample, or both, extracted from the patient after administering said medicinal composition over a prognostic period; and
indicating to repeat the administering of said medicinal composition that comprises an effective dose between 20-160 mg/kg of an herbal combination of Da Huang, Sheng Di Huang and Jin Yin Hua to said patient a significant number of further times based on a comparison between said measured levels or combinations of levels within said first and second bodily fluid, serum or skin samples, or both, and on an expected correlation between certain differences between measured levels or combinations of levels in bodily fluid samples respectively extracted before and after a prognostic period of administration of said medicinal composition.

3. The method of claim 1, wherein the medicinal composition comprises between 1.0 wt. %-15 wt. % of said herbal combination of Da Huang, Sheng Di Huang and Jin Yin Hua.

4. The method of claim 3, wherein the medicinal composition comprises 2.5 wt. % or more of said herbal combination of Da Huang, Sheng Di Huang and Jin Yin Hua.

5. The method of claim 4, wherein the medicinal composition comprises 10 wt. % or less of said herbal combination of Da Huang, Sheng di Huang and Jin Yin Hua.

6. The method of claim 1, wherein the medicinal composition comprises 2.5 wt. %-5.0 wt. % of said herbal combination of Da Huang, Sheng Di Huang and Jin Yin Hua.

7. The method of claim 1, wherein the medicinal composition comprises an effective dose between 1-6 grams of said herbal combination for a 50 kg patient or 2-12 grams for a 100 kg patient.

8. The method of claim 7, wherein said effective dose comprises 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

9. The method of claim 1, wherein the medicinal composition is formulated as a shampoo and applied to the scalp.

10. The method of claim 1, wherein the medicinal composition is formulated as a shampoo, conditioner, cream, lotion, ointment or other topical scalp or hair treatment.

11. The method of claim 1, wherein the medicinal composition is formulated as a cream, lotion, ointment or other topical skin treatment.

12. The method of claim 1, further comprising administering, in combination with said medicinal composition that comprises effective doses between 20-160 mg/kg of an herbal combination of Da Huang, Sheng Di Huang and Jin Yin Hua, one or more additional medicines including Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof.

13. The method of claim 1, further comprising administering, in combination with said medicinal composition that comprises effective doses between 20-160 mg/kg of an herbal combination of Da Huang, Sheng Di Huang and Jin Yin Hua, one or more additional medicines including an IL-12, IL-23, TNF alpha, JAK, STAT, IL-17, PDE4, p40 subunit of IL-12 and/or IL-23 and/or a humanized p40 monoclonal antibody, IL-22, IL-20, IL-23/p19, JAK3, Th1, Th17 and/or Th22 cell, IFN gamma, IL-17R, IL-19, sPLA2, NO (nitric oxide), VEGF, IL-24, kinase, tyrosine, topoisomerase, IL-1, IL-6, IL-8, BTK, SYK, ZAP-70, PI3KCD, AKT, HER-2, FLT-3, MAPK1, BRAF, MEK1, PD-1, CD279, PD-L1, LYN, ABL, FLT3, KIT, LCK, JAK1, PLC, BCR, P13K delta, GMCSF, MCP-1, AKT1 (PKB alpha), ERBB2 (HER2), FLT3, MAPK1 (ERK2), PRKCA (PKC alpha), BRAF, BRAF V599E or MAP2K1 (MEK1) inhibitor, or combinations thereof.

14. A method of treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (MS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, comprising:
measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, ORA KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a first bodily fluid, serum or skin sample, or both, extracted from a patient;
formulating a diagnosis of scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, for the patient based on said measured level or combination of levels and on one or more expected correlations between said level or combination of levels and manifestation within said patient of one or more diseases; and
administering a medicinal composition that comprises an effective dose between 1.0 wt. %-15 wt. % of an herbal combination of Da Huang, Sheng Di Huang and Jin Yin Hua for a 50 kg patient or 2-12 grams for a 100 kg patient to treat the patient in accordance with said diagnosis.

15. The method of claim 14, further comprising:
measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4,MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid, serum or skin sample, or both, extracted from the patient after administering said medicinal composition over a prognostic period; and
indicating to repeat the administering of effective doses of said medicinal composition to said patient a significant number of further times based on a comparison between said measured levels or combinations of levels within said first and second bodily fluid, serum or skin samples, or both, and on an expected correlation between certain differences between said measured levels or combinations of levels in bodily fluid samples respectively extracted before and after the prognostic period of administration of said medicinal composition.

16. The method of claim 14, wherein the medicinal composition comprises between 1-6 grams of said herbal combination of Da Huang, Sheng Di Huang and Jin Yin Hua for a 50 kg patient or 2-12 grams for a 100 kg patient.

17. The method of claim 14, wherein the medicinal composition comprises 2.5 wt. % or more of said herbal combination of Da Huang, Sheng Di Huang and Jin Yin Hua.

18. The method of claim 17, wherein the medicinal composition comprises 10 wt. % or less of said herbal combination of Da Huang, Sheng di Huang and Jin Yin Hua.

19. The method of claim 14, wherein the medicinal composition comprises 2.5 wt. %-5.0 wt. % of said herbal combination of Da Huang, Sheng Di Huang and Jin Yin Hua.

20. The method of claim 14, wherein said effective dose comprises 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

21. The method of claim 14, wherein the medicinal composition is formulated as a shampoo and applied to the scalp.

22. The method of claim 14, wherein the medicinal composition is formulated as a shampoo, conditioner, cream, lotion, ointment or other topical scalp or hair treatment.

23. The method of claim 14, wherein the medicinal composition is formulated as a cream, lotion, ointment or other topical skin treatment.

24. The method of claim 14, further comprising administering, in combination with said medicinal composition that comprises effective doses between 1.0 wt. %-15 wt. % of an herbal combination of Da Huang, Sheng Di Huang and Jin Yin Hua, one or more additional medicines including Etanercept, Betamethotrexate, Methotrexate, 5-fluoroviacil, Paclitaxel, Adriamycin (doxorubicin), Etoposide, 5-fluorourasil, Docetaxil, Vincristine, Irinotecan, Methylprednisolone, Carboplatin, Dacarbazine, Acitretin, Glycyrrhizin, Paeonia lactiflora, Glycyrrhiza uralensis, Mahonia aquafolium, Aloe vera, Indigo naturalis, Kaku nut oil, Camptotheca acuminatanut, Calcipotriol and tazarotene gel, Otezla, Embrel, Humira, Cinzia, Cosentyx, Remicade, Simponi, Taltz, one or more steroids, Tacrolimus, Prograf, or cyclosporine, or combinations thereof.

25. The method of claim 14, further comprising administering, in combination with said medicinal composition that comprises effective doses between 1.0 wt. %-15 wt. % of an herbal combination of Da Huang, Sheng Di Huang and Jin Yin Hua, one or more additional medicines including an IL-12, IL-23, TNF alpha, JAK, STAT, IL-17, PDE4, p40 subunit of IL-12 and/or IL-23 and/or a humanized p40 monoclonal antibody, IL-22, IL-20, IL-23/p19, JAK3, Th1, Th17 and/or Th22 cell, IFN gamma, IL-17R, IL-19, sPLA2, NO (nitric oxide), VEGF, IL-24, kinase, tyrosine, topoisomerase, IL-1, IL-6, IL-8, BTK, SYK, ZAP-70, PI3KCD, AKT, HER-2, FLT-3, MAPK1, BRAF, MEK1, PD-1, CD279, PD-L1, LYN, ABL, FLT3, KIT, LCK, JAK1, PLC, BCR, P13K delta, GMCSF, MCP-1, AKT1 (PKB alpha), ERBB2 (HER2), FLT3, MAPK1 (ERK2), PRKCA (PKC alpha), BRAF, BRAF V599E or MAP2K1 (MEK1) inhibitor, or combinations thereof.

26. A medicine for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (MS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, comprising:
a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose including between 20-160 mg/kg of an herbal combination of Da Huang, Sheng Di Huang and Jin Yin Hua.

27. The medicine of claim 26, wherein each effective dose comprises between 1-6 grams of said herbal combination for a 50 kg patient or 2-12 grams for a 100 kg patient.

28. The medicine of claim 27, wherein said each effective dose comprises 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

29. The medicine of claim 26, wherein said each effective dose comprises 1.0 wt. %-15.0 wt. % of said herbal combination of Da Huang, Sheng Di Huang and Jin Yin Hua.

30. The medicine of claim 26, wherein said each effective dose comprises 2.5 wt. %-5.0 wt. % of said herbal combination of Da Huang, Sheng Di Huang and Jin Yin Hua.

31. A medicine for treating scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof, comprising
a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose including between 1.0 wt. %-15.0 wt. % of said herbal combination of Da Huang, Sheng Di Huang and Jin Yin Hua.

32. The medicine of claim 31, wherein said each effective dose comprises 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

33. The medicine of claim 31, wherein said each effective dose comprises 1-6 grams of said herbal combination for a 50 kg patient or 2-12 grams for a 100 kg patient.

34. The medicine of claim 31, wherein said each effective dose comprises 2.5 wt. %-5.0 wt. % of said herbal combination of Da Huang, Sheng Di Huang and Jin Yin Hua.

35. A medical kit, comprising:
a diagnostic kit, including a test kit and an indicator, said test kit for measuring a level or combination of levels of one or more of IL-5, IL-13, IL-17, IL-23, TNF-α, IL-6, IL-7, IL-13, GMCSF, MCP-1, IL-9, IL-113, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4,MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a bodily fluid, serum or skin sample, or both, and said indicator for providing a diagnostic result based on said measured level or combinations of levels and on one or more expected correlations between said level or combination of levels and manifestation of one or more diseases including scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (IBS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof; and
a medicinal formulation comprising shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, said medicinal formulation comprising a predetermined number of one or more effective doses, each effective dose including between 20-160 mg/kg of an herbal combination of Da Huang, Sheng Di Huang and Jin Yin Hua.

36. The medical kit of claim 35, further comprising a prognostic kit including a prognostic test kit and a prognostic indicator, said prognostic test kit for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Me1-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, ORA KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid, serum or skin sample, or both, after a prognostic period of treatment, and said prognostic indicator for providing a prognostic result for the patient based on a comparison of said measured level or combination of levels in the first and second bodily fluid, serum or skin sample, or both.

37. A medical kit, comprising:
a diagnostic kit, including a test kit and an indicator, said test kit for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Me1-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a bodily fluid, serum or skin sample, or both, and said indicator for providing a diagnostic result based on said measured level or combinations of levels and on one or more expected correlations between said level or combination of levels and manifestation of one or more diseases including scalp psoriasis, dermatitis, atopic dermatitis, eczema, herpes, shingles, rheumatoid arthritis (RA), arthritic psoriasis/psoriatic arthritis, Alzheimer's, Parkinson's, irritable bowel syndrome (MS), colitis, prostitis, vasculitis, osteoarthritis, seborrheic dermatitis, acne, colitis, skin lesions, diabetes, hypertension, allergies, asthma, capuche sarcoma, autoimmune, inflammation, dermatologic, cardiovascular, metabolic syndrome, hypotension, coronary artery disease, depression, lupus, sarcoidosis, muscular sclerosis, crohn's disease, UV exposure, burns, warts, dandruff, seborrheic dandruff, chronic inflammation, seborrhea, keratosis, alopecia, hirsutism, capitis, melanoma or non-melanoma skin cancer, basal cell cancer (BCC), squamous cell cancer (SCC), scleroderma, dermatofibrosarcoma protuberans, Merkel cell carcinoma, Kaposi's sarcoma, keratoacanthoma, spindle cell tumors, sebaceous carcinomas, microcystic adnexal carcinoma, Paget's disease of the breast, atypical fibroxanthoma, leiomyosarcoma, and angiosarcoma, Marjolin's ulcers, kidney failure, nerve damage caused by a skin condition, or skin burrowing mites, or a skin ailment, lesion or sore, particularly of the scalp and also for other skin regions affected by an ailment that is susceptible to topical or hair treatment, as well as hair fall or hair loss conditions, or combinations thereof; and
a medicinal formulation comprising shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, said medicinal formulation comprising a predetermined number of one or more effective doses, each effective dose including between 1.0 wt. %-15 wt. % of an herbal combination of Da Huang, Sheng di Huang and Jin Yin Hua.

38. The medical kit of claim 37, further comprising a prognostic kit including a prognostic test kit and a prognostic indicator, said prognostic test kit for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, f32M, NO, LTB4, PLA2, or PDE4, ORA KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in a second bodily fluid, serum or skin sample, or both, after a prognostic period of treatment, and said prognostic indicator for providing a prognostic result for the patient based on a comparison of said measured level or combination of levels in the first and second bodily fluid, serum or skin sample, or both.

39. The medical kit of claim 37, wherein said each effective dose comprises 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

40. The medical kit of claim 37, wherein said each effective dose comprises 1-6 grams of said herbal combination for a 50 kg patient or 2-12 grams for a 100 kg patient.

41. The medical kit of claim 37, wherein said each effective dose comprises 2.5 wt. %-5.0 wt. % of said herbal combination of Da Huang, Sheng Di Huang and Jin Yin Hua.

42. The medical kit of claim 41, wherein said each effective dose comprises between 20-160 mg/kg of an herbal combination of Da Huang, Sheng Di Huang and Jin Yin Hua.

43. The medical kit of claim 37, wherein said each effective dose comprises between 20-160 mg/kg of an herbal combination of Da Huang, Sheng Di Huang and Jin Yin Hua.

44. A prognostic kit, comprising:
a medicinal formulation comprising shampoo, conditioner, cream, lotion, ointment or other topical skin, scalp or hair treatment or a pill, skin patch, gel, injection pen, subdermal injection packet, IV fluid package, tablet, capsule, lipid carrier, nano-crystal or other nano-particulate formulation, subcutaneous insert, or stent, or combinations thereof, said medicinal formulation comprising a predetermined number of one or more effective doses, each effective dose including between 20-160 mg/kg of an herbal combination of Da Huang, Sheng Di Huang and Jin Yin Hua, and
a prognostic test kit for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-65 , IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, OR A KINASE, E.G., AKT1 (PKB ALPHA), ERBB2 (HER2), FLT3, MAPK1 (ERK2), PRKCA (PKC ALPHA), BRAF, BRAF V599E OR MAP2K1 (MEK1), or combinations thereof, in first and second bodily fluid, serum or skin samples, or both, respectively extracted from a patient before and after a prognostic period of treatment with said medicinal formulation, and a prognostic indicator for providing a prognostic result for the patient based on a comparison of said measured level or combination of levels in the first and second bodily fluid, serum or skin samples, or both.

45. The prognostic kit of claim 44, wherein said each effective dose comprises 1.0 wt. %-15.0 wt. % of said herbal combination of Da Huang, Sheng Di Huang and Jin Yin Hua.

46. The prognostic kit of claim 44, wherein said each effective dose comprises 2.5 wt. %-5.0 wt. % of said herbal combination of Da Huang, Sheng Di Huang and Jin Yin Hua.

47. A prognostic kit, comprising:
a shampoo, conditioner, cream, lotion, ointment or other topical skin, scalp or hair treatment or a pill, skin patch, gel, injection pen, subdermal injection, IV fluid, tablet, capsule, lipid carrier, nano-crystal or other nano-particulate formulation, subcutaneous insert, or stent, or combinations thereof, that comprises a predetermined number of one or more effective doses, each effective dose including between 1.0 wt. %-15.0 wt. % of an herbal combination of Da Huang, Sheng Di Huang and Jin Yin Hua; and
a prognostic test kit for measuring a level or combination of levels of one or more of IL-5, IL-13, IL-17, IL-23, TNF-α, IL-6, IL-7, IL-13, GMCSF, MCP-1, IL-9, IL-1-β, IL-1-RA, IL-8, IL-9, IL-10, IL-12, IL-19, IL-20, IL-22, IL-24, IL-26, eotaxin, FGF-β, G-CSF, IFN-γ, IP-10, MIP-1, PDGFRB, MIP-1-β, RANTES, VEGF, JAK, JAK1, JAK3, MPO (myeloperoxidase), p38kinase, BRAF, BRAF V599E, KRAS, EGFR, ALK, TRPV1, TRPA1, MRGPRA3, PAR2, mucunain or other proteases, chloroquine, pruritogen, cowhage, sapsaicin, ROS, RET. SK-Mel-2, HT-29, MCF-7, THP-1, DU-145, MOLT-4, THP-1, K562, HL-60, U937, PD-1, ICAM-1, VCAM-1, E-selectin, HACAT-THP-1, LYN, ABL, BTK, SYK, ZAP-70, PI3KCD, AKT, HER-2, FLT-3, KIT, LCK, MAPK1, MEK1, PGE, PGE2, PGE2-E4, MMP, PI3K-δ, PIK3CD, PIK3R1 CCL3, CCL4, PLC, BCR, CD-5, CD-38, CD-19, CD-20, CD-79a, β2M, NO, LTB4, PLA2, or PDE4, or a kinase, e.g., AKT1 (PKB alpha), ERBB2 (HER2), FLT3, MAPK1 (ERK2), PRKCA (PKC alpha), BRAF, BRAF V599E or MAP2K1 (MEK1), or combinations thereof, in first and second bodily fluid, serum or skin samples, or both, respectively extracted from a patient before and after a prognostic period of treatment with said medicinal formulation, and a prognostic indicator for providing a prognostic result for the patient based on a comparison of measured levels or combinations of levels from said first and second bodily fluid, serum or skin samples, or both.

48. The prognostic kit of claim 47, wherein said effective doses each comprise 0.2-1.2 grams of Da Huang, 0.6-3.6 grams of Sheng Di Huang and 0.2-1.2 grams of Jin Yin Hua for a 50 kg patient or 0.4-2.4 grams of Da Huang, 1.2-7.2 grams of Sheng Di Huang and 0.4-2.4 grams of Jin Yin Hua for a 100 kg patient.

49. The prognostic kit of claim 47, wherein said each effective dose comprises 1-6 grams of said herbal combination for a 50 kg patient or 2-12 grams for a 100 kg patient.

50. The prognostic kit of claim 47, wherein said each effective dose comprises 2.5 wt. %-5.0 wt. % of said herbal combination of Da Huang, Sheng Di Huang and Jin Yin Hua.

51. The prognostic kit of claim 47, wherein said each effective dose comprises between 20-160 mg/kg of an herbal combination of Da Huang, Sheng Di Huang and Jin Yin Hua.
